(19) United States
(12) Patent Application Publication
(10) Pub. No.: US 2025/0382606 A1
(43) Pub. Date: Dec. 18, 2025

(54) METHODS AND SYSTEMS FOR IDENTIFYING NOVEL ALLOSTERIC TRANSCRIPTION FACTOR OPERATORS, AND NOVEL NUCLEIC ACIDS

(71) Applicant: Northwestern University, Evanston, IL (US)

(72) Inventors: Steven R. Fleming, Evanston, IL (US); Michael Christopher Jewett, Evanston, IL (US)

(21) Appl. No.: 18/878,240

(22) PCT Filed: Jun. 23, 2023
(86) PCT No.: PCT/US2023/068975

§ 371 Date: Dec. 23, 2024

Related U.S. Application Data
(60) Provisional application No. 63/354,837, filed on Jun. 23, 2022.

(51) Int. Cl. C12N 15/10

(52) U.S. Cl. CPC C12N 15/1065; C12N 15/1086

(57) ABSTRACT

Disclosed herein are methods and systems to generate novel nucleic acid sequences that bind to an allosteric transcription factor and novel nucleic acid sequences generated by said methods.

### CROSS-REFERENCE TO RELATED APPLICATIONS

This application claims the benefit of priority to U.S. Provisional Application No. 63/354,837, filed on Jun. 23, 2022. The aforementioned application is incorporated by reference herein in its entirety.

### STATEMENT REGARDING FEDERALLY SPONSORED RESEARCH OR DEVELOPMENT

This invention was made with government support under 8J-30009-0029A awarded by the Department of Energy. The government has certain rights in the invention.

### SEQUENCE LISTING

A Sequence Listing accompanies this application and is submitted as an XML file of the sequence listing named “702581_02355_sequence_listing.xml” which is 29,473 bytes in size and was created on Jun. 23, 2023. The sequence listing is electronically submitted via Patent Center with the application and is incorporated herein by reference in its entirety.

### BACKGROUND

Cell free biosensing is a rapidly growing technology that allows fast, cheap, and on-site detection of small molecule ligands. The core of cell free biosensing is allosteric transcription factors (aTFs) that bind DNA and control gene expression via physical interactions with small molecule ligands. Currently, to setup a new cell free biosensor, an aTF must be well characterized with respect to the operator sequence and the small molecule regulator. These are intensive and time-consuming experiments, blockading the expansion to sensing new molecules. Furthermore, aTFs of interest can be found in many different organisms and their natural mechanism may not translate to E. coli systems.

### SUMMARY

In an aspect, a method for generating novel nucleic acid sequences that bind to an allosteric transcription factor is provided. The method includes: (i) providing a library of partially randomized nucleic acid sequences, an allosteric transcription factor (aTF), and a ligand for the aTF; (ii) contacting the library of partially randomized nucleic acid sequences to the aTF in the presence of the ligand to produce one or more nucleic acid-aTF complexes and non-bound nucleic acid sequences; (iii) partitioning the nucleic acid-aTF complexes away from the non-bound nucleic acid sequences; (iv) purifying the nucleic acid sequences from the nucleic acid-aTF complexes to generate purified nucleic acid sequences; and (v) amplifying the purified nucleic acid sequences to generate amplified purified nucleic acid sequences.

In another aspect, a system for generating novel nucleic acid sequences that bind to an allosteric transcription factor (aTF) is provided, that includes a library of partially randomized nucleic acid sequences that comprise nucleic acid sequences that are similar to a native nucleic acid sequence that binds to an aTF but differ in at least one nucleic acid residue from the native nucleic acid sequence, an aTF, and a ligand to the aTF.

In yet another aspect, a nucleic acid sequence having the polynucleotide sequence of any one of SEQ ID NOs: 1, 4-23, or 25-34, or a polynucleotide sequence that is at least about 90% identical to any one of SEQ ID NOs: 1, 4-23, or 25-34 is provided.

In another aspect, a cell-free biosensor, kit, method, or system comprising a novel aTF nucleic acid binding sequence is provided.

### BRIEF DESCRIPTION OF THE FIGURES

FIG. 1A is a schematic depiction of a design of a cell-free operator selection assay.

FIG. 1B is a schematic depiction of the operator library design.

FIGS. 1C and 1D illustrate selected CadR operators identified in the selection assay to sense cadmium as compared to wild type (WT).

FIG. 1E depicts a bar graph illustrating the relative amounts of promoter recovery (in %) for each round of quantitative PCR (qPCR) in the selection assay for CadR.

FIG. 1F is a bar graph depicting one promoter identified in the selection assay showing the relative levels of reporter produced with and without cadmium.

FIG. 2 depicts the results of qPCR threshold values from four rounds of selection for 20 different allosteric transcription factors (aTFs). Lower threshold values indicate enrichment. Red line indicates initial aTF used in BLAST to find homologs as described in Example 2.

FIG. 3A is a schematic depiction of the selection assay used as described in Example 3.

FIG. 3B is a bar graph (left) showing the relative percent recovery of HosA promoter selection from the selection assay described in Example 3, and shows the validation of a selected promoter (right) showing activation in the presence of 4-hydroxy benzoic acid (4HB) as compared to a traditionally designed promoter (middle).

FIG. 3C illustrates the structures of various hydroxy benzoic acids similar to 4HB.

FIG. 3D depicts a sequence similarity network (SSN) of HosA homologs. HosA is indicated and all the dark blue circles were chosen for operator selection assays. 16 clusters are illustrated numbered 1-16 going from the left to the right and top to the bottom. The bottom row also depicts the singleton (24 total) homologs.

FIG. 3E are graphs for selected member(s) of each cluster of HosA homologs showing every qPCR round of operator selection with the Ct value from each round. High to low Ct value indicates library enrichment of aTF binding operators.

FIG. 3F depicts relative level of induction in sensing reactions with the HosA operator with 96 HosA homologs. A1-H12 represent distinct aTFs and each small square is buffer or a hydroxybenzoic acid, 2HB, 3HB, or 4HB.

FIG. 3G depicts relative level of induction in a sensing assay for 384 aTF/operator pairs using 1 mM 2HB, 3HB, and 4HB. Blue indicates OFF while white and red indicate and ON signal.

FIG. 3H depicts results of sensing assays for select aTF/operator pairs to analyze their selectivity to sense various mono-and di-substituted benzoic acids.

FIGS. 4A and 4B illustrate selected CadR operators identified in the selection assay to sense cadmium as compared to wild type (WT).

FIGS. 5 and 6 are tables listing various DNA sequences utilized in the Examples as described herein.

### DETAILED DESCRIPTION

### Overview

In various aspects, the methods and systems described herein can be used for generating novel nucleic acid sequences that bind to an allosteric transcription factor and novel nucleic acid sequences generated by said methods. In various aspects, the cell-free biosensor, kit, and systems are also described that utilize and/or include the novel nucleic acid sequences.

Transcription factors can function as natural sensors. For instance, allosteric transcription factors (aTFs) are proteins that respond to small molecule ligands to regulate gene expression. Employment of known aTFs as biosensors has proven valuable for controlling the overexpression of proteins, the flux of metabolites, sensing the presence of high value chemicals or toxins, and for high-throughput optimization of chemical synthesis. A major bottleneck for utilizing new aTFs is the discovery of an active operator sequence.

Biosensors can be utilized in numerous applications, including but not limited to biological systems engineering, medical diagnostics, contaminant and/or toxin detection, chemistry optimization, reaction optimization, and metabolic engineering.

One limitation to developing new aTFs as biosensors is that functional DNA components are often unknown. Another limitation is that predicting ligand response can be difficult. Currently, to setup a new cell free biosensor, an aTF must be well characterized with respect to the operator sequence and the small molecule regulator or ligand. These are intensive and time-consuming experiments, blockading the expansion to sensing new molecules. Furthermore, aTFs of interest can be found in many different organisms and their natural mechanism may not translate to E. coli systems. Cell-free systems can provide a platform to study new transcription factors and develop useful biosensors.

The present disclosure overcomes these limitations through the development of a selection assay that can select active operators for any given transcription factor, in various aspects. For instance, in various aspects, the methods and systems disclosed herein can include a cell-free operator selection assay to identify novel aTF nucleic acid binding sequences.

The methods disclosed herein are agnostic towards the natural genomic context by evolving a functional promoter in the background of highly active E. coli parts, leading to robust sensors. In various aspects, using plates (e.g., such as a 96-well plate format), 96different aTFs can be selected at a single time and 4-5 selection rounds can be performed in a single day. Utilizing the methods disclosed herein to identify new classes of aTFs and associated nucleic acid binding sequences will greatly expand the panel of transcription factors available for on-demand diagnostics.

The disclosed systems, methods, and compositions may be used for the following applications: high-throughput promoter discovery and characterization, rapid development of cell free biosensing circuits, rapid expansion of molecules that can be sensed, biosensing of water contaminates, biosensing of synthetic reaction products, biosensing of toxins, biosensing human metabolites, and synthetic biology applications.

The present invention is described herein using several definitions, as set forth below and throughout the application.

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, “partially randomized” refers to the property of a nucleic acid sequence to have some sequence similarity to a reference sequence, except that, at least one residue of the nucleic acid sequence is different as compared to the reference sequence. In some embodiments, partially randomized sequences comprise differences in at least 1, at least 2, at least 3, at least 4, at least 5, at least 6, at least 7, at least 8, at least 9 or at least 10 or more nucleic acid residues.

As used herein, “library of partially randomized sequences” refers to a library of sequences as known in the art, except that the sequences are generated to be different from a reference sequence, e.g., an allosteric transcription factor operator, in a calculated number of residues that are randomized. In other words, a library of partially randomized sequences comprises a wide variety of sequences that are based on the reference sequence to enable selection of alternative sequences to the reference sequence that binds to an aTF in complex with a ligand.

As used herein, a “ligand to an aTF” refers to any molecule that complexes and/or binds with an aTF, thereby allowing the aTF ligand complex to bind (or unbind) to a nucleic acid sequence, e.g., a metal ion, an organic compound, an inorganic compound, etc.

As used herein, “an allosteric transcription factor (aTF)” refers to proteins which, upon binding a small molecule such as a ligand to an aTF, undergo a conformational change that alters their affinity for an operator DNA sequence, e.g., HosA, which has the traditional ligand 4-hydroxybenzoic acid, or CadR, which has the traditional ligand of, e.g., cadmium.

As used herein, the terms “regulation” and “modulation” may be utilized interchangeably and may include “promotion” and “induction.” For example, a transcription factor that regulates or modulates expression of a target gene may promote and/or induce expression of the target gene. In addition, the terms “regulation” and “modulation” may be utilized interchangeably and may include “inhibition” and “reduction.” For example, a transcription factor that regulates or modulates expression of a target gene may inhibit and/or reduce expression of the target gene.

The term “transcription factor” refers to a protein that regulates transcription of another protein, typically by interacting by one or more cis-acting DNA sequence, e.g., an operator, in or near the promoter for the other protein. A transcription factor may increase expression or decrease expression depending upon whether the transcription factor is activated or deactivated. A transcription factor may become activated or deactivated by an interaction with another molecule (e.g., a ligand as described herein). Such transcription factors are termed allosteric transcription factors (aTFs).

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA.

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

The terms “nucleic acid” and “oligonucleotide,” as used herein, may refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979,Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp.

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

In certain aspects, the disclosed subject matter is associated in part with methods, devices, kits and components for cell-free protein synthesis. Cell-free protein synthesis (CFPS) is known and has been described in the art. (See, e.g., U.S. Pat. Nos. 6,548,276; 7,186,525; 8,734,856; 7,235,382; 7,273,615; 7,008,651; 6,994,986 7,312,049; 7,776,535; 7,817,794; 8,298,759; 8,715,958; 9,005,920; U.S. Publication No. 2014/0349353, U.S. Publication No. 2016/0060301, U.S. Publication No. 2018/0016612, and U.S. Publication No. 2018/0016614, the contents of which are incorporated herein by reference in their entireties). A “CFPS reaction mixture” typically contains a crude or partially-purified bacterial extract (as used herein the terms “extract” and “lysate” are used interchangeably), an RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. In these other aspects, the CFPS reaction mixture can also include a DNA-dependent RNA polymerase to direct transcription of an RNA translation template encoding the open reading frame. In these other aspects, additional NTP's and divalent cation cofactor can be included in the CFPS reaction mixture. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention. For example, the cellular transcription and translational machinery may be provided in a lysate from an engineered bacterial strain, or the transcription and translational machinery may be purified separately and reconstituted to defined concentrations. In some embodiments, a lysate may be from an engineered bacterial strain, and include cellular transcriptional and translational machinery, and may also include other as other cellular proteins.

The CFPS systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

A variety of methods exist for preparing an extract competent for cell-free protein synthesis, including U.S. patent application Ser. No. 14/213,390 to Michael C. Jewett et al., entitled METHODS FOR CELL-FREE PROTEIN SYNTHESIS, filed Mar. 14, 2014, and now published as U.S. Patent Application Publication No. 2014/0295492 on Oct. 2, 2014, and U.S. patent application Ser. No. 14/840,249 to Michael C. Jewett et al., entitled METHODS FOR IMPROVED IN VITRO PROTEIN SYNTHESIS WITH PROTEINS CONTAINING NON STANDARD AMINO ACIDS, filed Aug. 31, 2015, and now published as U.S. Patent Application Publication No. 2016/0060301, on Mar. 3, 2016, the contents of which are incorporated by reference.

The CFPS system may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

Altering the physicochemical environment of the CFPS reaction to better mimic the cytoplasm can improve protein synthesis activity. The following parameters can be considered alone or in combination with one or more other components to improve robust CFPS reaction platforms based upon crude cellular extracts (for examples, S12, S30 and S60extracts).

The temperature may be any temperature suitable for CFPS. Temperature may be in the general range from about 10° C. to about 40° C., including intermediate specific ranges within this general range, include from about 15° C. to about 35° C., from about 15° C. to about 30° C., form about 15° C. to about 25° C. In certain aspects, the reaction temperature can be about 15° C. about 16° C., about 17° C., about 18° C., about 19° C., about 20° C., about 21° C., about 22° C., about 23° C., about 24° C., about 25° C.

The CFPS reaction can include any organic anion suitable for CFPS. In certain aspects, the organic anions can be glutamate, acetate, among others. In certain aspects, the concentration for the organic anions is independently in the general range from about 0 mM to about 200 mM, including intermediate specific values within this general range, such as about 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM and about 200 mM, among others.

The CFPS reaction can also include any halide anion suitable for CFPS. In certain aspects the halide anion can be chloride, bromide, iodide, among others. A preferred halide anion is chloride. Generally, the concentration of halide anions, if present in the reaction, is within the general range from about 0 mM to about 200 mM, including intermediate specific values within this general range, such as those disclosed for organic anions generally herein.

The CFPS reaction may also include any organic cation suitable for CFPS. In certain aspects, the organic cation can be a polyamine, such as spermidine or putrescine, among others. Preferably polyamines are present in the CFPS reaction. In certain aspects, the concentration of organic cations in the reaction can be in the general about 0 mM to about 3 mM, about 0.5 mM to about 2.5 mM, about 1 mM to about 2 mM. In certain aspects, more than one organic cation can be present.

The CFPS reaction can include any inorganic cation suitable for CFPS. For example, suitable inorganic cations can include monovalent cations, such as sodium, potassium, lithium, among others; and divalent cations, such as magnesium, calcium, manganese, among others. In certain aspects, the inorganic cation is magnesium. In such aspects, the magnesium concentration can be within the general range from about 1 mM to about 50 mM, including intermediate specific values within this general range, such as about 1 mM, about 2 mM, about 3 mM, about 5 mM, about 6 mM, about 7 mM, about 8 mM, about 9 mM, about 10 mM, among others. In preferred aspects, the concentration of inorganic cations can be within the specific range from about 4 mM to about 9 mM and more preferably, within the range from about 5 mM to about 7 mM.

The CFPS reaction includes NTPs. In certain aspects, the reaction use ATP, GTP, CTP, and UTP. In certain aspects, the concentration of individual NTPs is within the range from about 0.1 mM to about 2 mM.

The CFPS reaction can also include any alcohol suitable for CFPS. In certain aspects, the alcohol may be a polyol, and more specifically glycerol. In certain aspects the alcohol is between the general range from about 0% (v/v) to about 25% (v/v), including specific intermediate values of about 5% (v/v), about 10% (v/v) and about 15% (v/v), and about 20% (v/v), among others.

### Methods

In various aspects, as discussed above, the methods disclosed herein can identify novel nucleic acid sequences for aTF binding, which can be utilized as part of biosensor system.

In various aspects, the methods can include providing a library of partially randomized nucleic acid sequences, an allosteric transcription factor (aTF), and a ligand to the aTF. In various aspects, the partially randomized nucleic acid sequences can be formed using any convenient technique.

In various aspects, each of the library of partially randomized nucleic acid sequences can be present in a known promoter. For instance, in one example aspect, the library can include all or part of the E. coli promoter J23119 with a number of randomized base pairs in front of the −10 site. In various aspects, the number of randomized base pairs, e.g., in front of the −10 site of the J23119 promoter can be in a range of 10-40 or 15-35, or 17-30, or about 17, about 20, about 25, about 27, or about 30. In one or more aspects, the library of partially randomized nucleic acid sequences can include nucleic acid sequences about 98% similar, about 95% similar, about 90% similar, about 85% similar, about 80% similar, about 75% similar, or about 70% similar to a native operator sequence that is recognized by the aTF.

As discussed above, the aTF can be any class and/or type of aTF. In various aspects, a non-limiting list of example aTFs includes CadR, HosA, Arac, ArsR, SriR/GutR, NanR, AllR, GylR3, TraR, BenM, MhqR, EmrR, Sav_4189, TcaR, BmrR, Betl, CamR, RamR, TetR, TtgR, and MphR. In various aspects, the ligands for certain aTFs are known, e.g., cadmium for CadR, 4-hydroxy benzoic acid (4HB) for HosA, and the ligands and associated aTFs listed in Table 3 herein. In alternative aspects, novel ligands or chemical variants of the known ligand can be used as the ligand for any aTF. As one example with respect to HosA, 2-hydroxy benzoic acid (2HB), 3-hydroxy benzoic acid (3HB), or other hydroxy benzoic acids can be used instead of, or in addition to, 4HB. In one aspect, when using alternative and/or novel ligands, the methods herein can be used to identify novel nuclide acid sequences that allow an aTF to function, e.g., biosensing function, in the presence of such novel or alternative ligands. One example of alternative ligands and associated novel sensing function is described in the Examples.

The aTF can be produced in any convenient manner. In one or more aspects, the aTF can be produced in a cell free protein synthesis system (CFPS). In alternative aspects, the aTF can be produced in a cell-based expression system. In various aspects, the aTF can include one or more affinity and/or purification tags, such as for example, biotinylation, FLAG tag, 6×HIS tag, and the like. In one example aspects, a biotinylated aTF is produced in a CFPS.

In various aspects, the methods can include contacting the aTF to the library of randomized nucleic acid sequences in the presence of the ligand. In one or more aspects, contacting the aTF to the library of randomized nucleic acid sequences in the presence of the ligand can be done in any convenient manner. In one aspect, the library of randomized nucleic acid sequences is mixed with the aTF in the presence of the ligand. In the same or alternative aspects, the aTF may be immobilized. For instance, the aTF may include an affinity and/or purification tag and may be immobilized via a respective affinity bead or surface. In one example aspects, a biotinylated aTF may be bound to streptavidin magnetic beads when exposed to the library of randomized nucleic acid sequences in the presence of the ligand. Exposing the aTF to the library of randomized nucleic acid sequences can be performed under any conditions suitable to allow for binding of the aTF to one or more of the library of randomized nucleic acid sequences in the presence of the ligand.

In various aspects, the nucleic acid-aTF complexes are separated from the non-bound nucleic acid sequences from the library using any suitable separation technique. In one or more aspects, the nucleic acid-aTF complexes are partitioned from the non-bound nucleic acids, e.g., using an affinity purification tag or epitope associated with the aTF. For example, exposing the nucleic acid-aTF complexes to magnetic bead separation/purification where the aTF is biotinylated and the magnetic beads comprise streptavidin, and then washing the bound complexes with buffer to remove the non-bound nucleic acids. One of skill in the art will understand that to facilitate the partitioning of nucleic acid sequences and/or nucleic acid-aTF complexes in the disclosed systems and methods, one or more component of the system or method, e.g., the aTF, the ligand, the nucleic acid sequences, may, in some aspects, comprise a label and can be separated using respective techniques associated with such a label. Example labels include, but are not limited to, an epitope recognized by an antibody, a protein, a nucleic acid sequence, biotin, or streptavidin, etc.

In various aspects, the nucleic acid sequences bound in the nucleic acid-aTF complexes can be purified to generate purified nucleic acid sequences. The nucleic acid sequences can be purified in any suitable manner. In one example aspect, the nucleic acid-aTF complexes can be exposed to one or more changes in buffer, salt concentration, or pH, which may release the nucleic acid from the complex, thereby allowing separation of the nucleic acid from the aTF, e.g., by using the affinity separation techniques mentioned above.

In various aspects, the purified nucleic acid sequences can be amplified to generate amplified purified nucleic acid sequences. Any suitable amplification technique can be used. In one example aspect, polymerase chain reaction (PCR) is utilized to amplify the purified nucleic acid sequences.

In various aspects, the selection assay described above is repeated, one more time, two more times, three more times, four more times, or five more times, to further enrich the nucleic acid sequences that bind to the aTF of interest. For example, in various aspects, the purified nucleic acid sequences can be exposed to an aTF in the presence of ligand, and the nucleic acid-aTF complexes can be separated from the non-bound nucleic acid sequences, then the bound or complexed nucleic acid sequences can then be separated from the aTF and amplified and/or used in downstream tests.

In various aspects, the nucleic acid sequences of interest can be sequenced using suitable technique, including but not limited to next generation sequencing (NGS).

In various aspects, the nucleic acid sequences of interest can be tested in a sensing assay or reaction to determine their biosensing ability. Example aspects for a sensing assay are described in the below Examples. In one example aspect, the nucleic acid sequences of interest can be present upstream of a reporter gene in a reporter construct. In such an example aspect, a CFPS system can be utilized for the sensing reaction or assay. For example, the reporter construct, a construct encoding for the aTF, and ligand are exposed to the CFPS reaction mixture, which can include E. coli extract, DNA, INTPs, salts, cofactors, amino acids, and then expression of the reporter gene can be assayed using suitable techniques. In certain aspects, the ligand may bind to the aTF and the reporter gene is expressed, whereas in the absence of the ligand, expression of the reporter gene is inhibited.

In various aspects, the methods can include using SELEX (systematic evolution of ligands by exponential enrichment) to evolve active operator sequences for diverse transcription factors in the background of an active E. coli promoter. In an example aspect, the selection assay can start by using cell free protein synthesis (CFPS) to make biotinylated aTFs that are subsequently immobilized on streptavidin magnetic beads. A library containing all or parts of the strong E. coli promoter J23119 with 17-20 “N” base pairs in front of the −10 site is incubated with the immobilized aTF, unbound DNA is washed away, and bound DNA is PCR amplified for ensuing rounds, in an example aspect. After 4-5 rounds of selection, enriched promoters are analyzed by next generation sequencing (NGS) and promoters counted, in an example aspect. The most enriched promoters are tested for sensing activity, in an example aspect.

### Cell-Free Biosensor, Kits, Nucleic Acids, and Systems

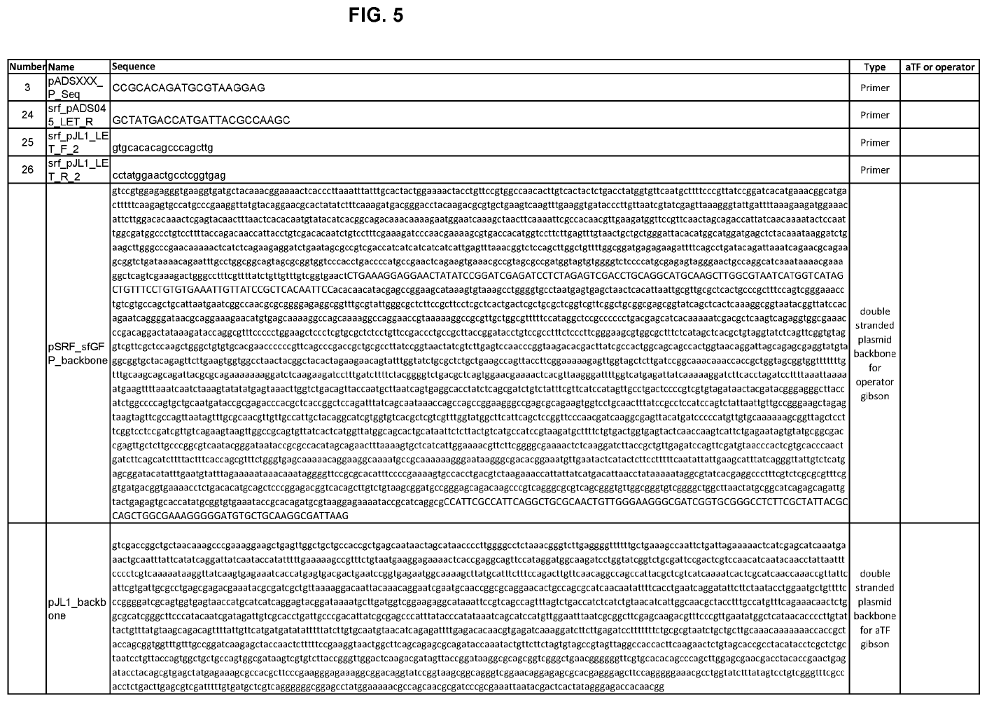

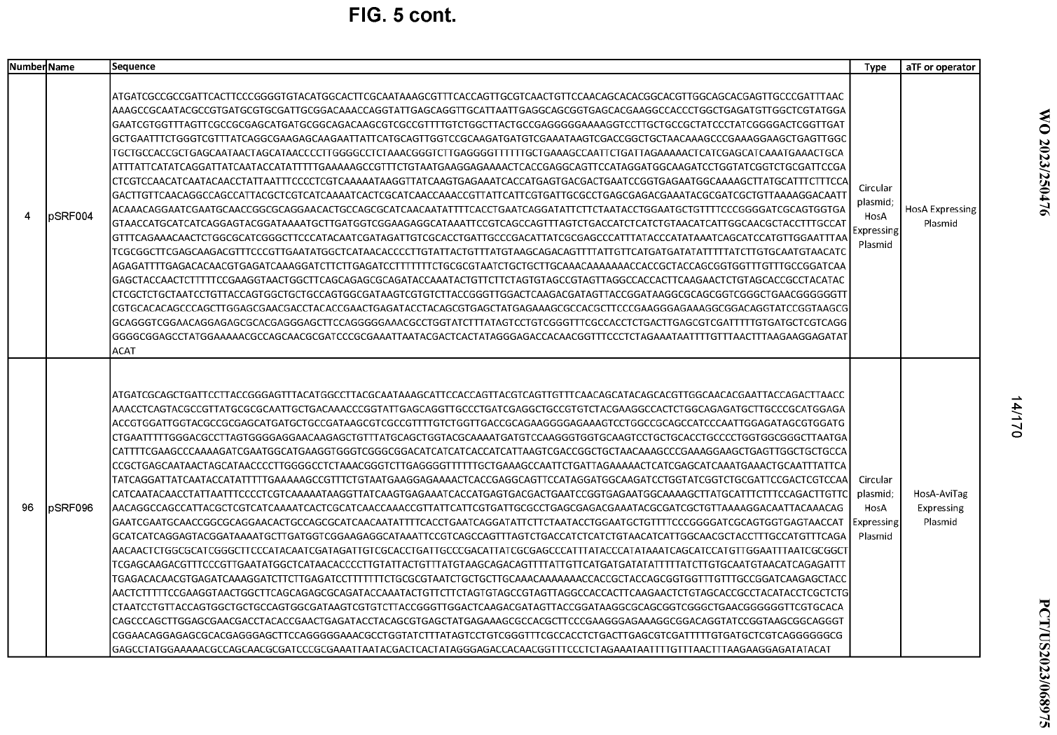

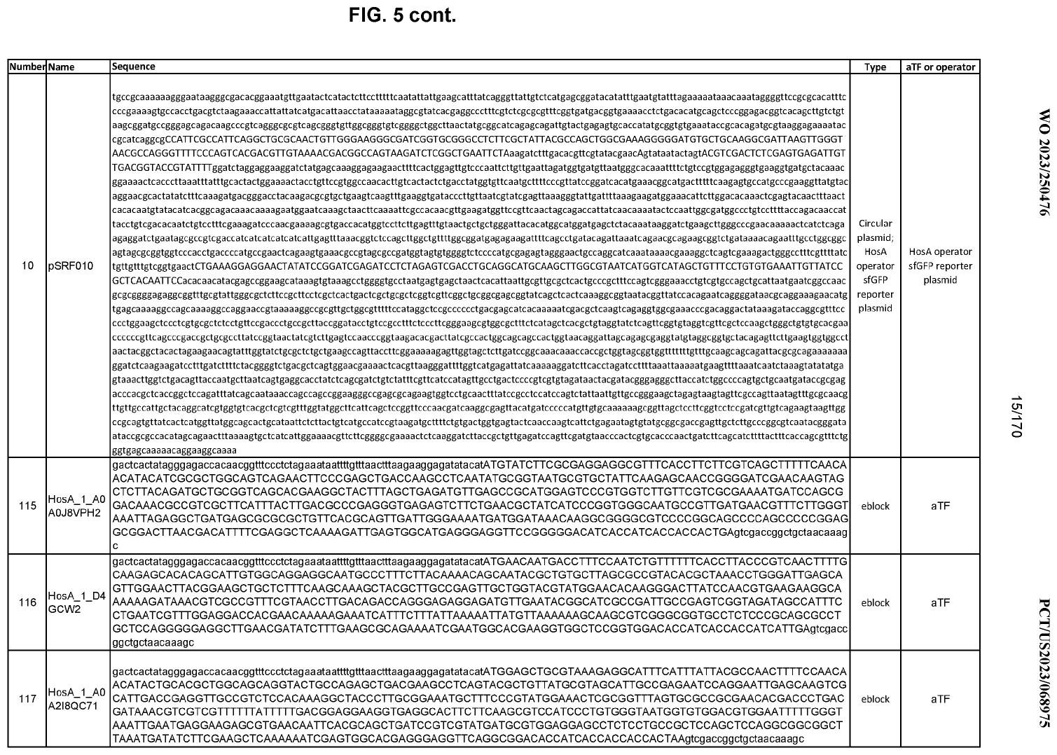

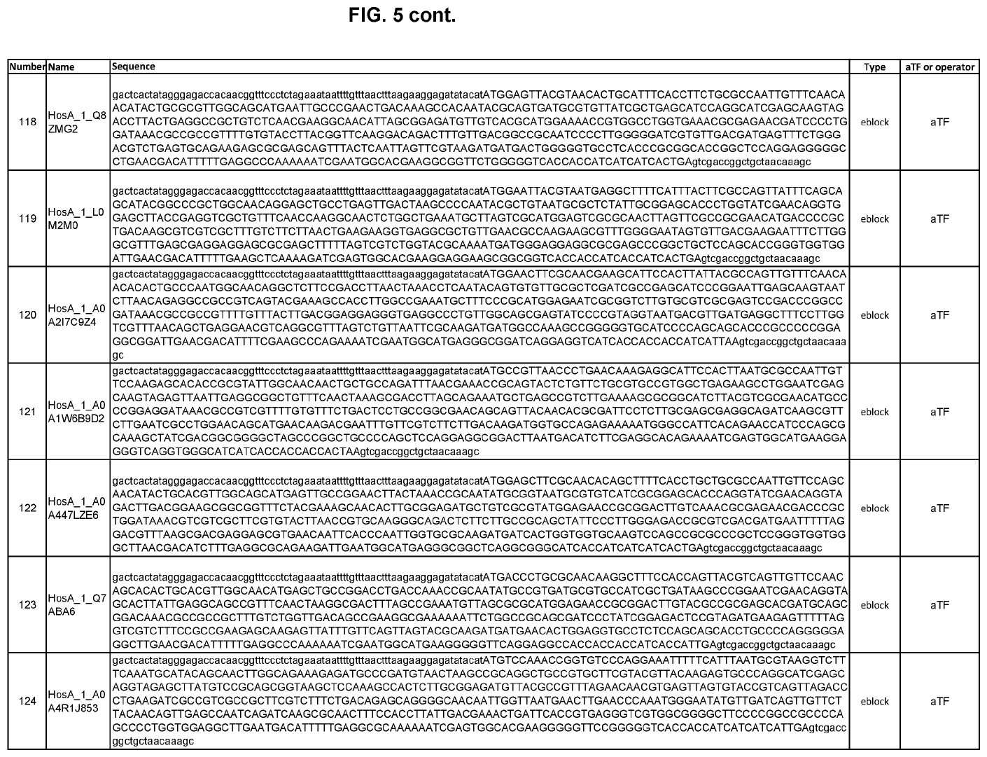

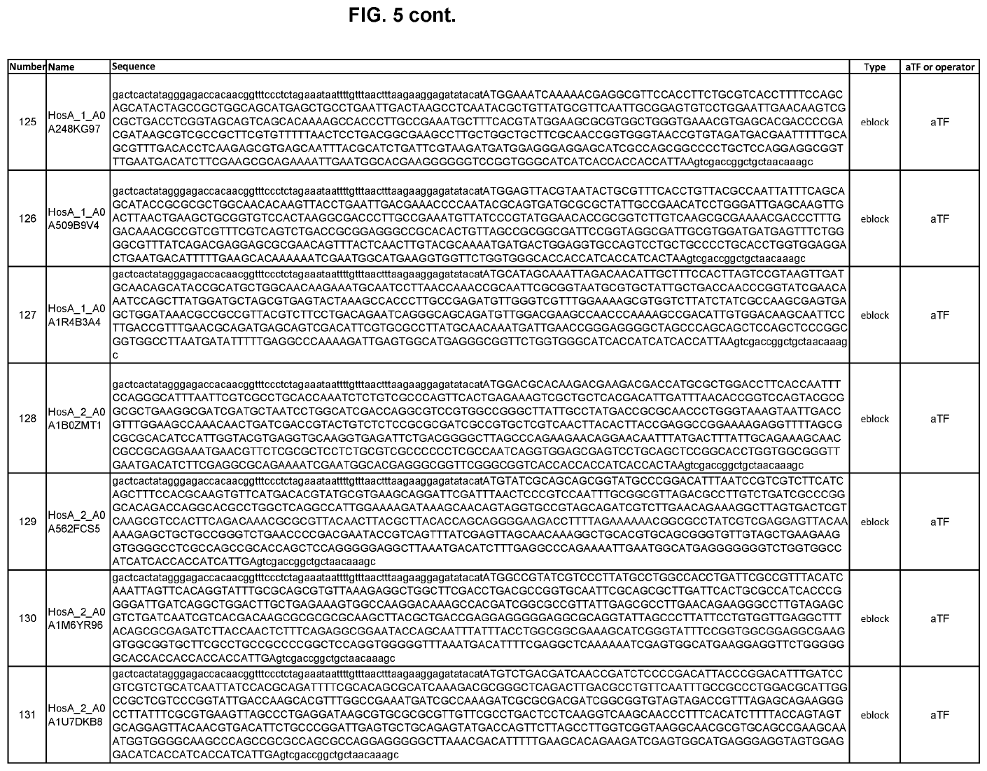

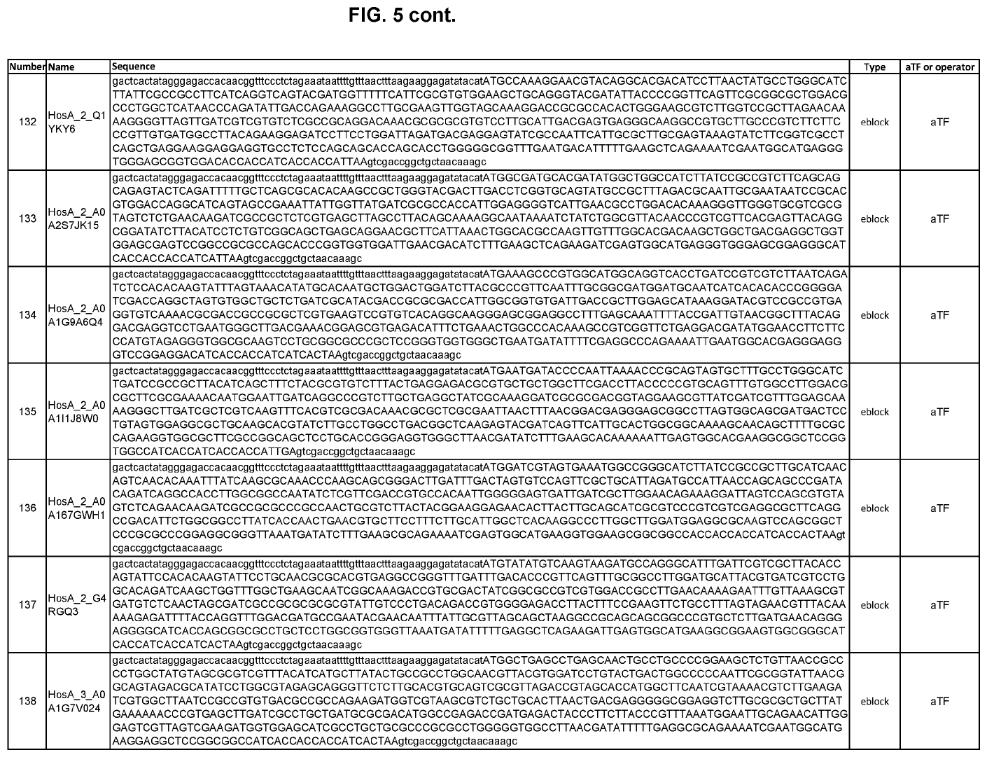

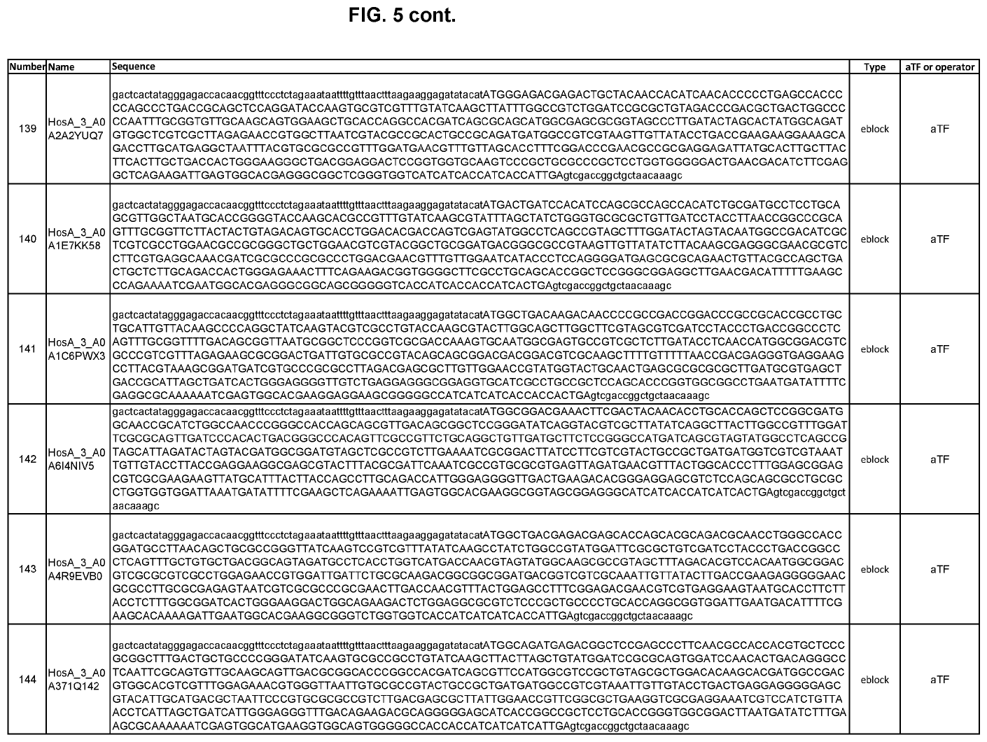

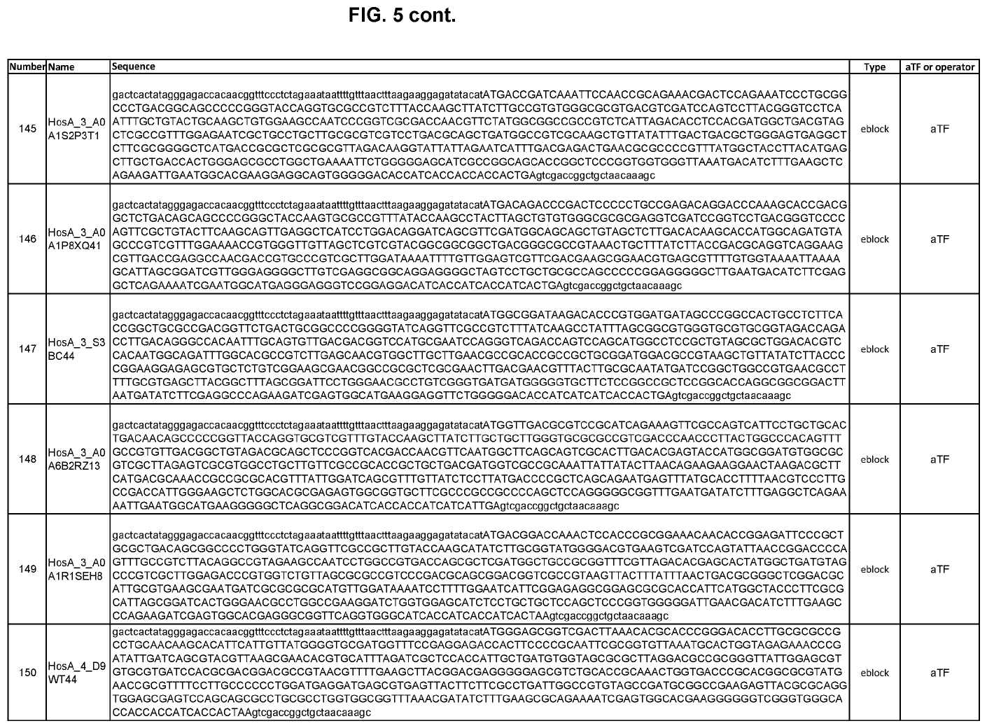

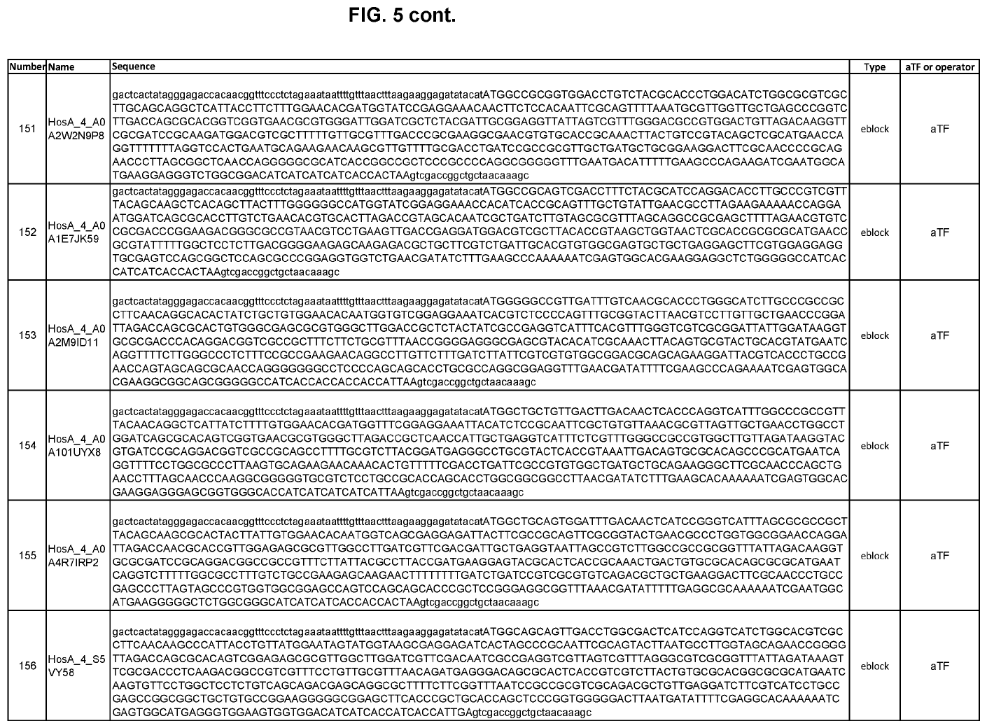

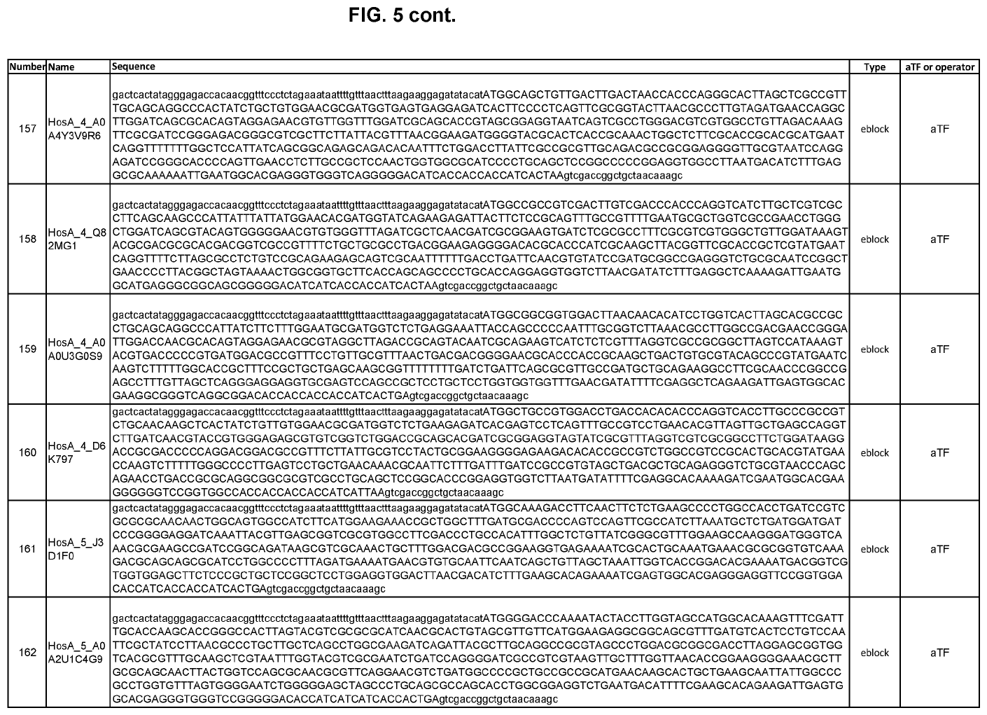

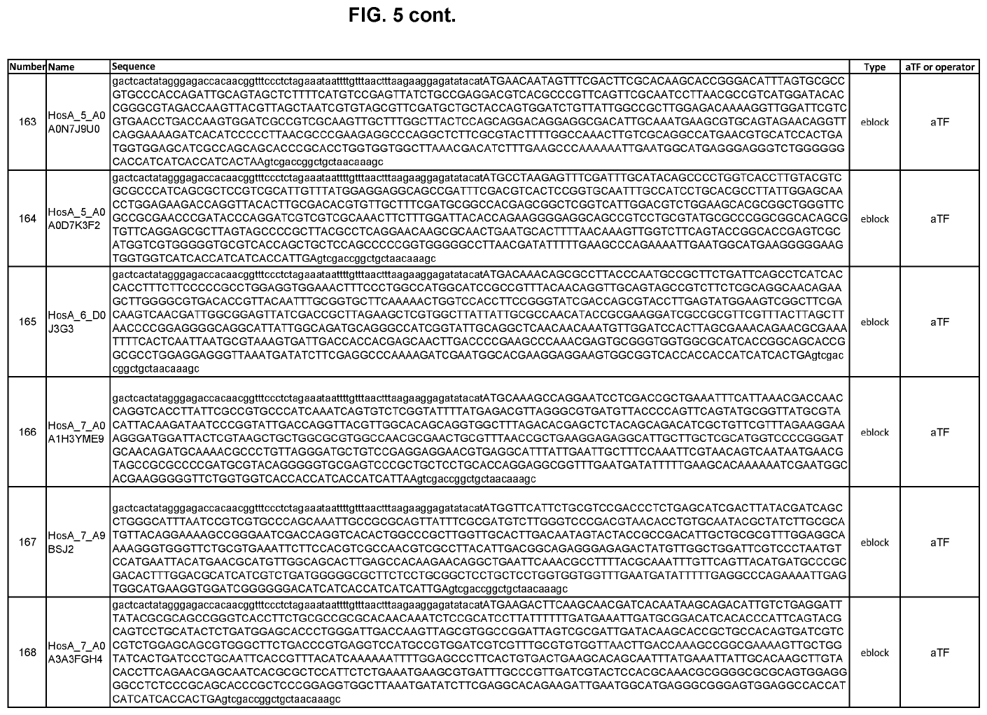

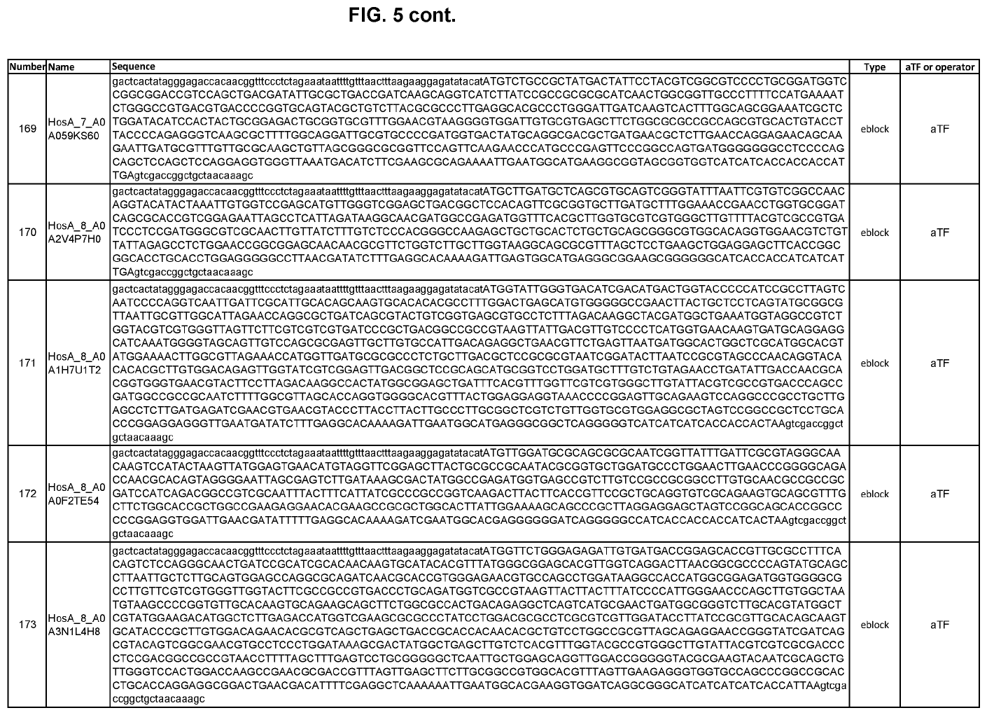

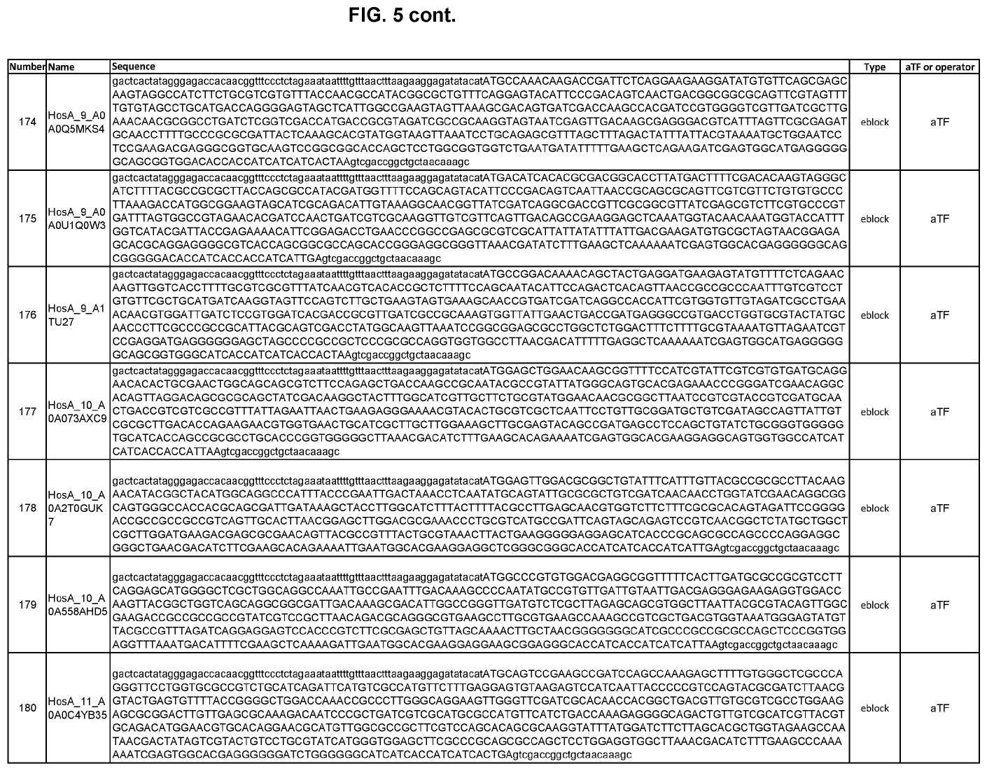

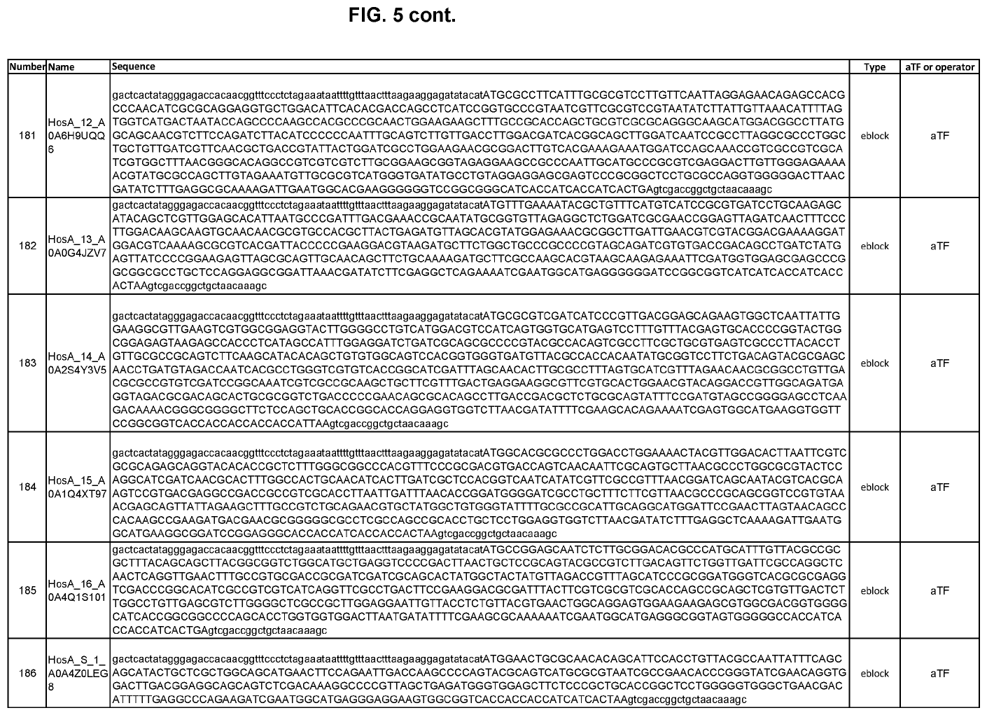

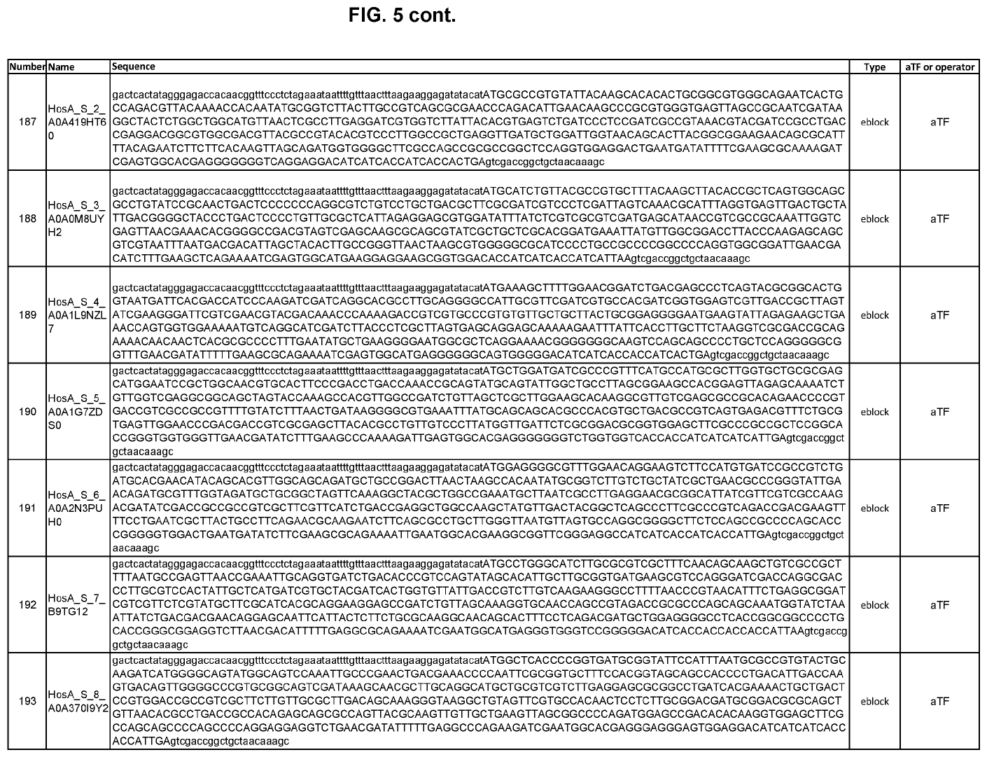

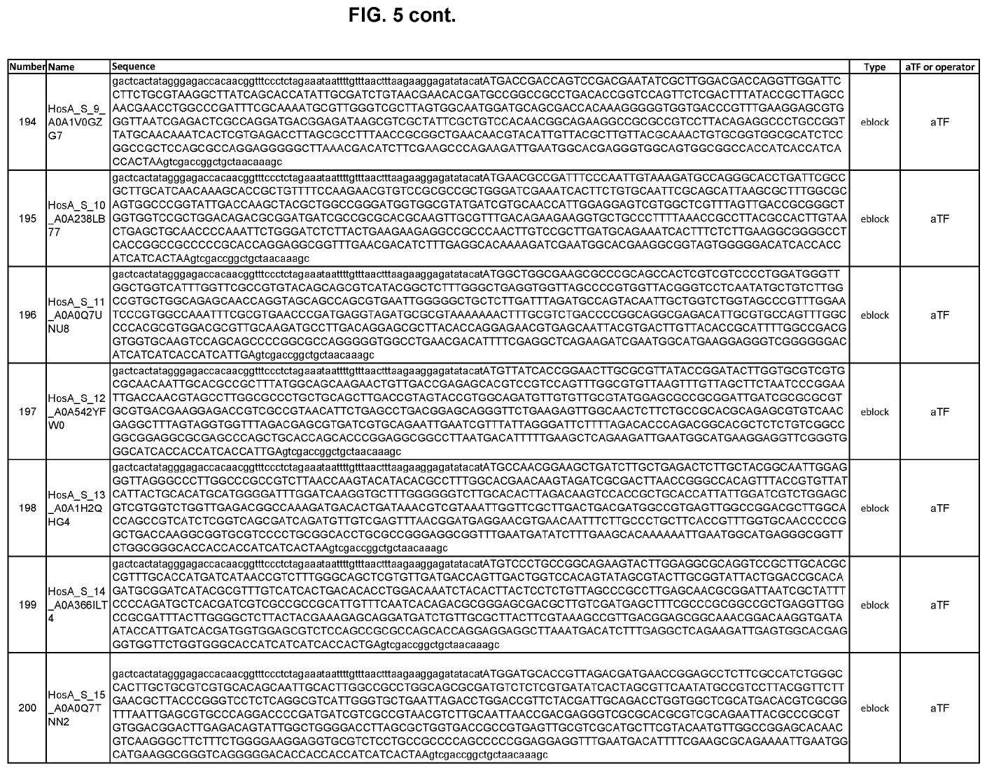

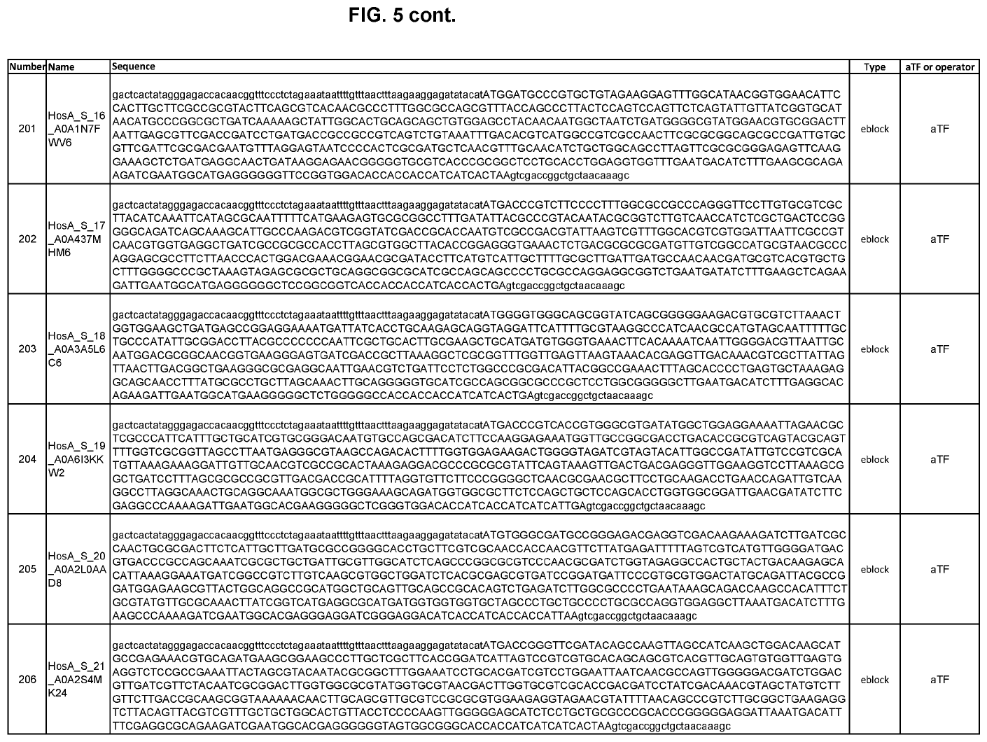

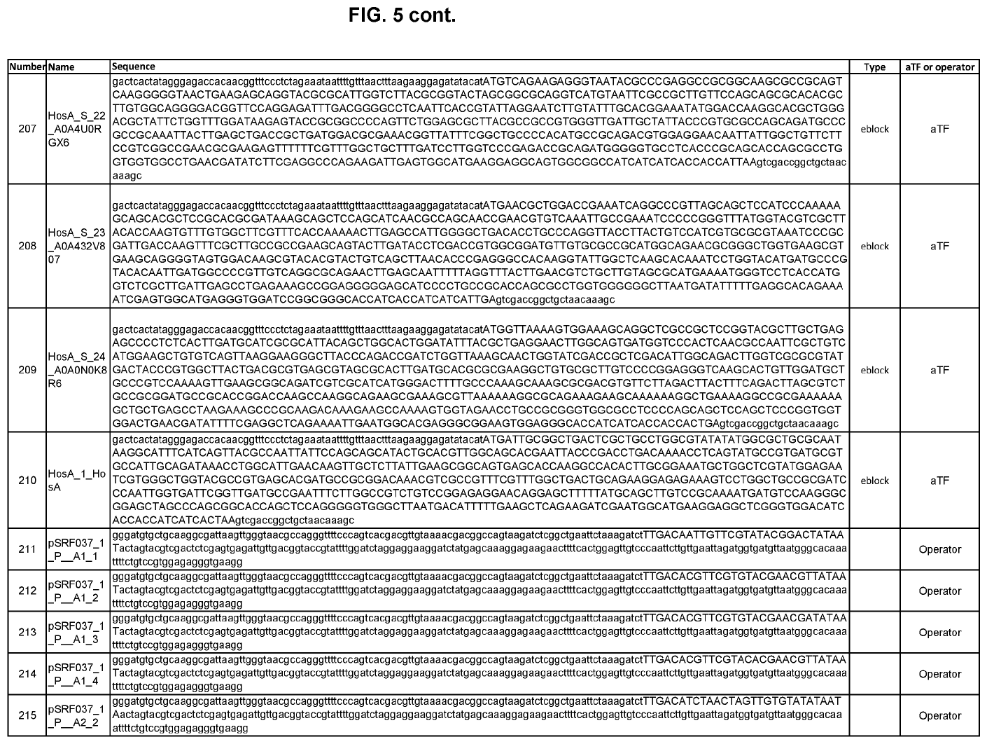

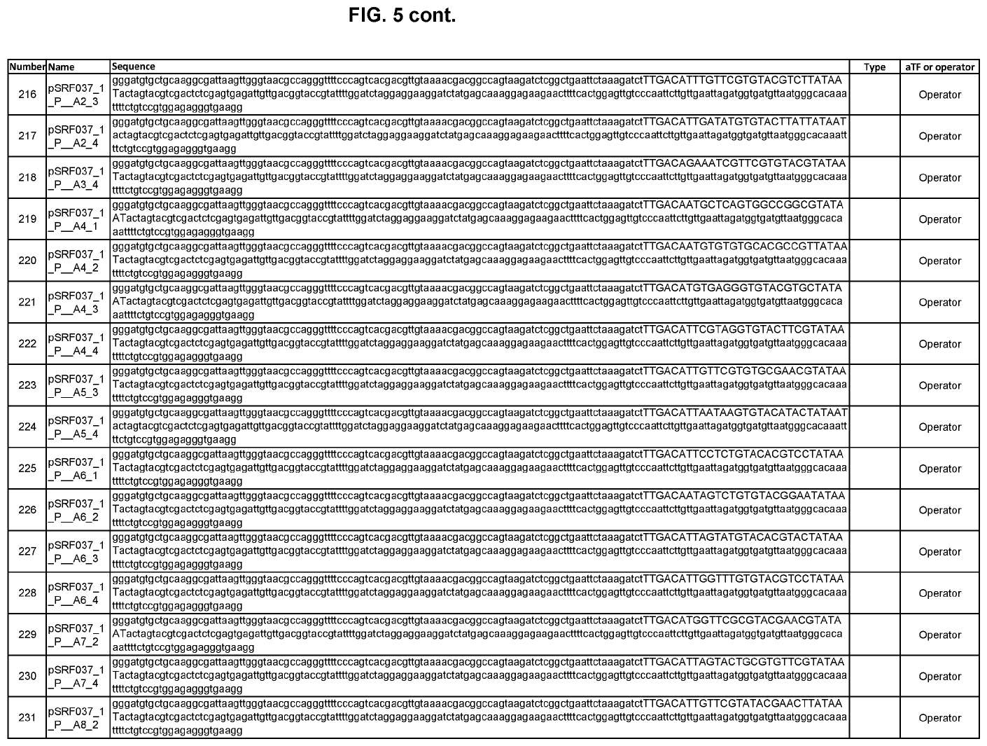

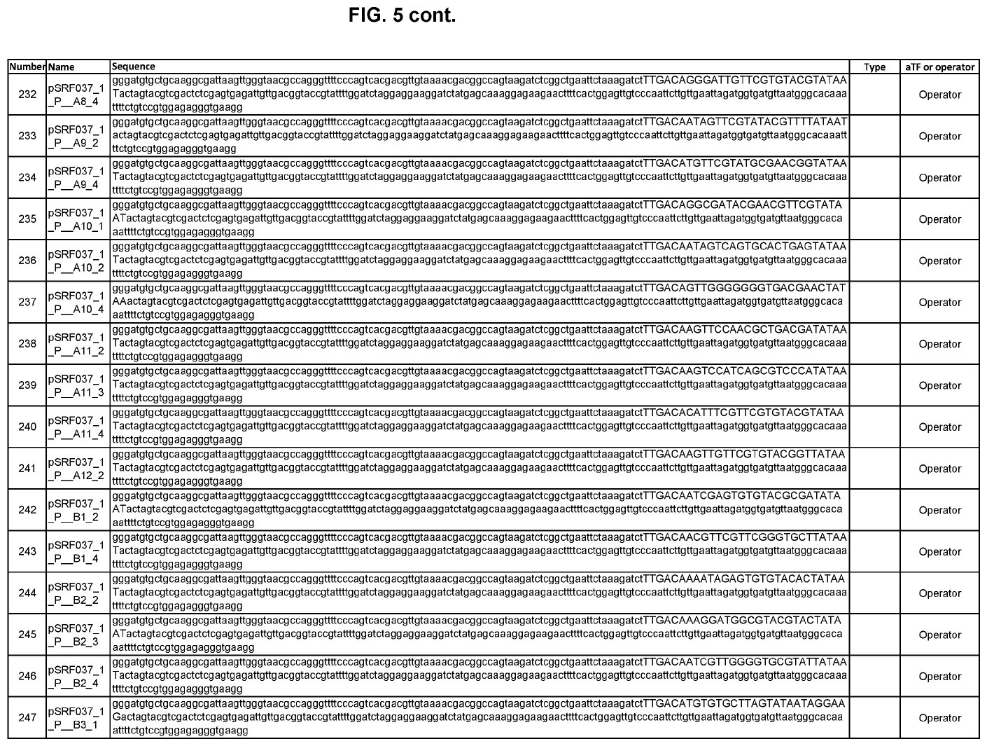

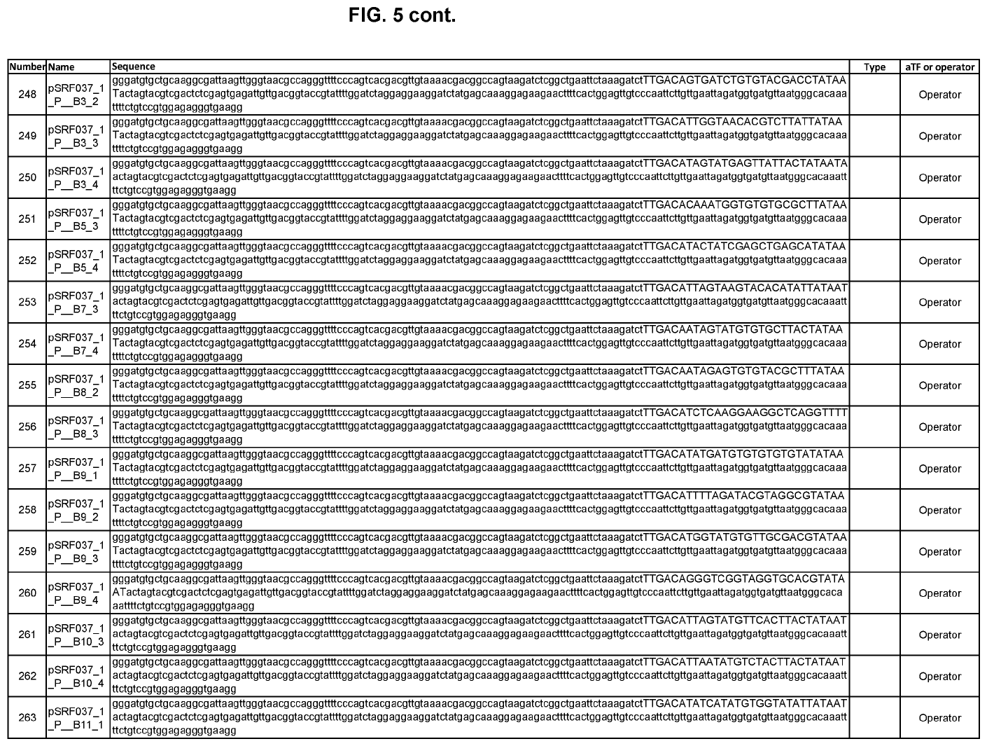

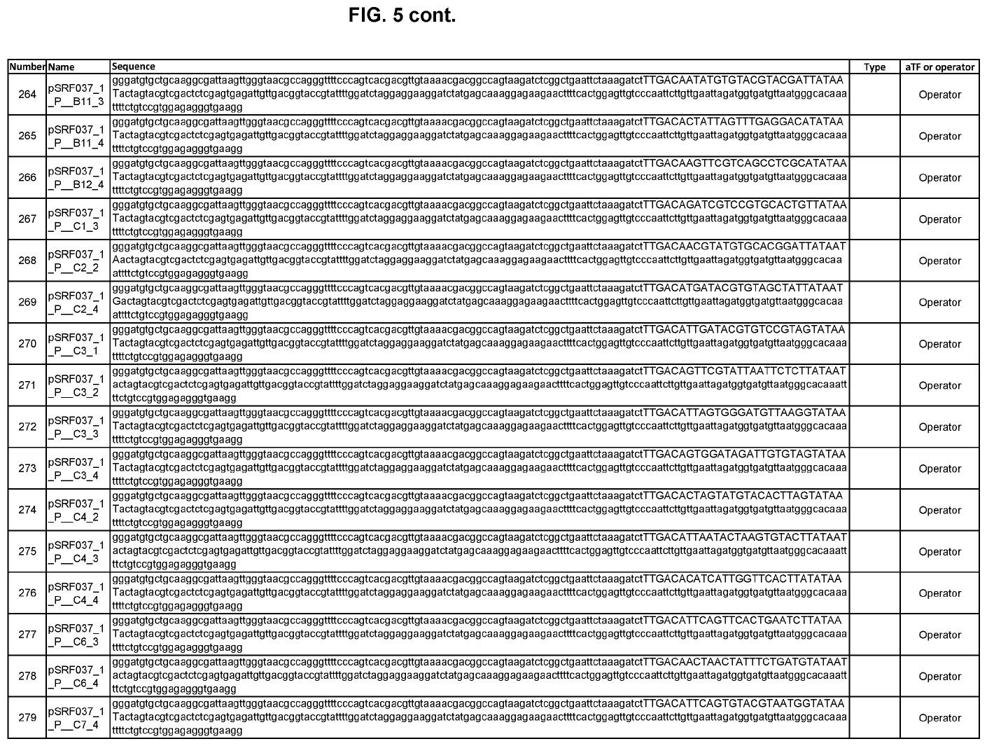

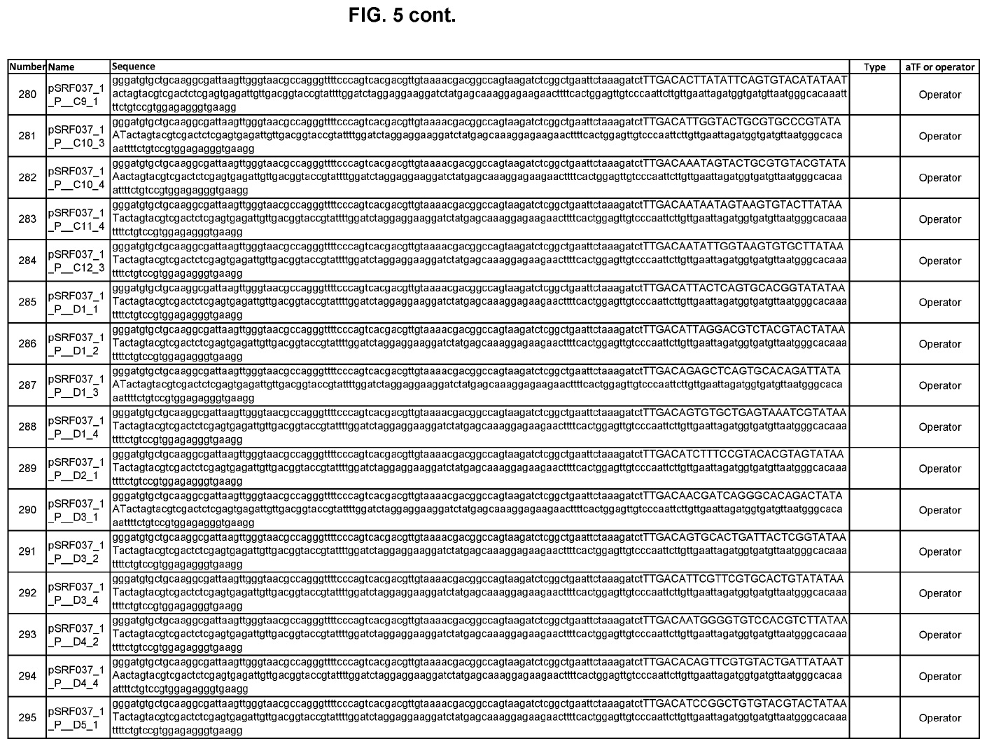

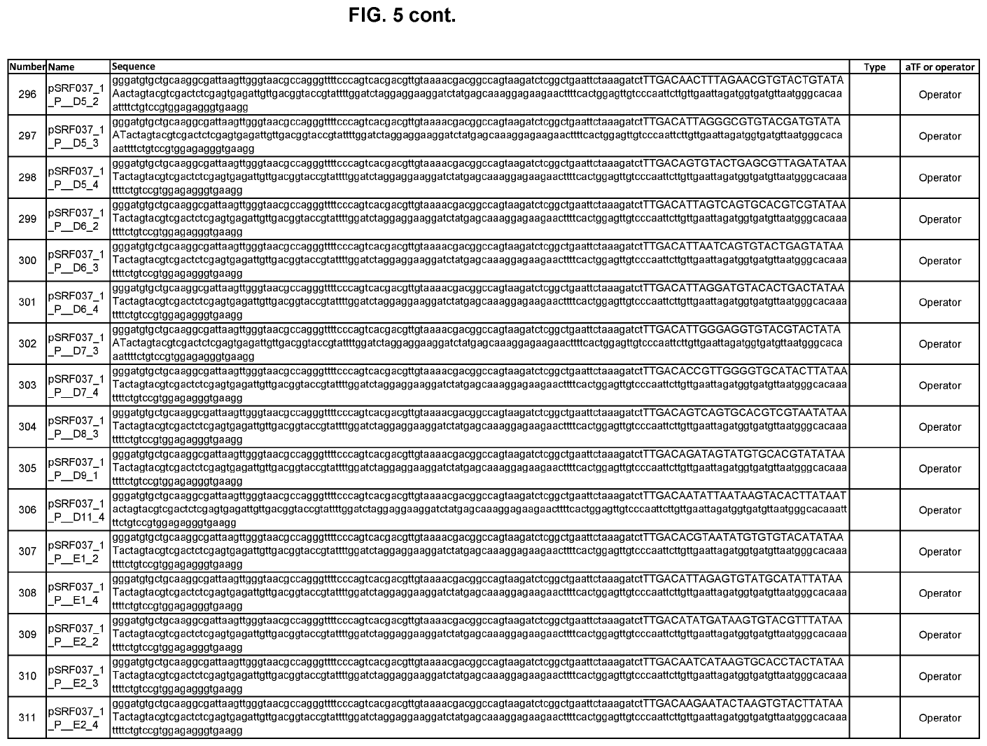

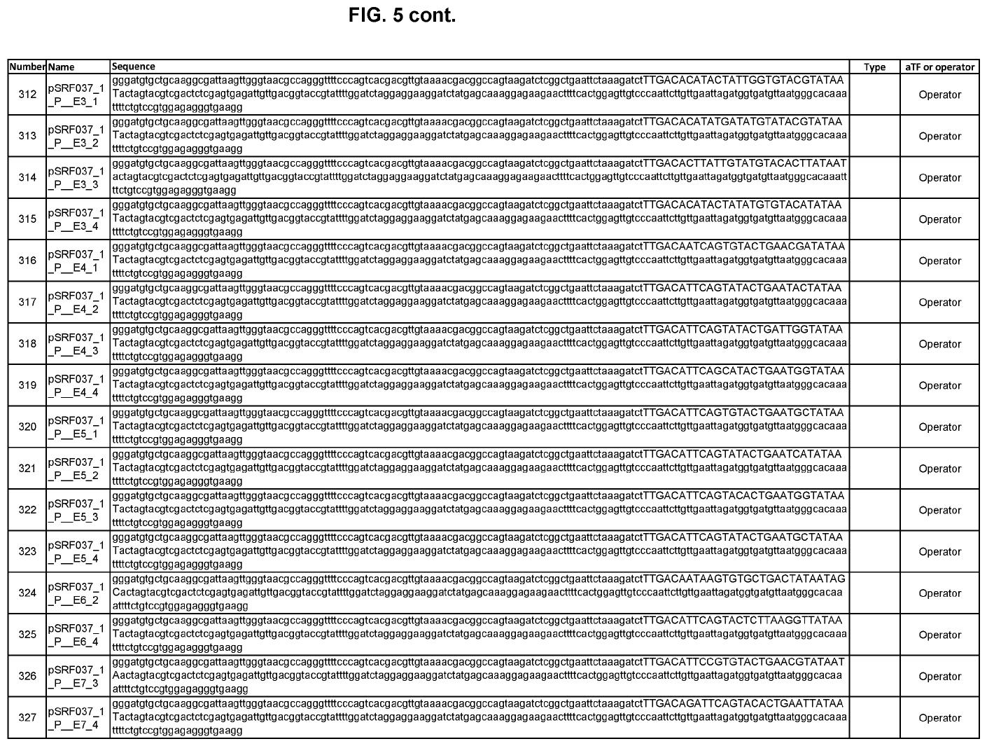

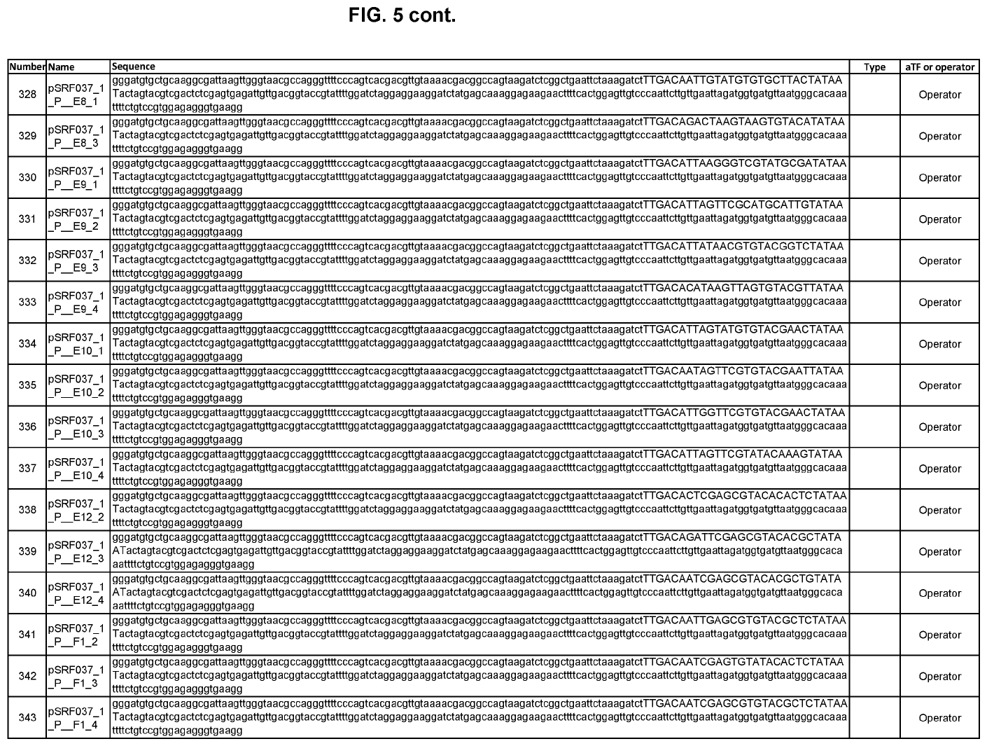

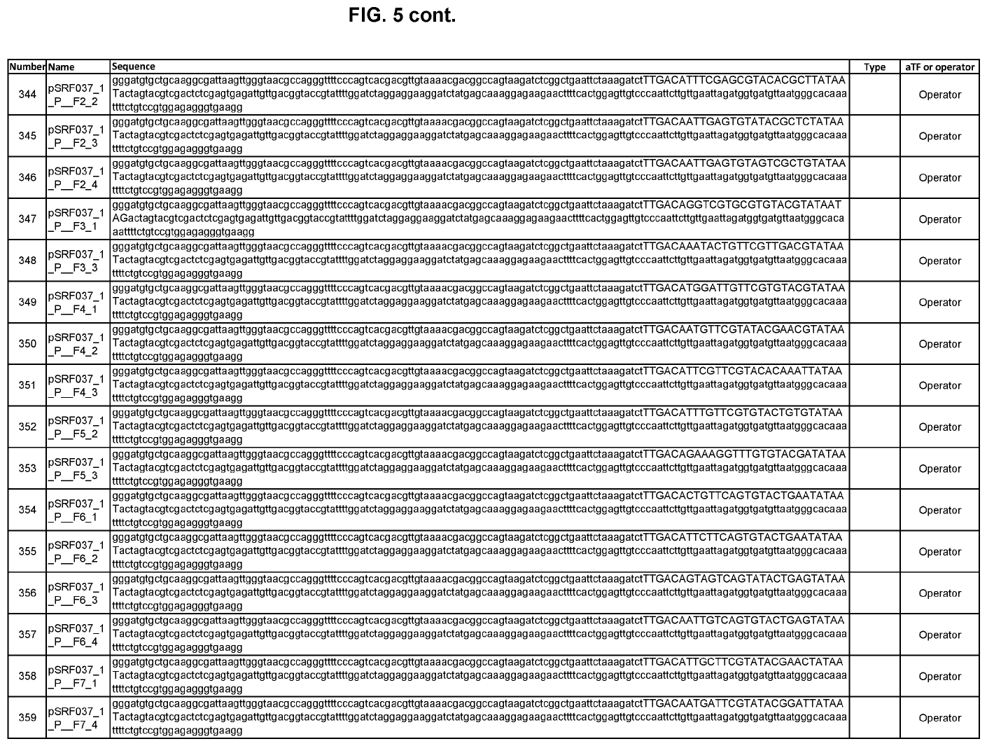

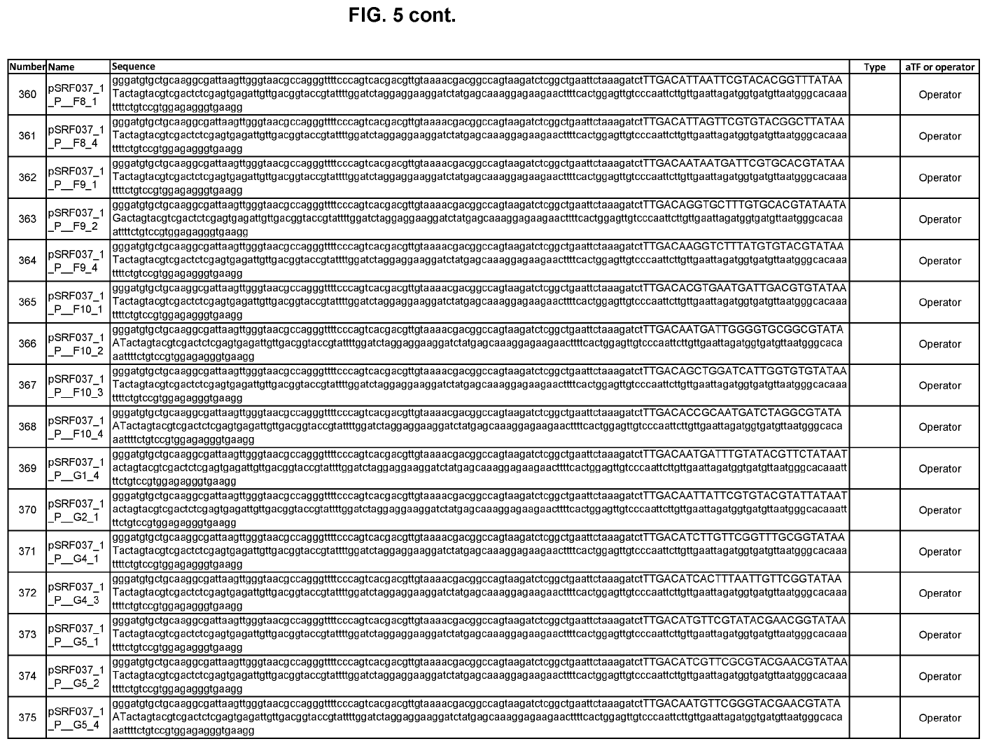

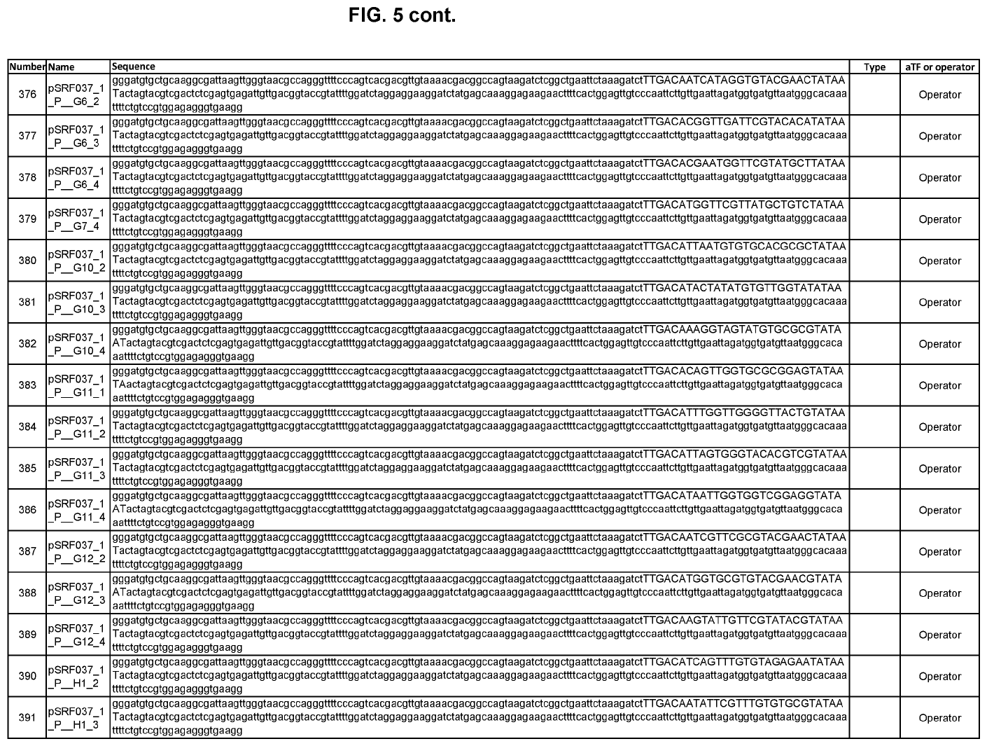

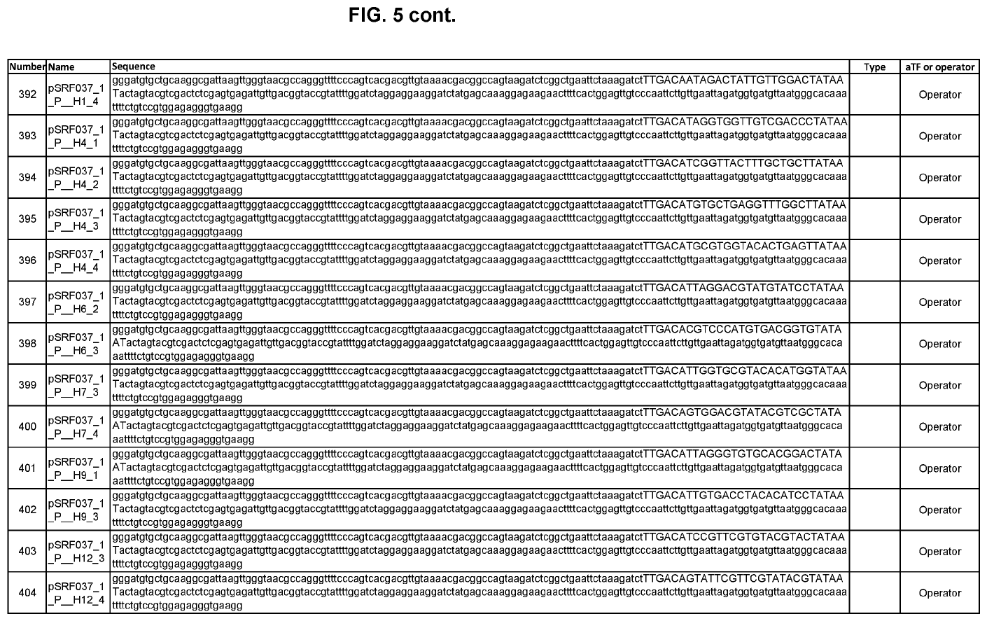

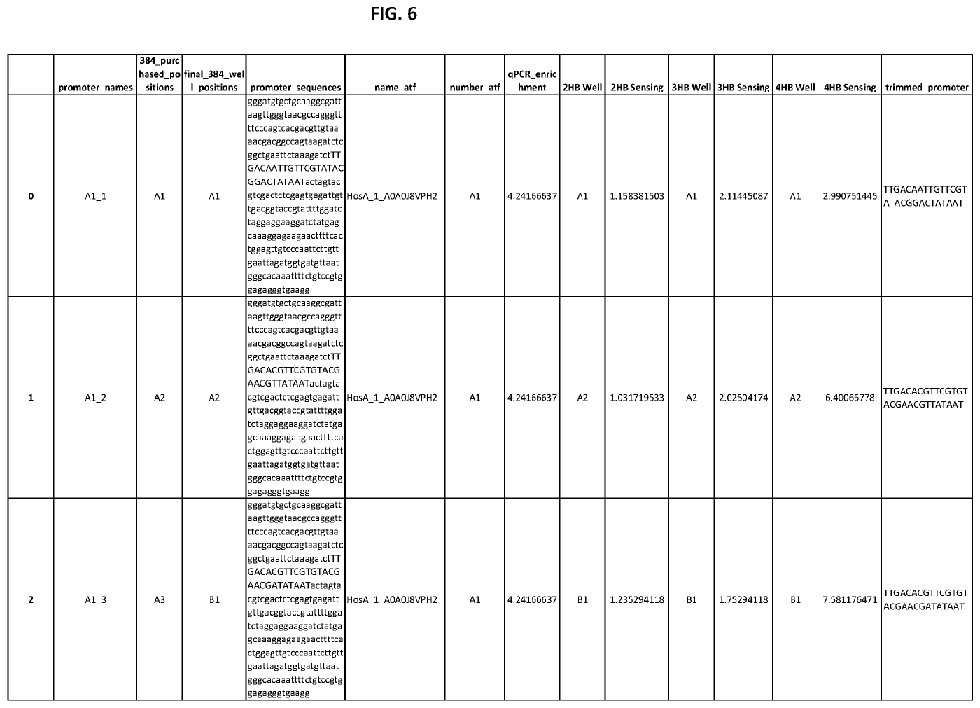

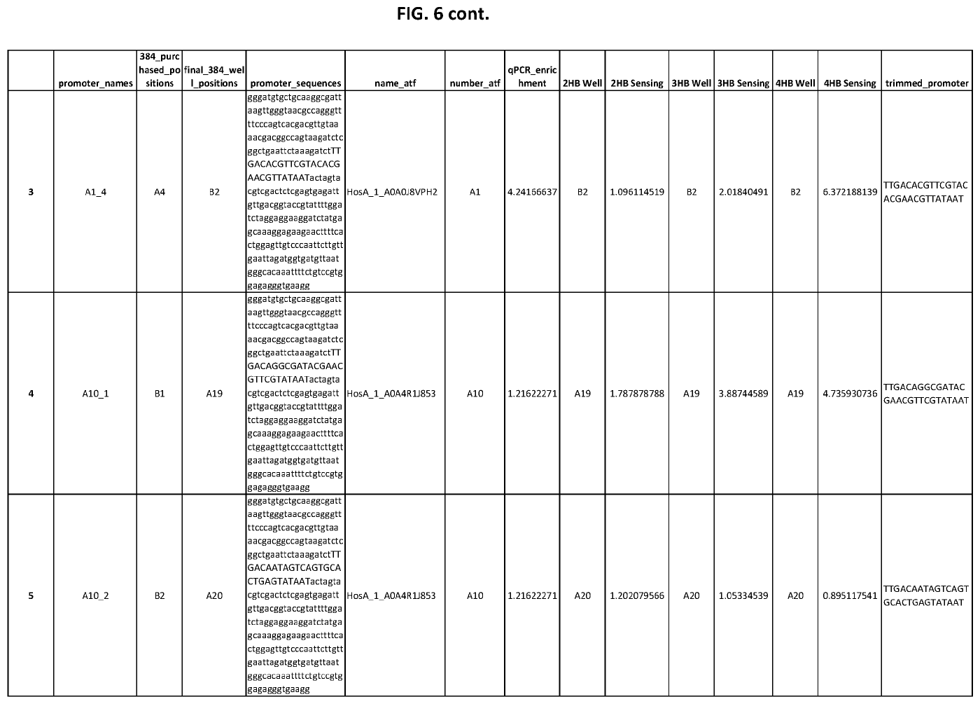

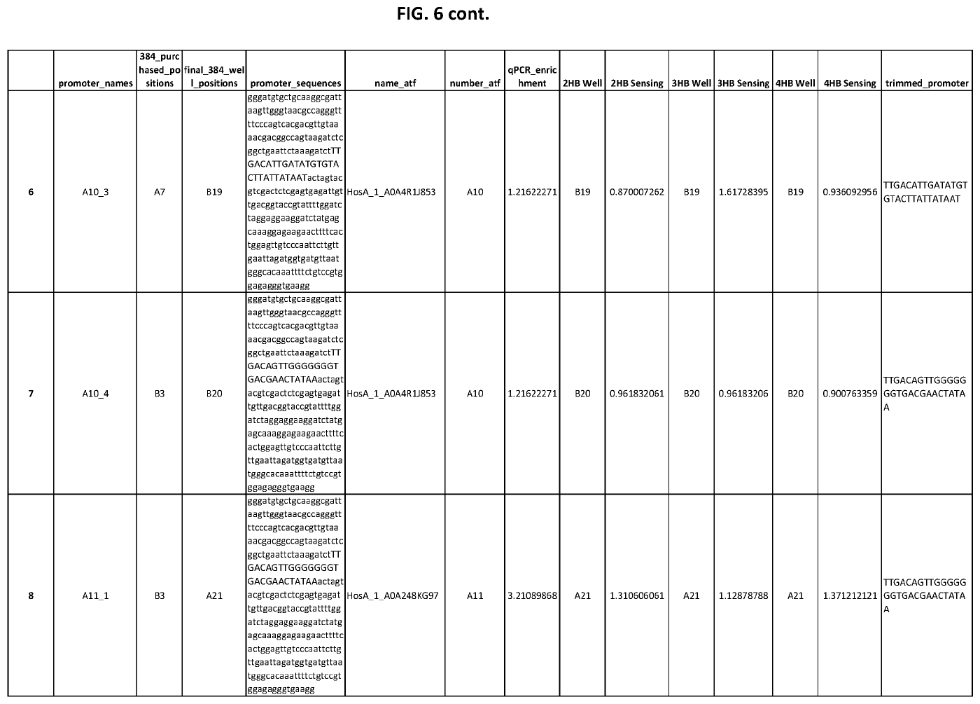

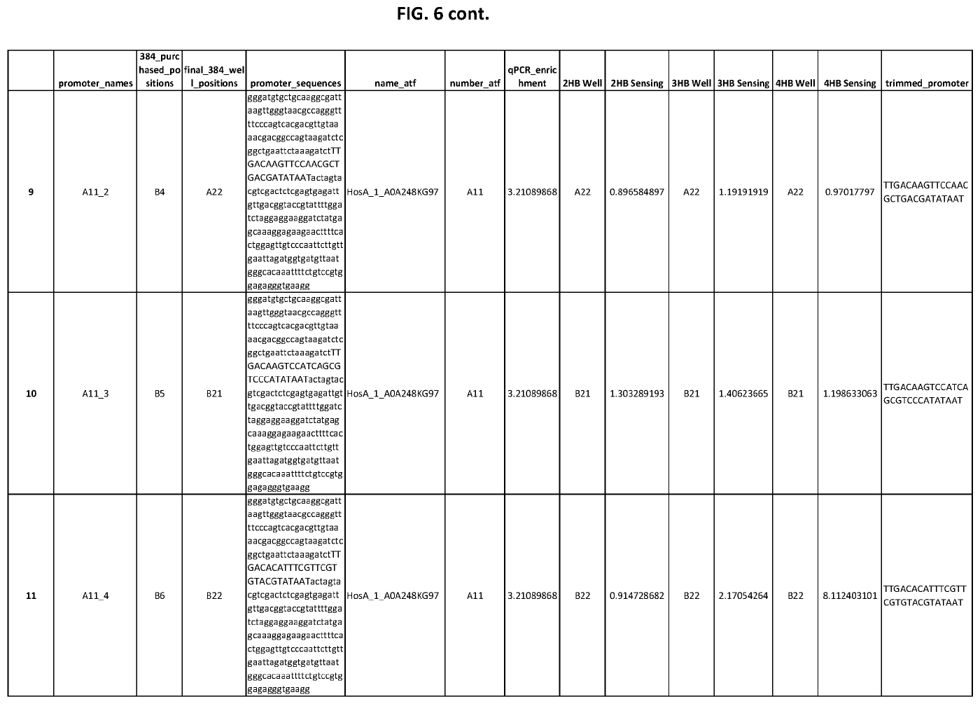

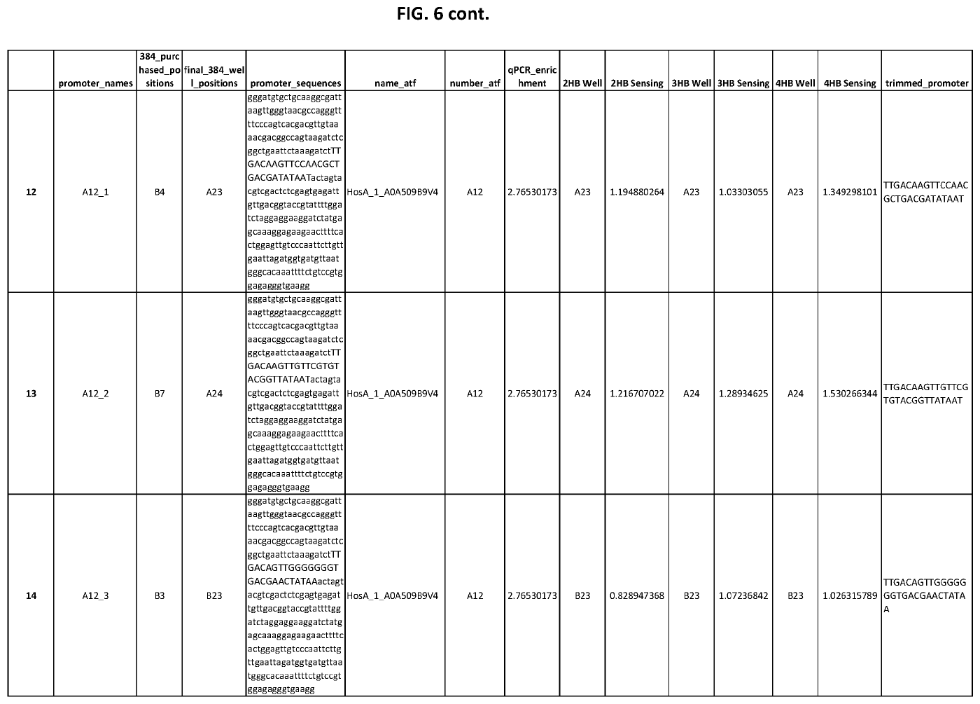

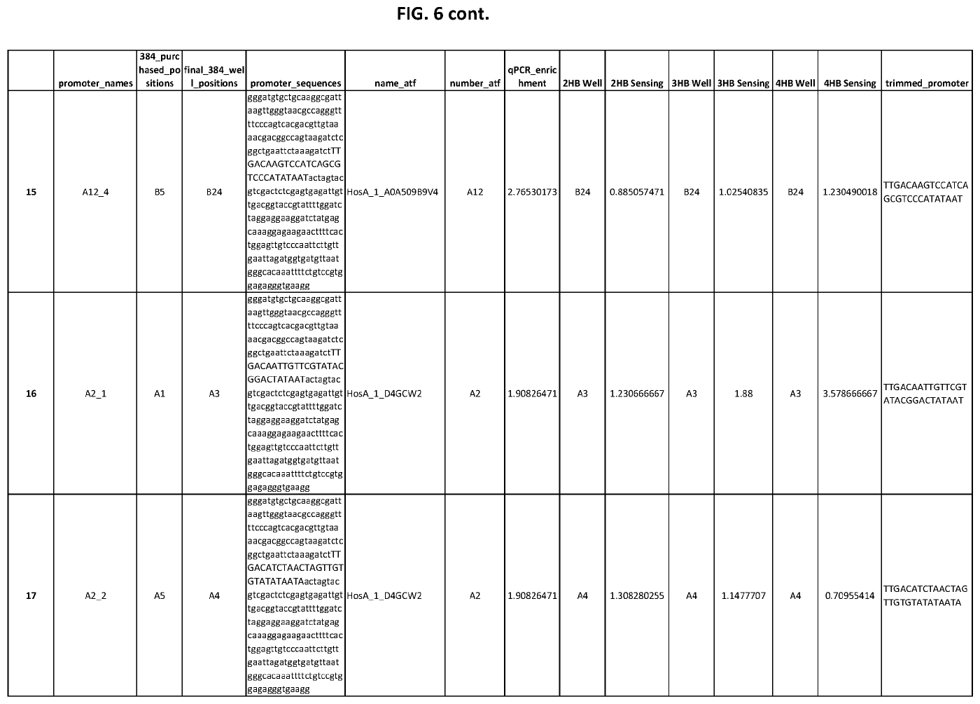

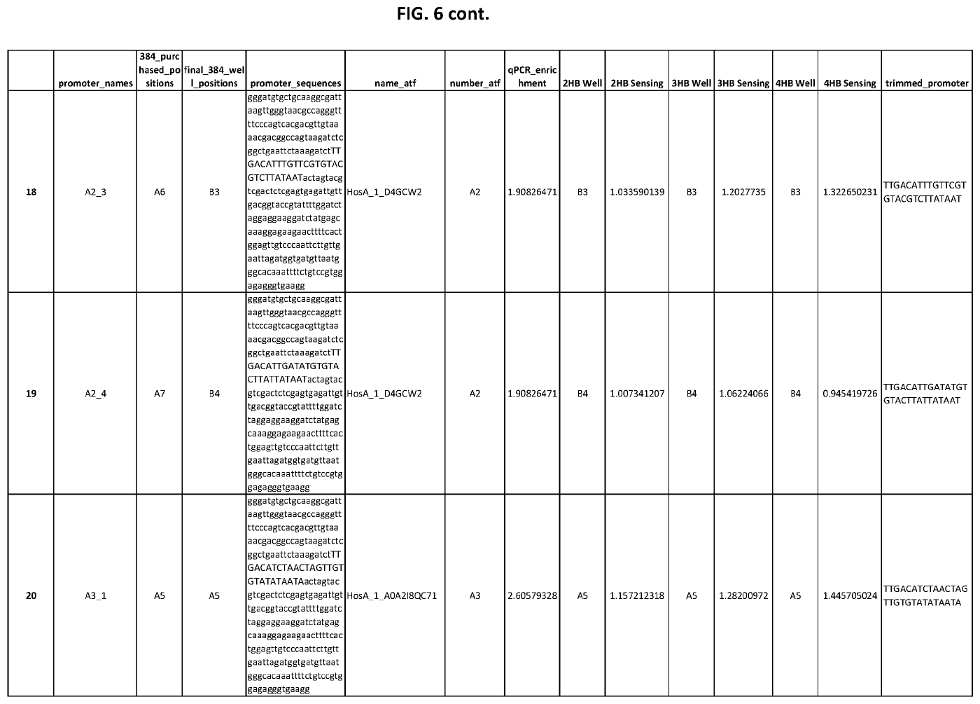

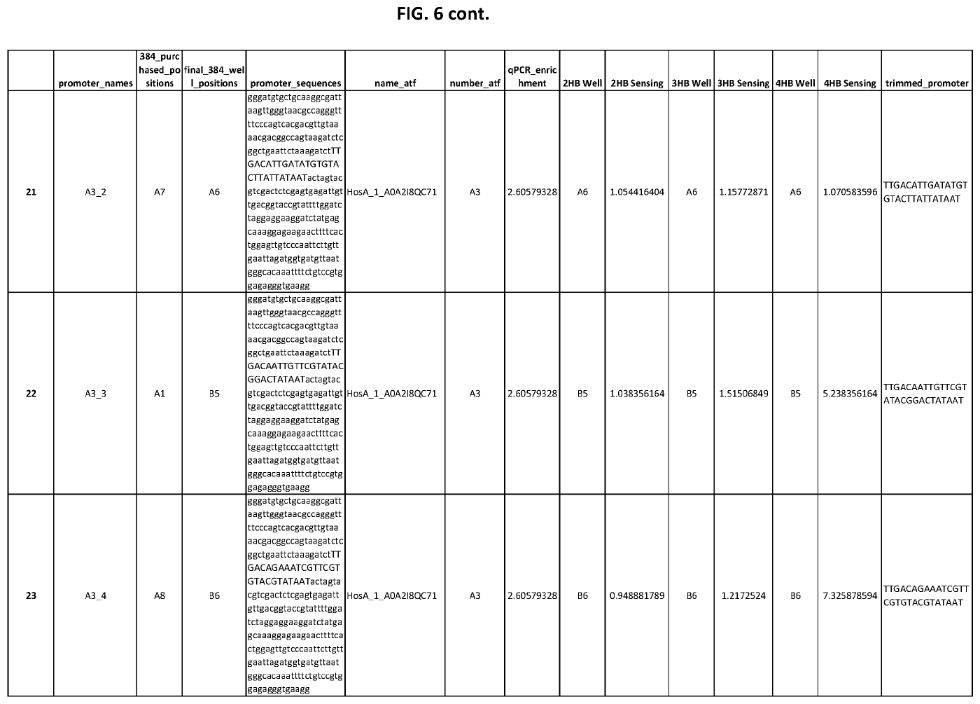

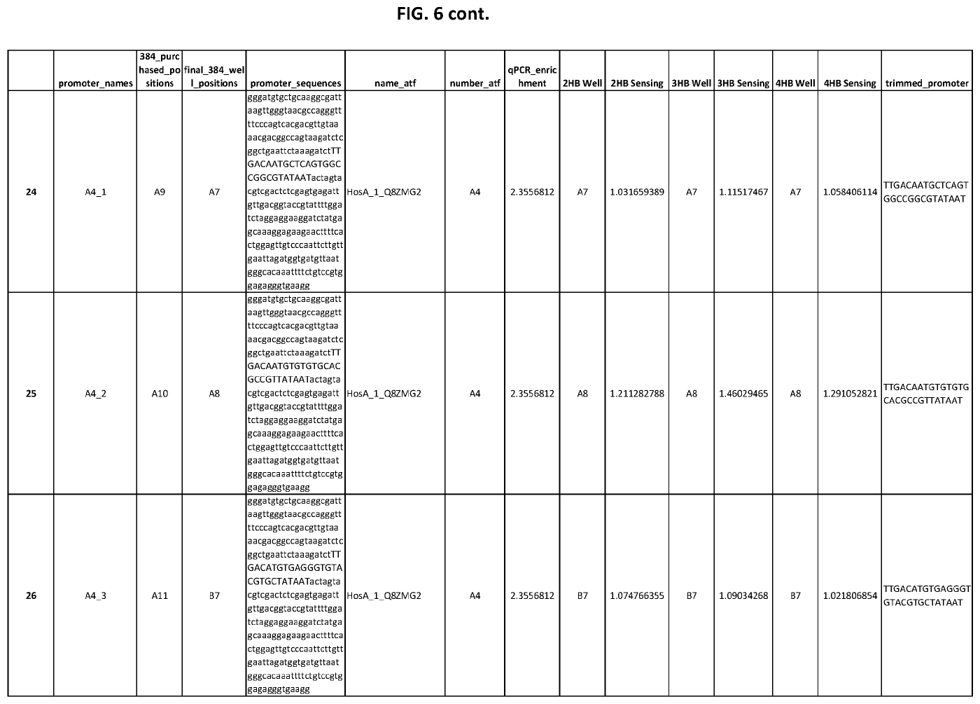

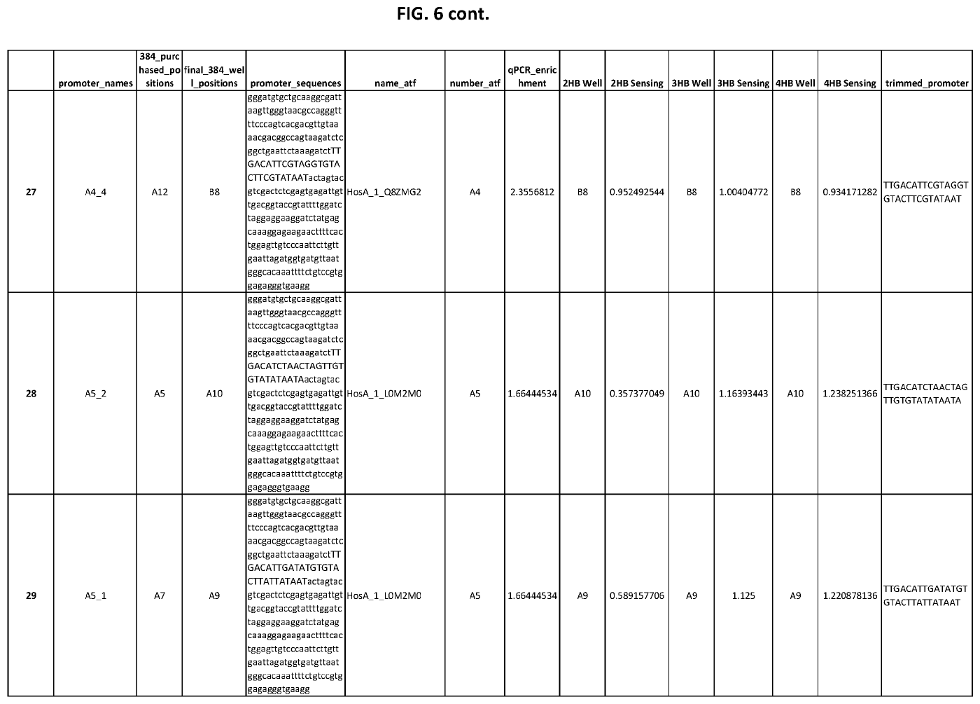

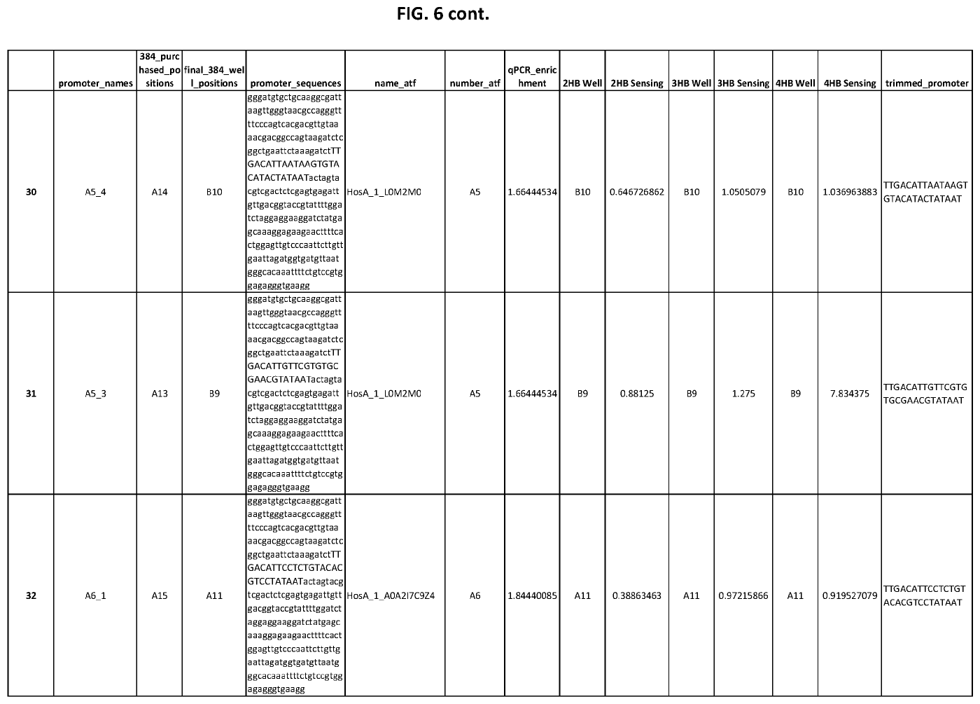

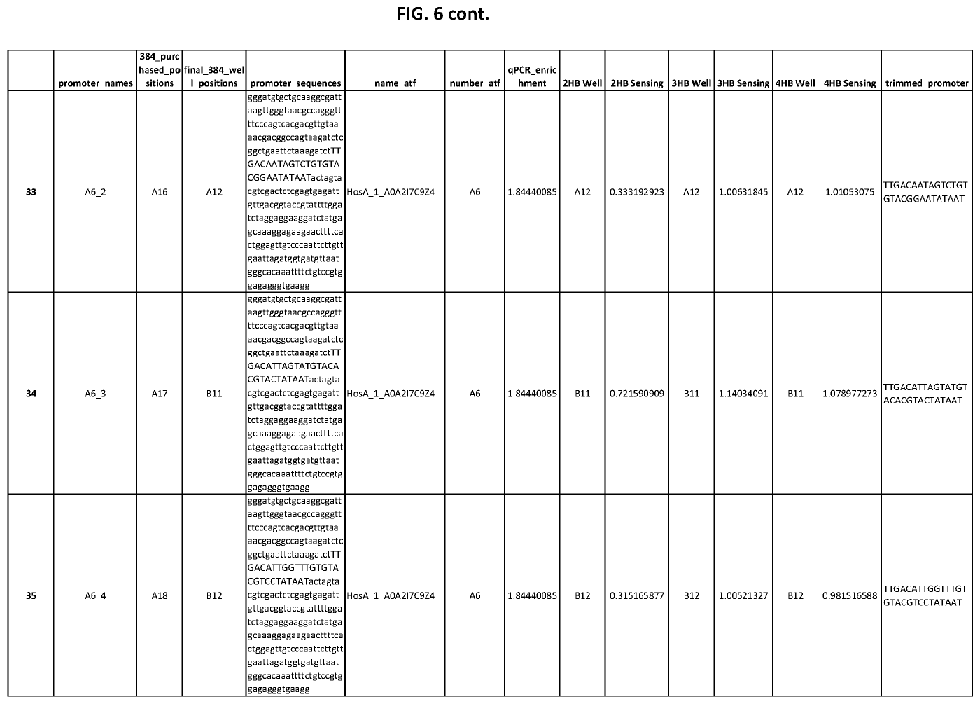

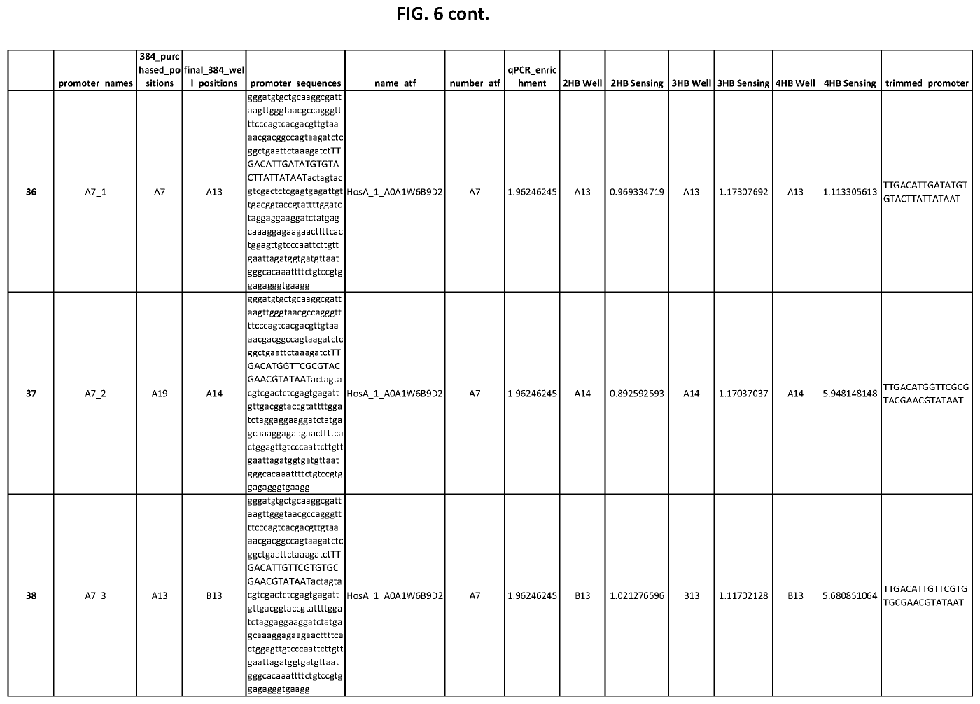

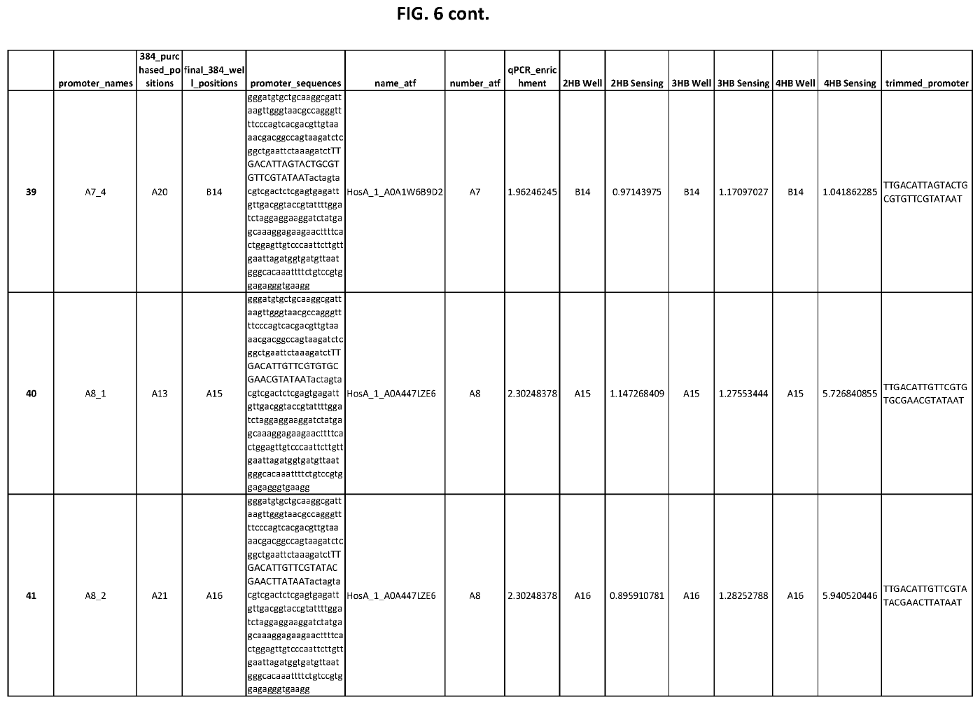

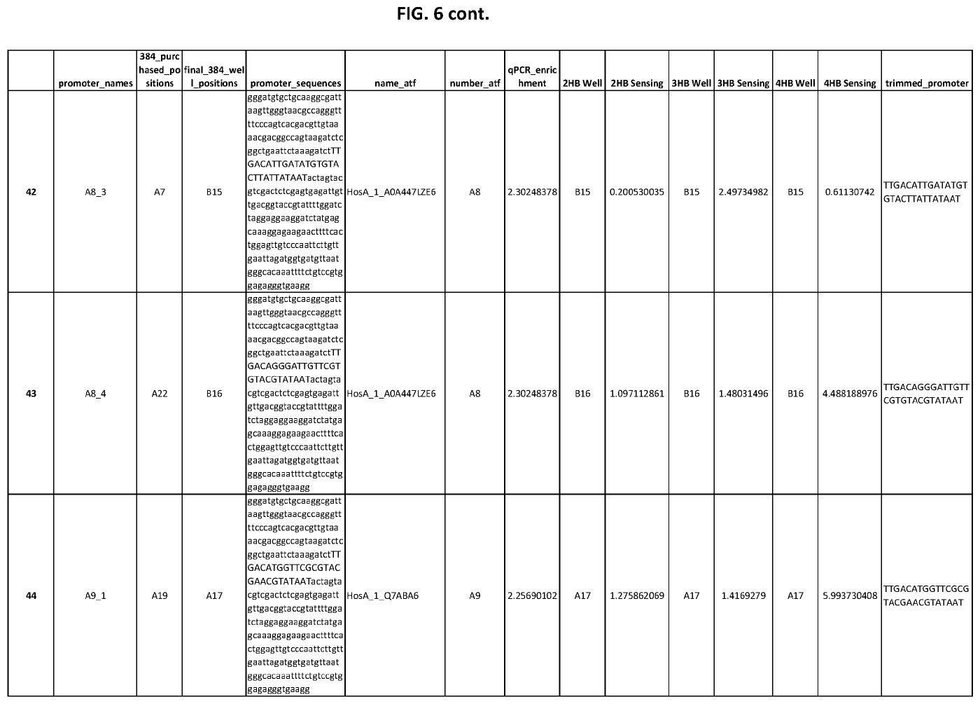

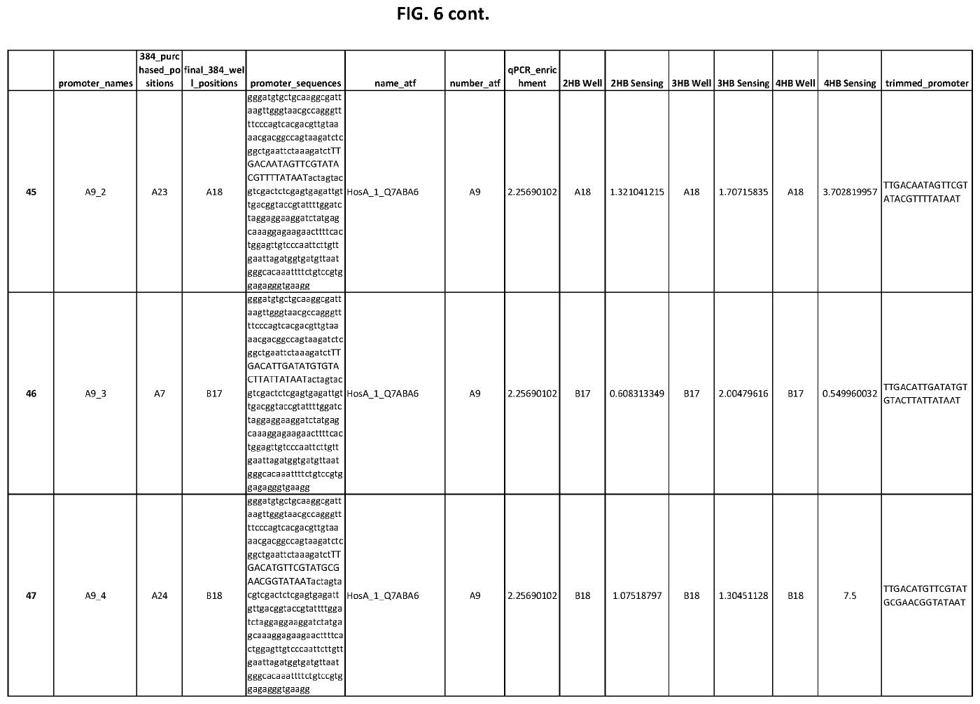

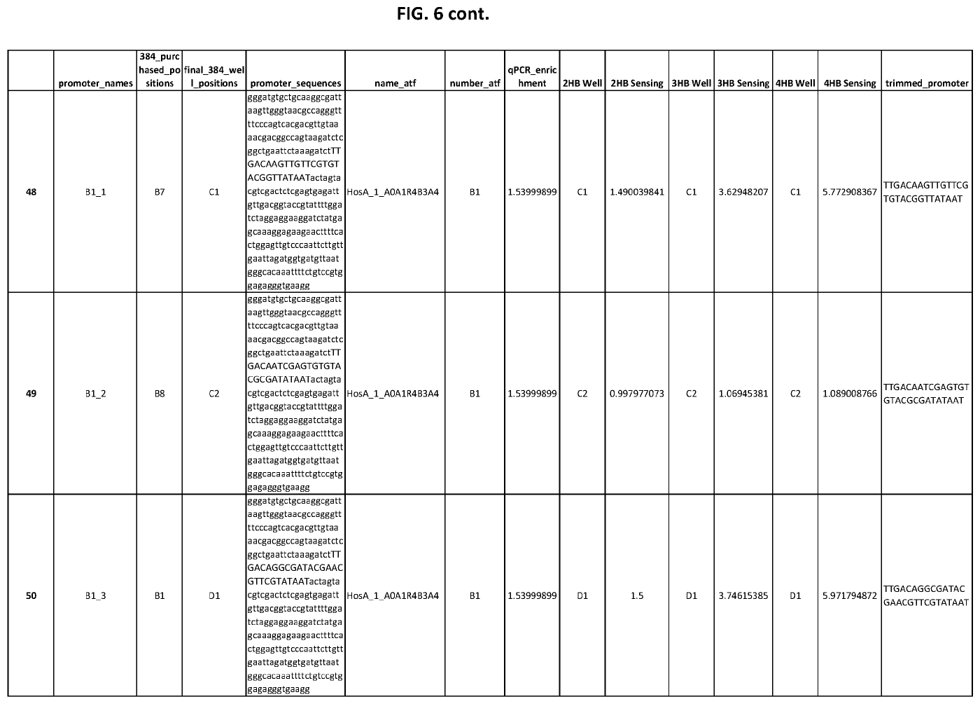

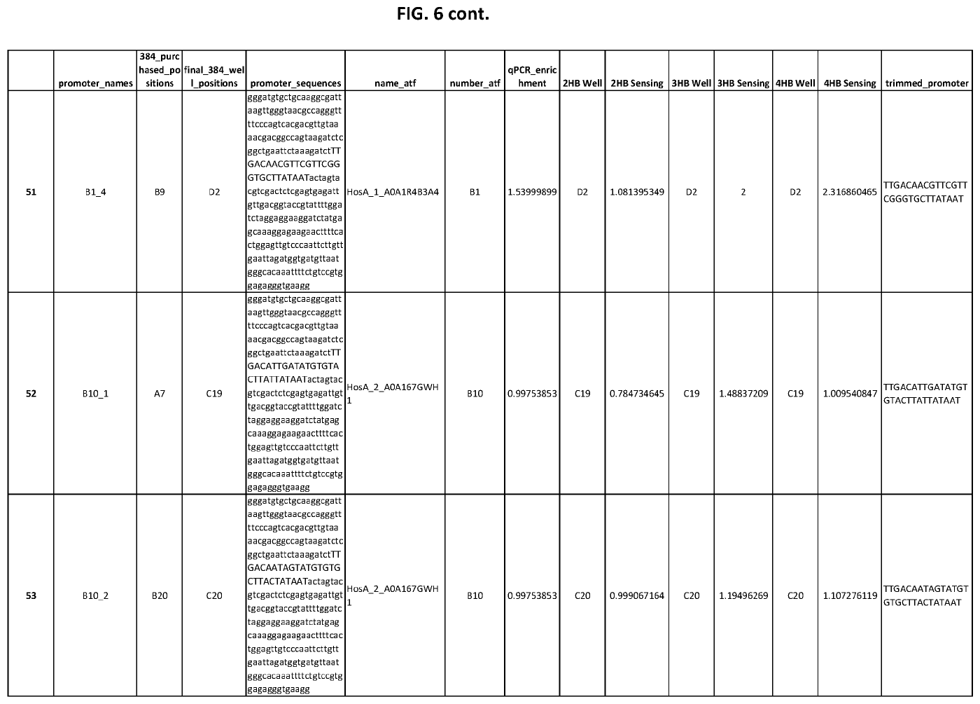

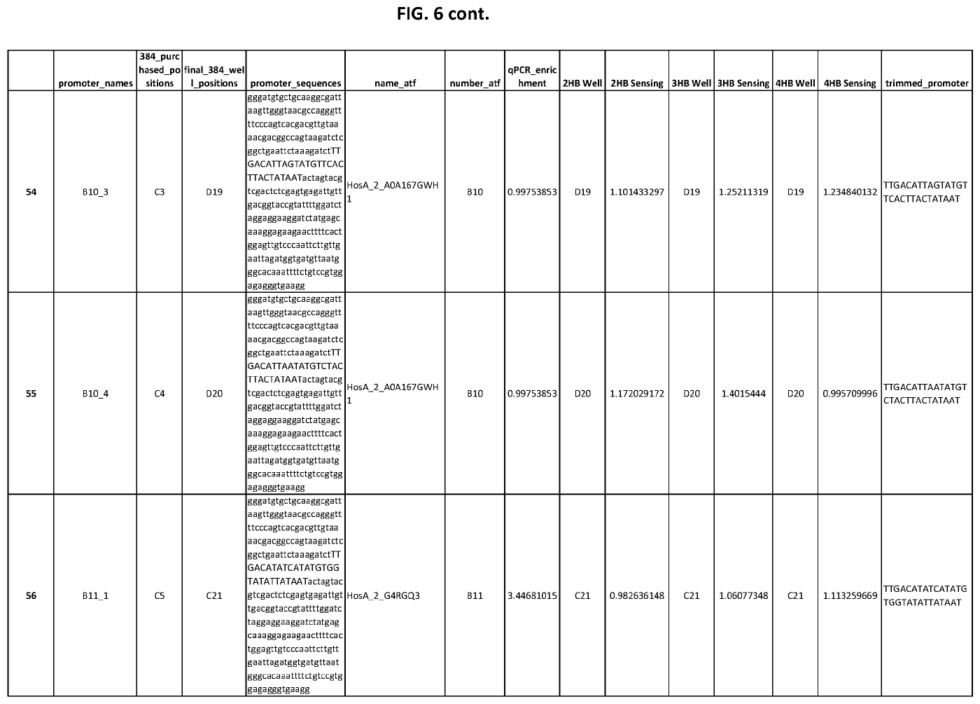

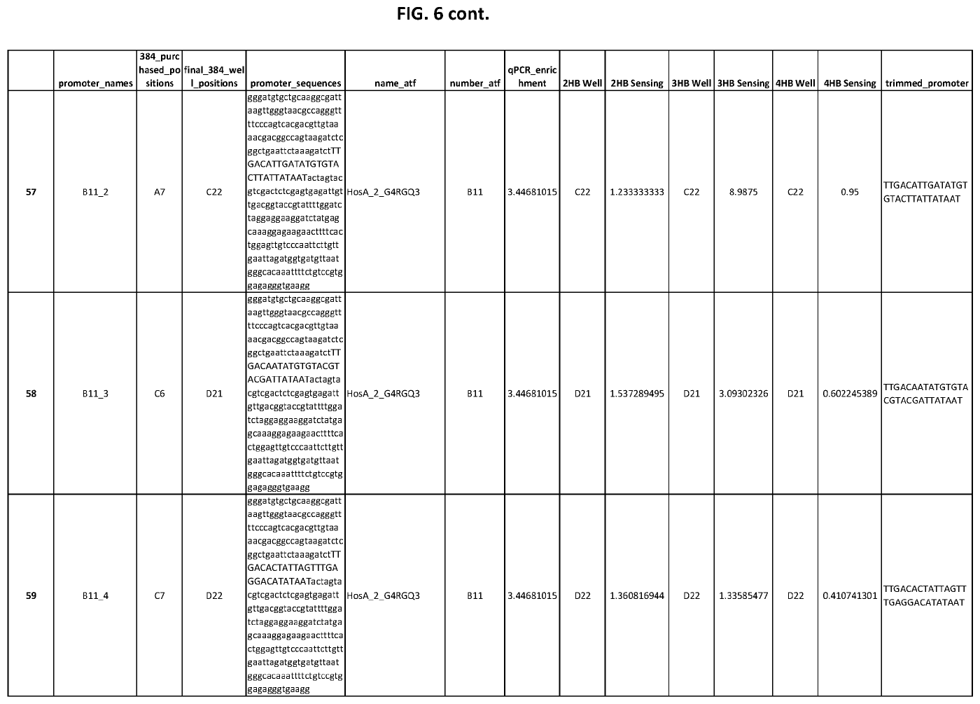

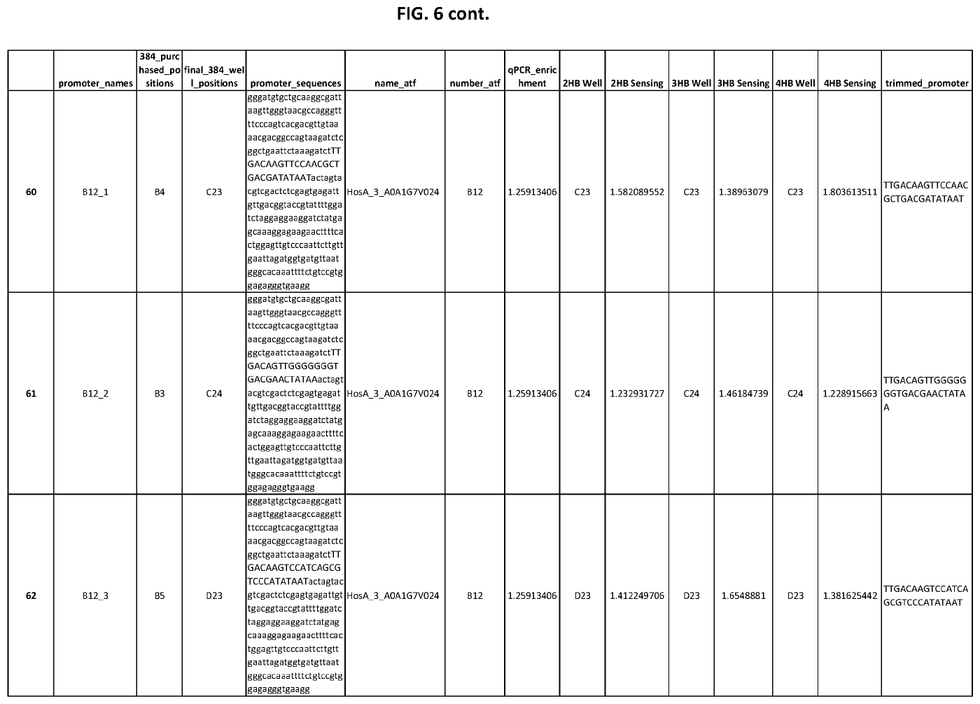

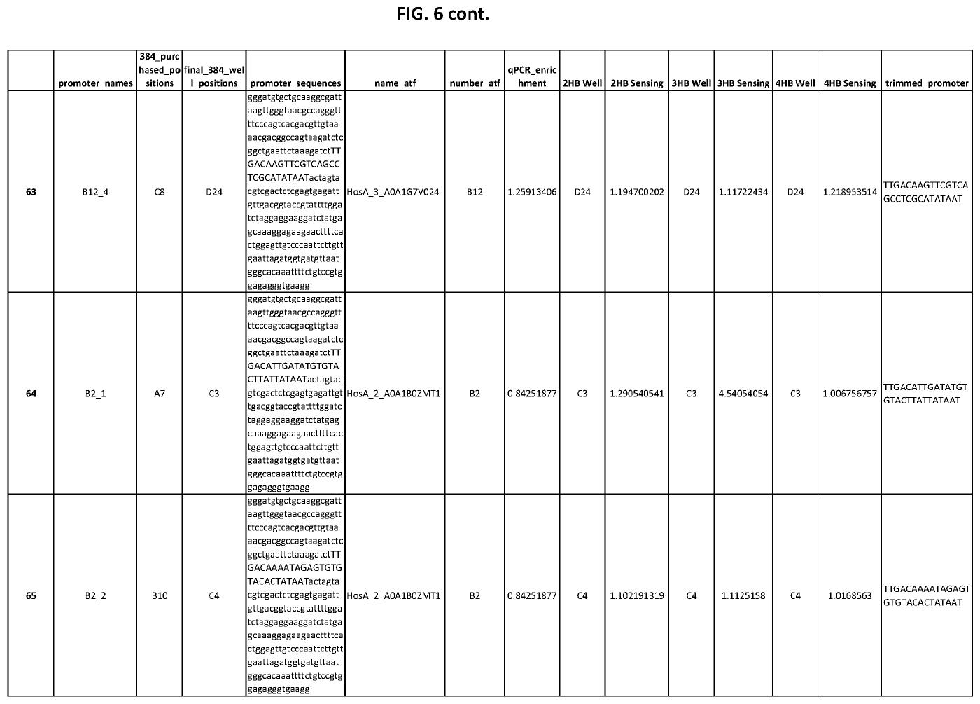

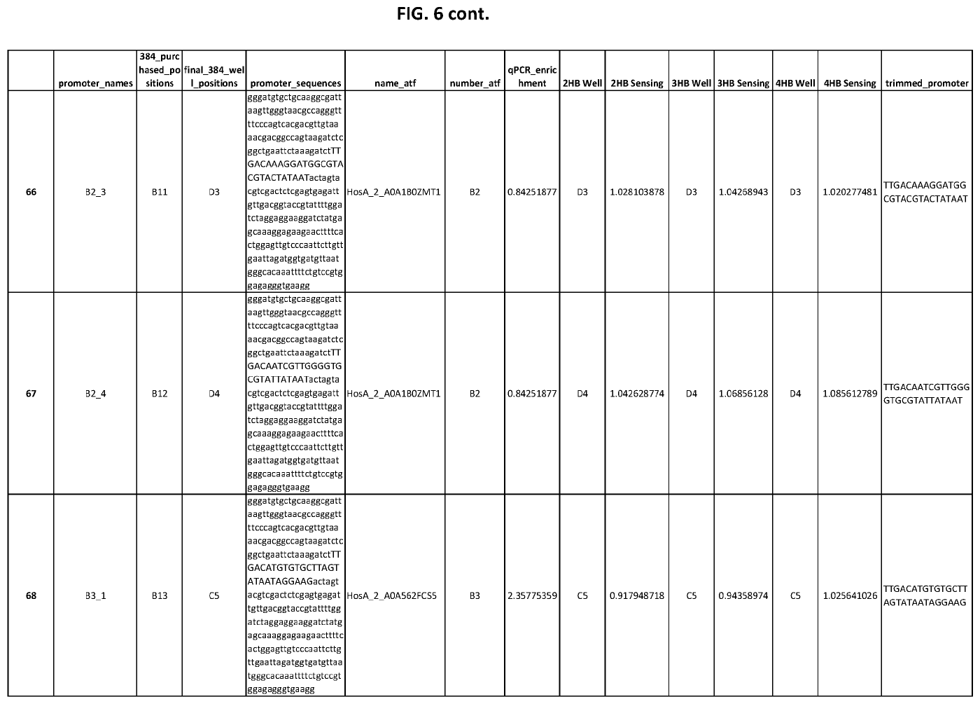

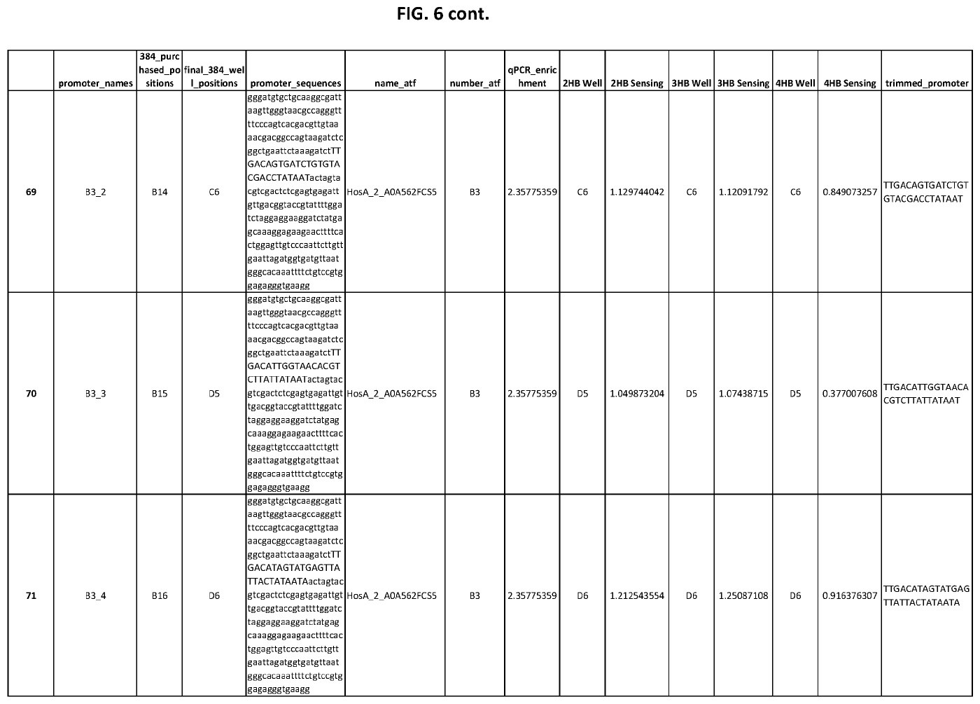

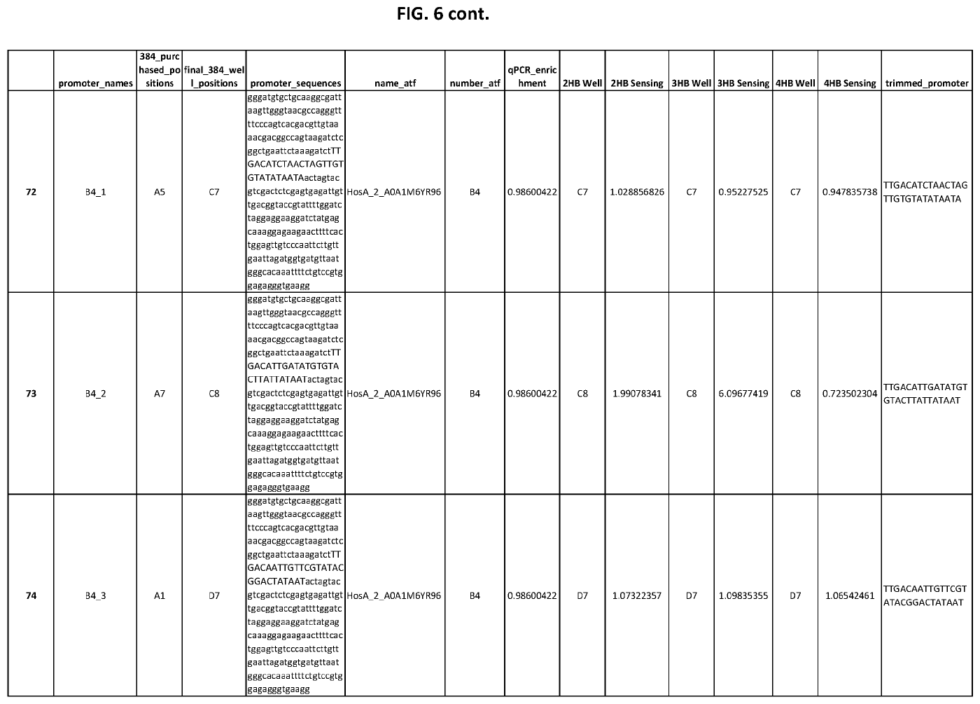

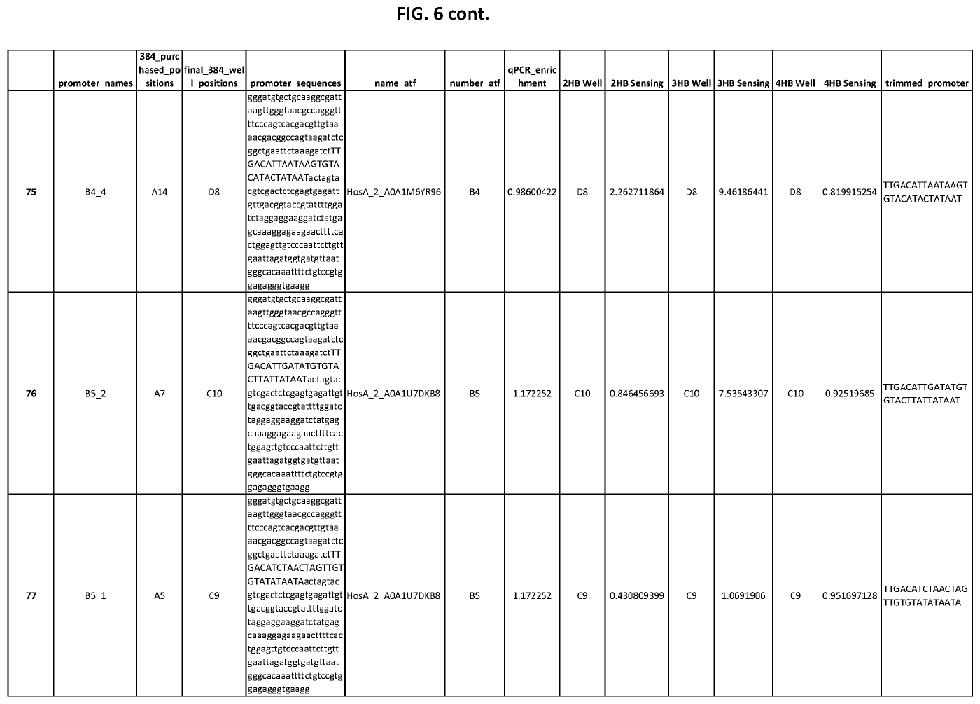

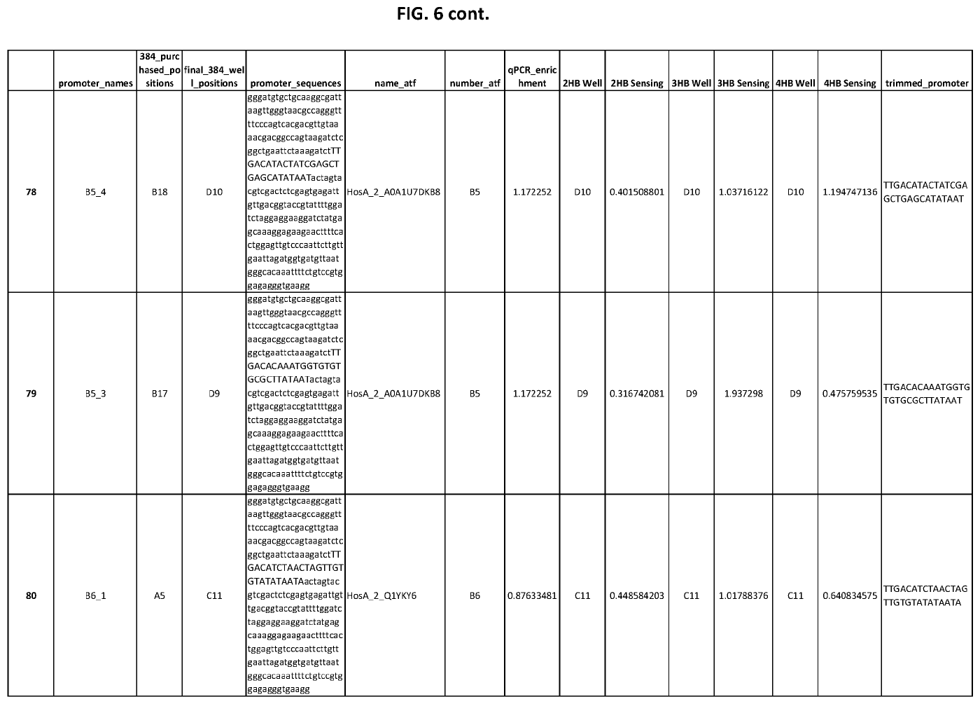

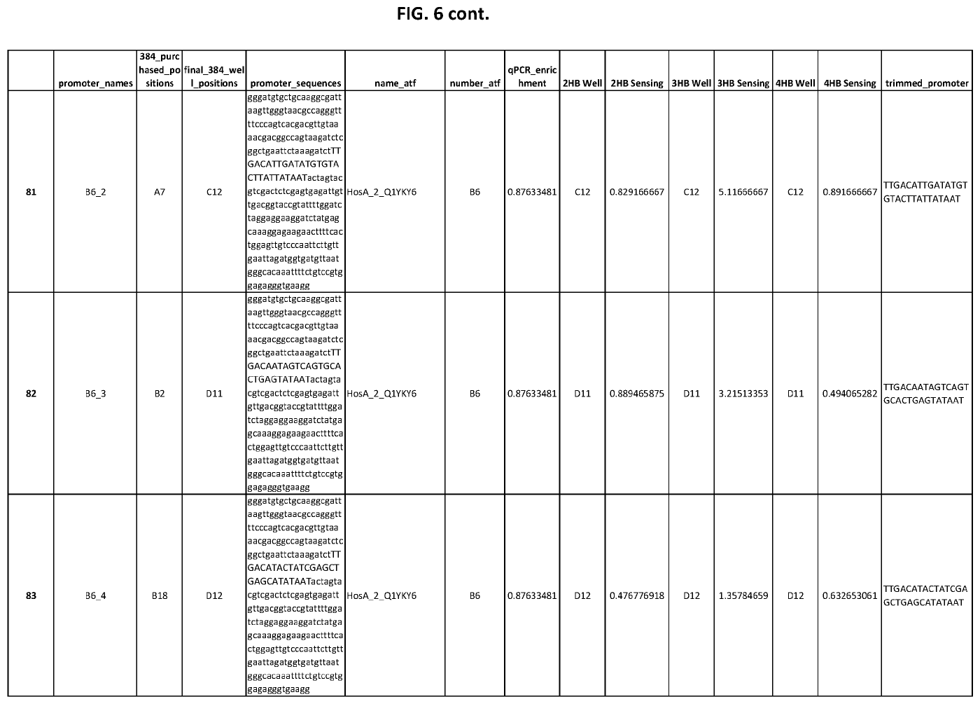

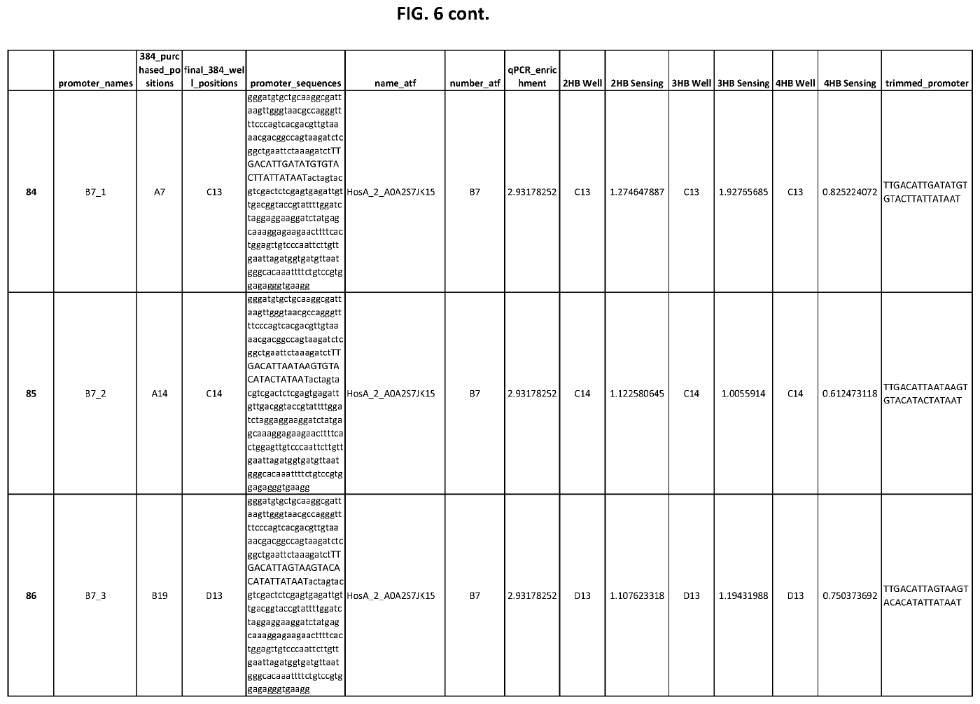

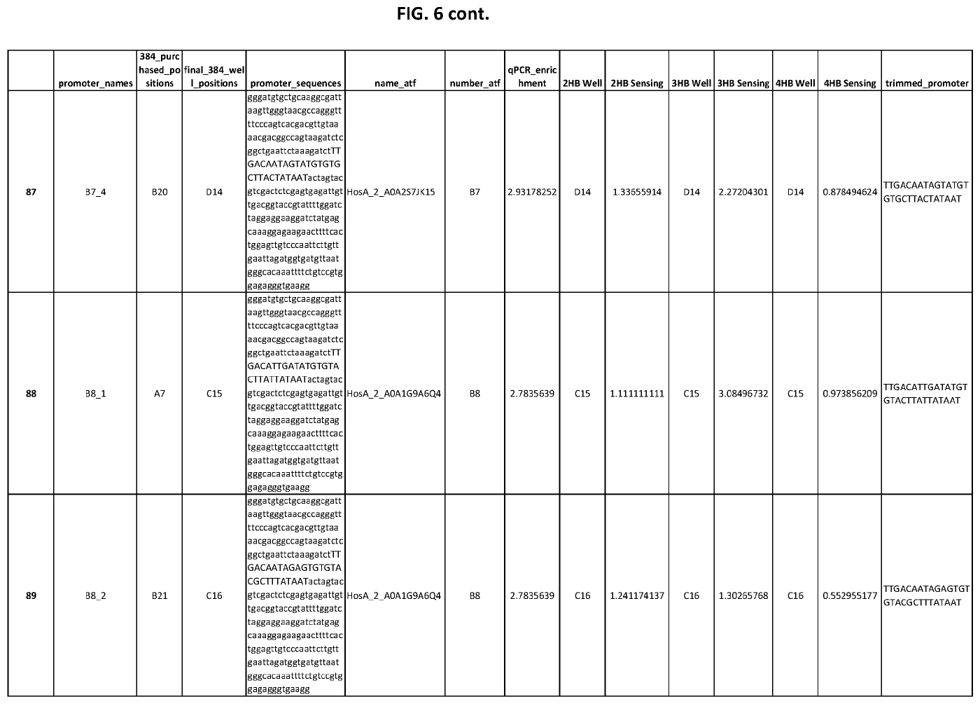

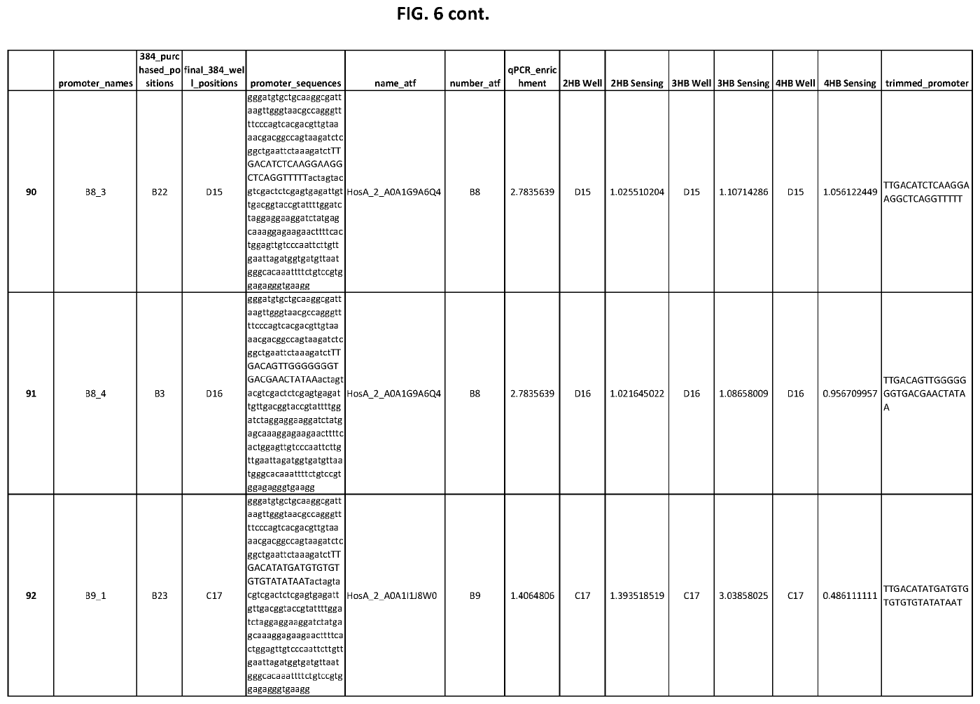

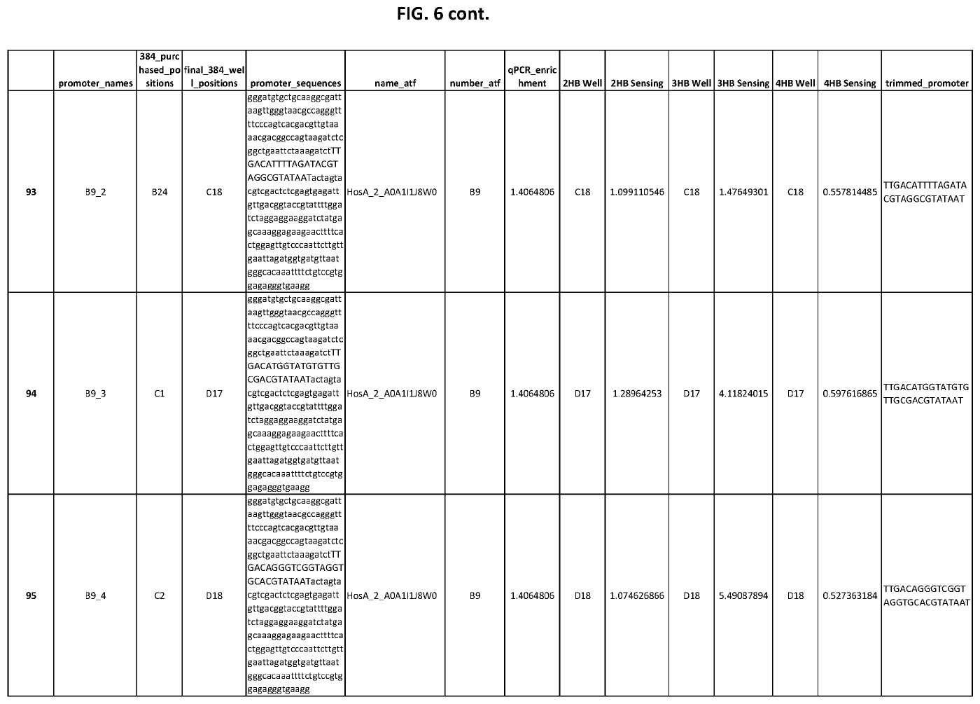

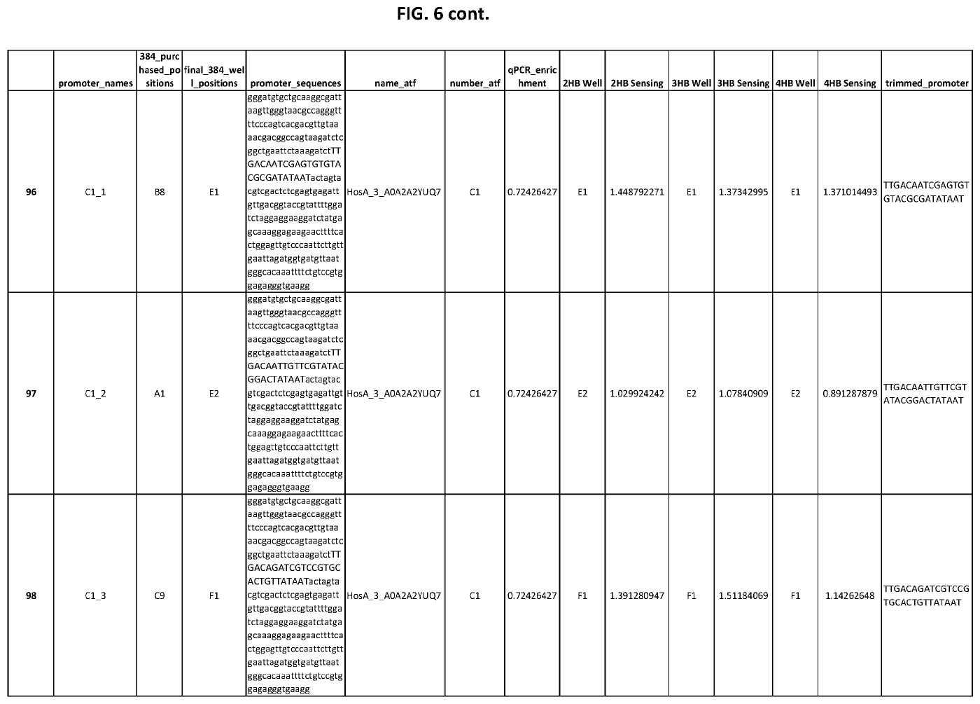

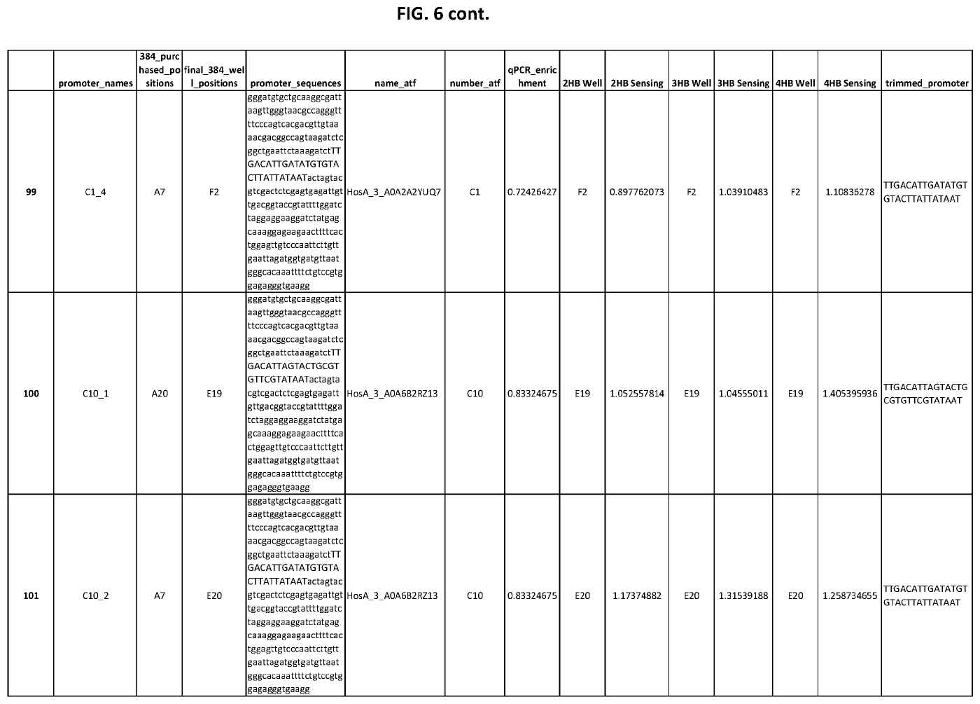

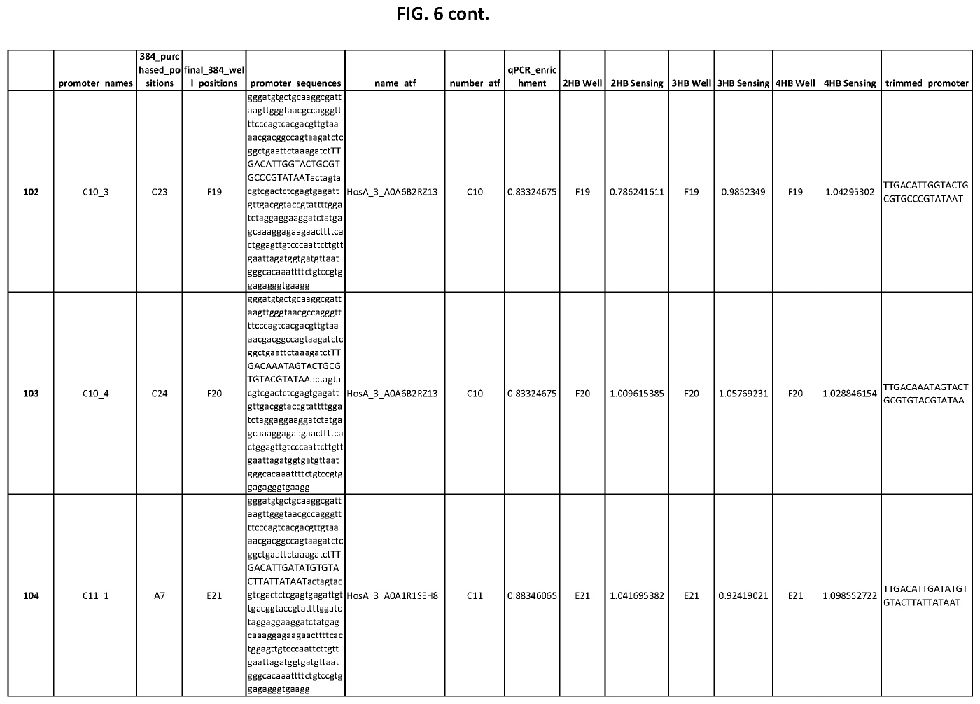

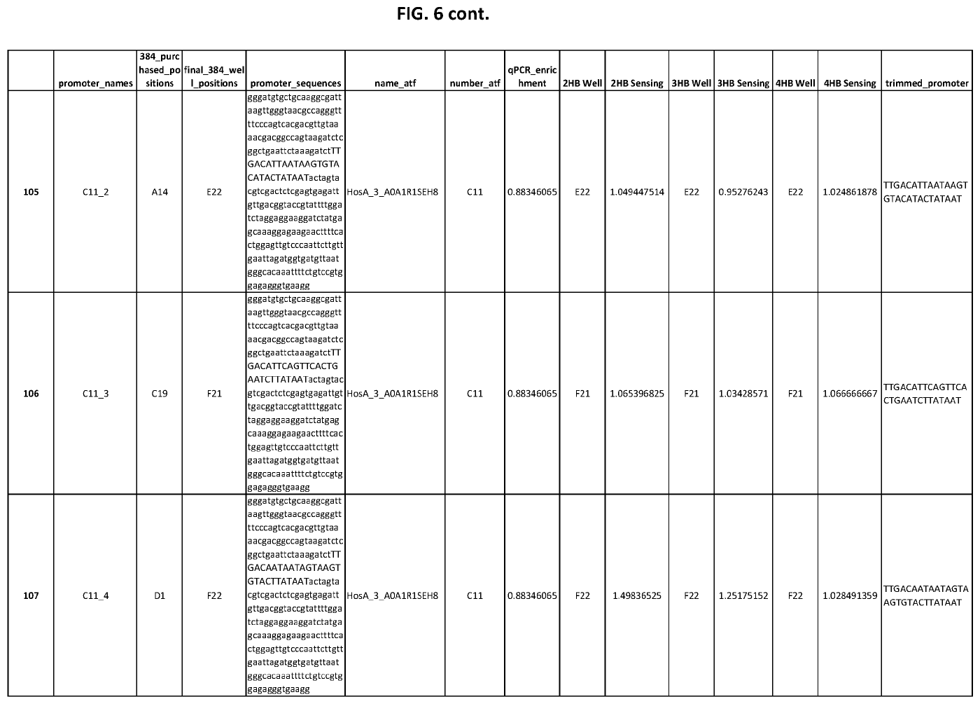

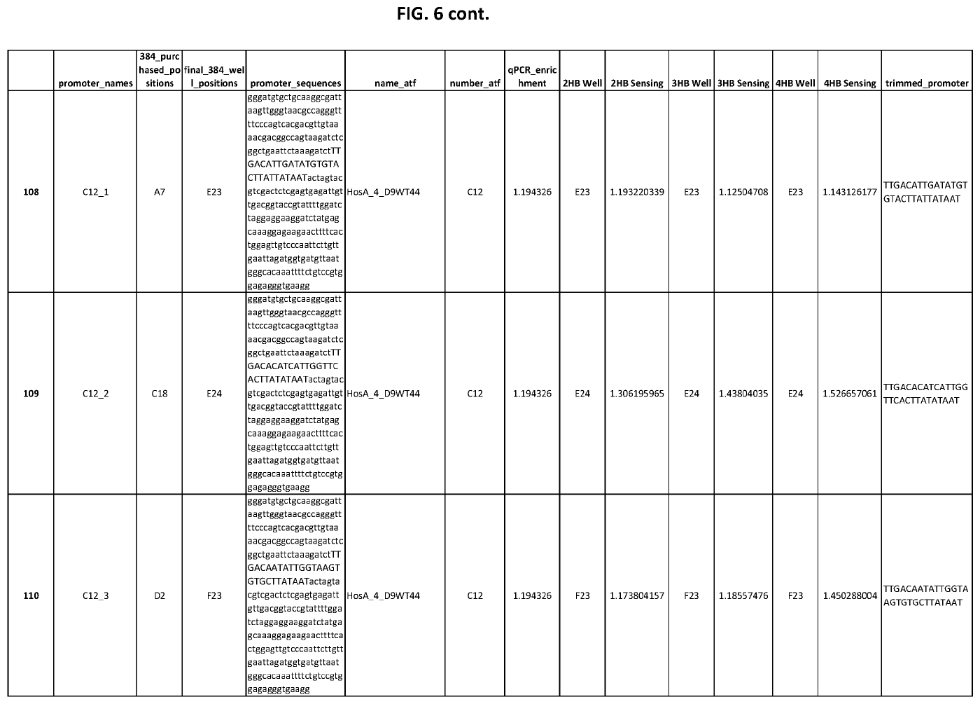

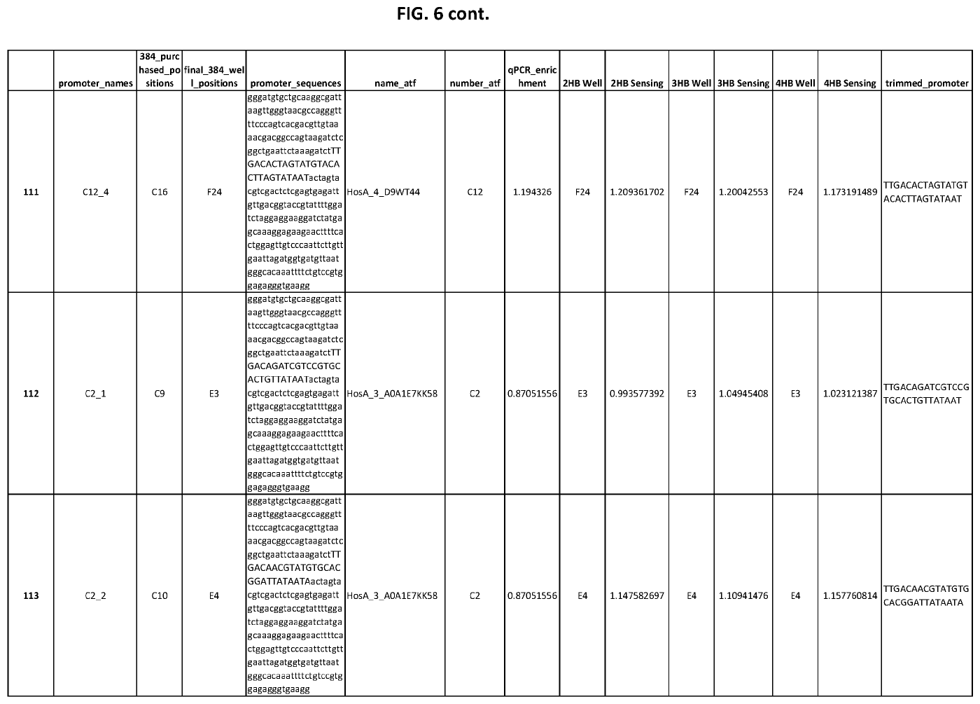

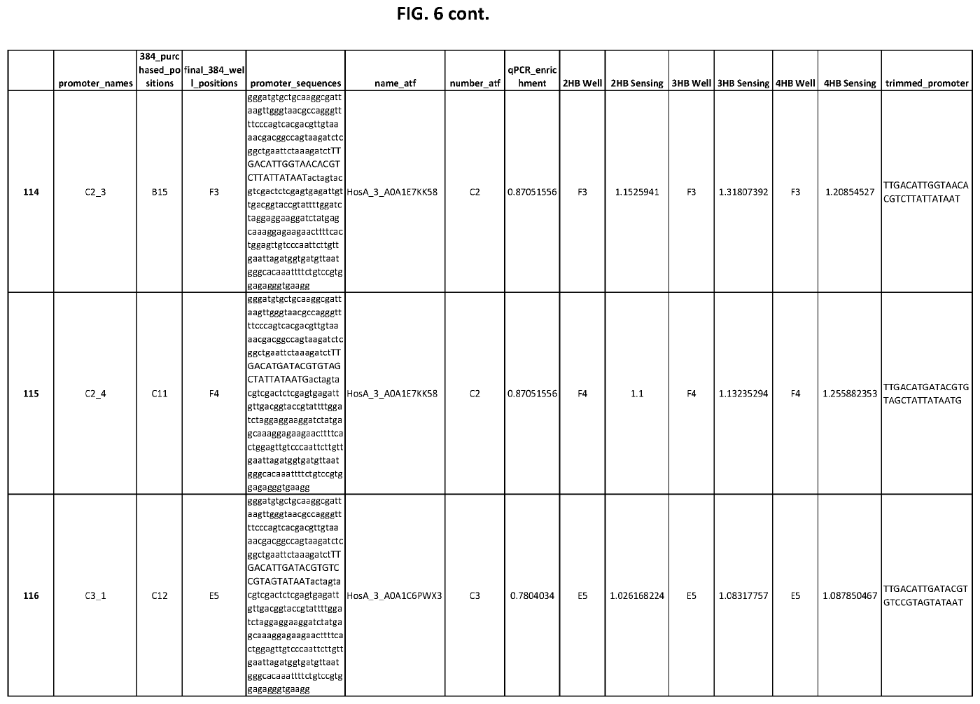

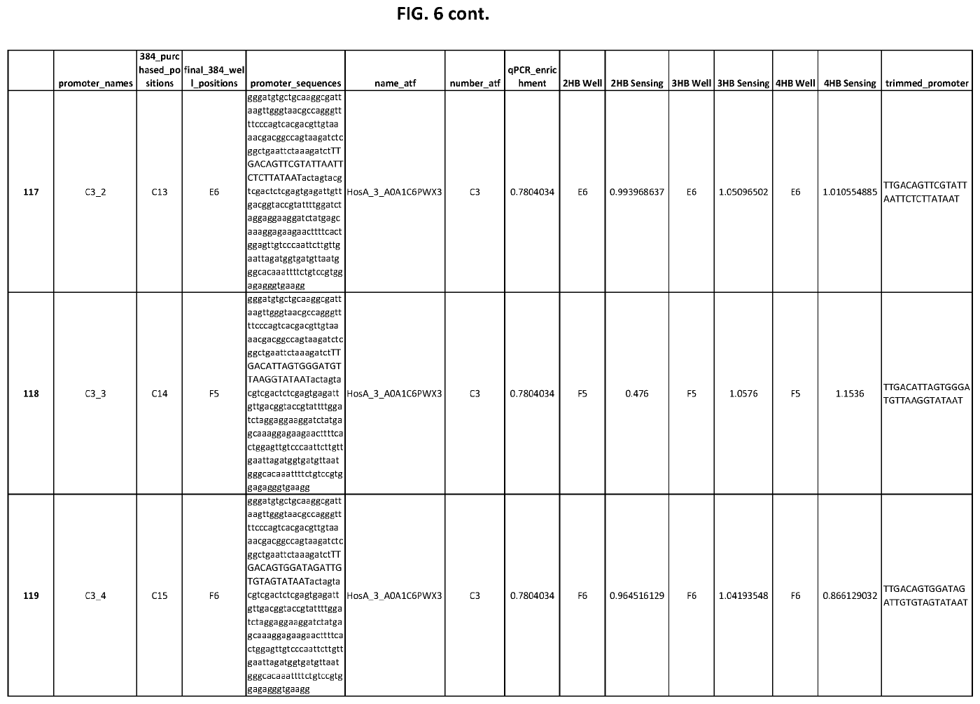

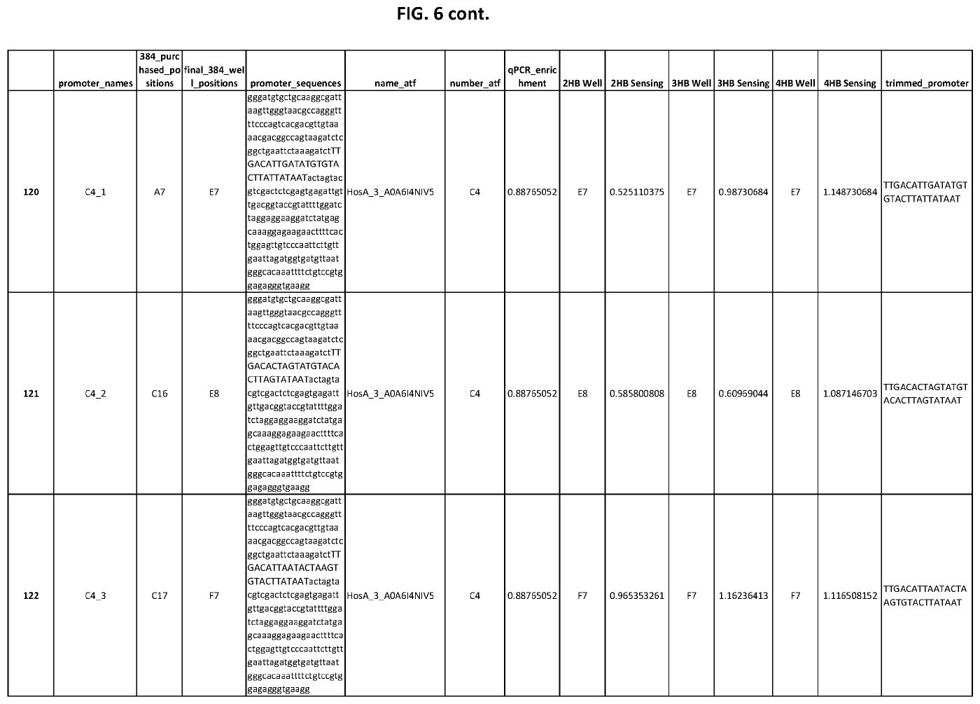

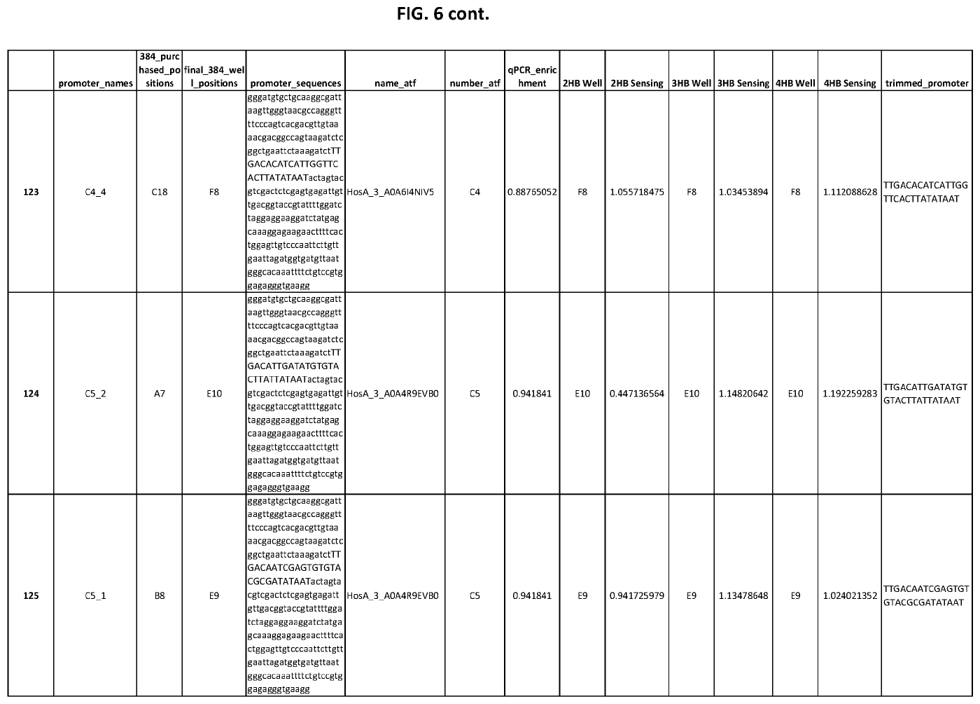

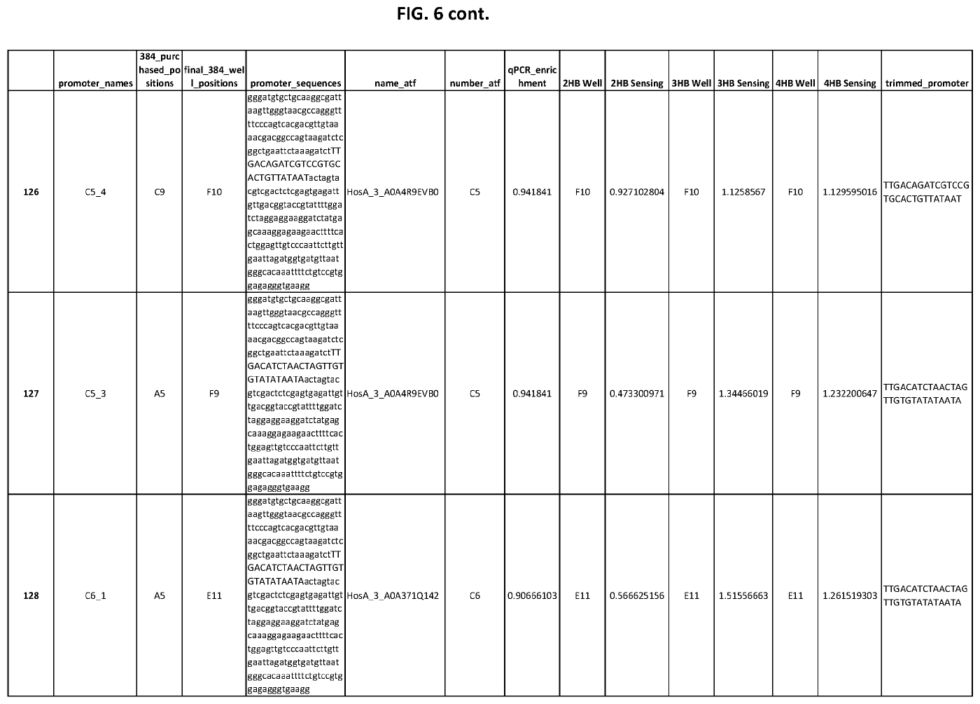

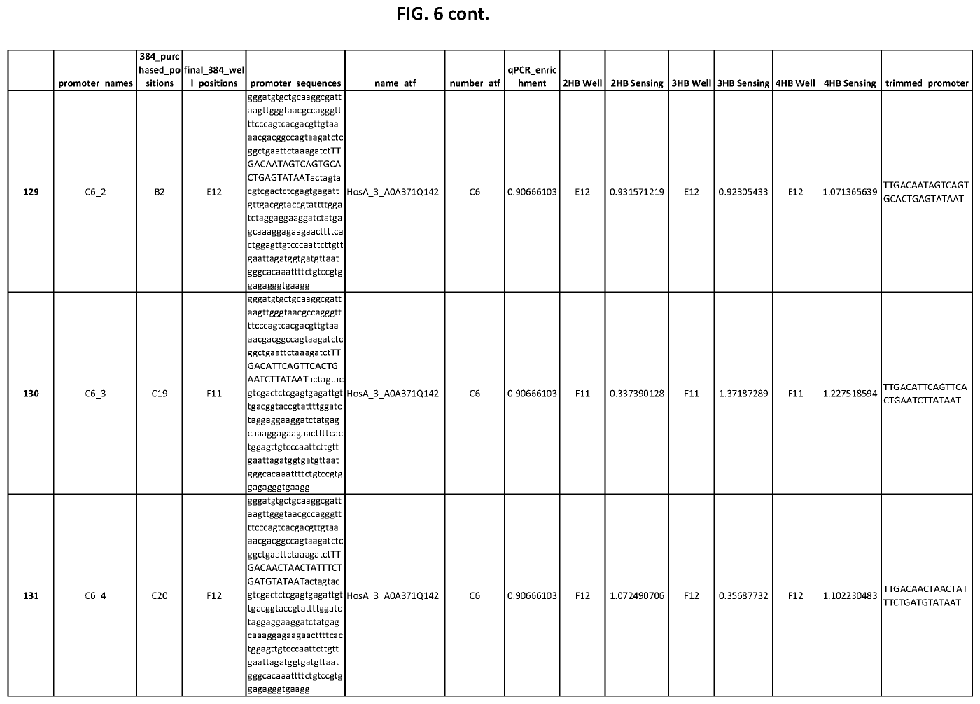

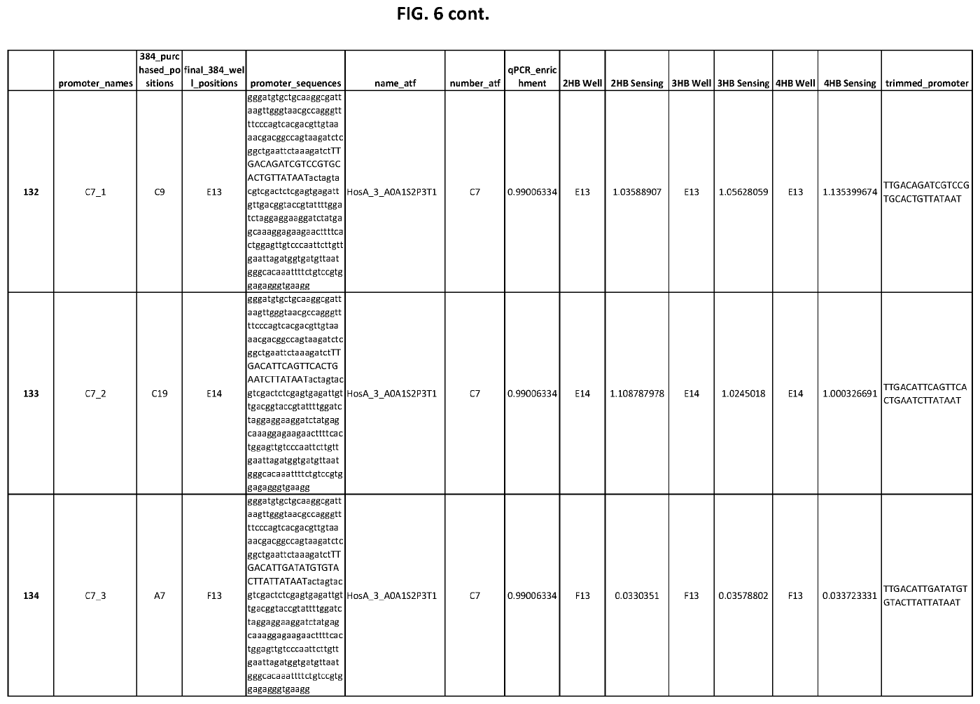

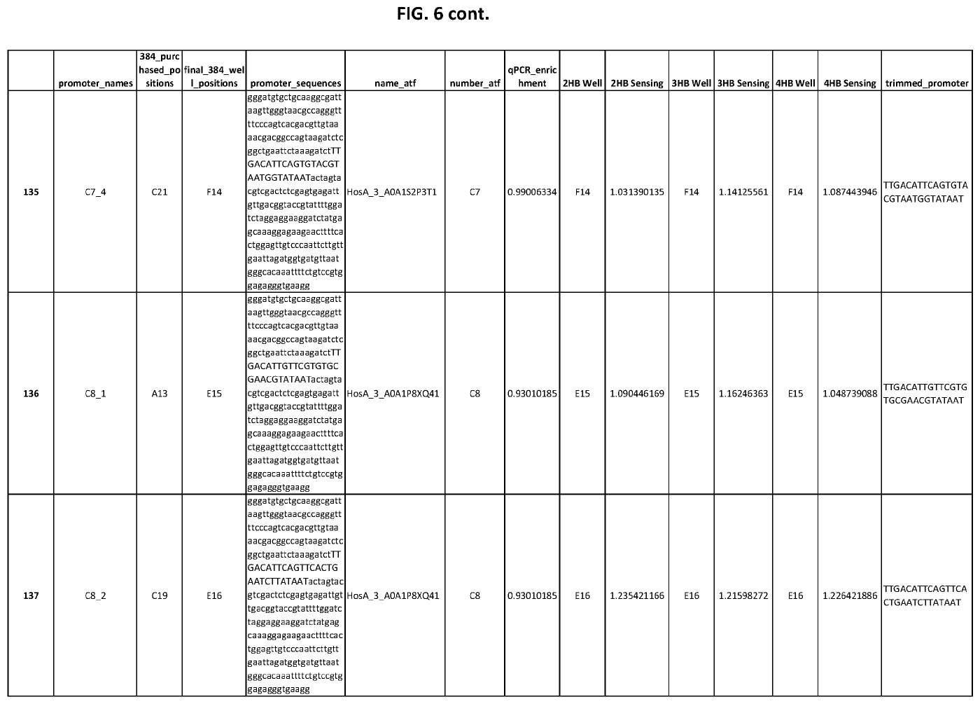

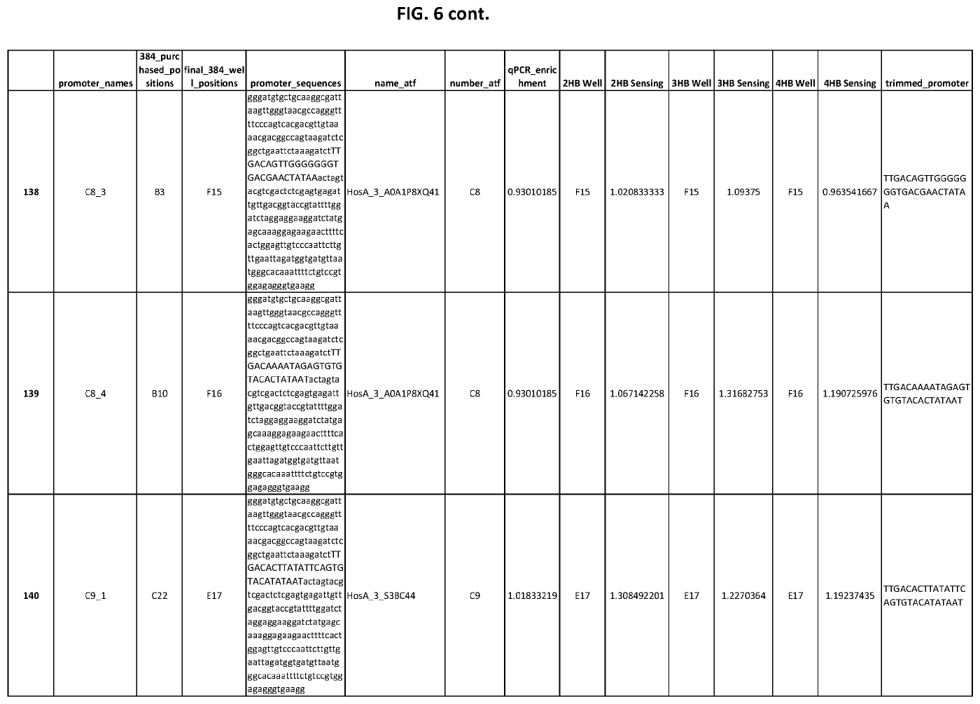

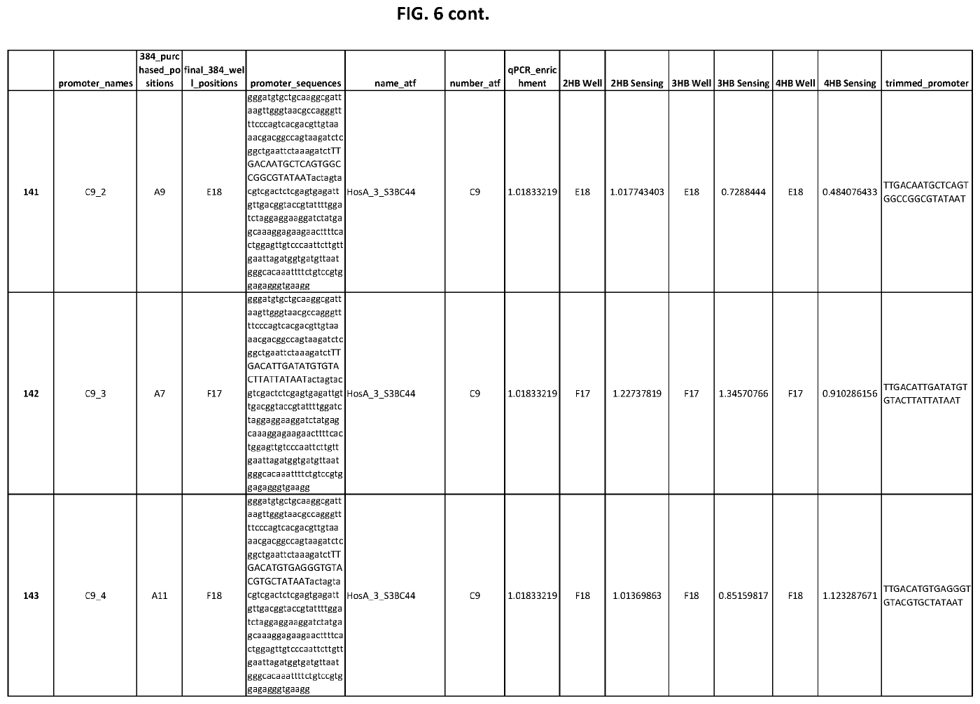

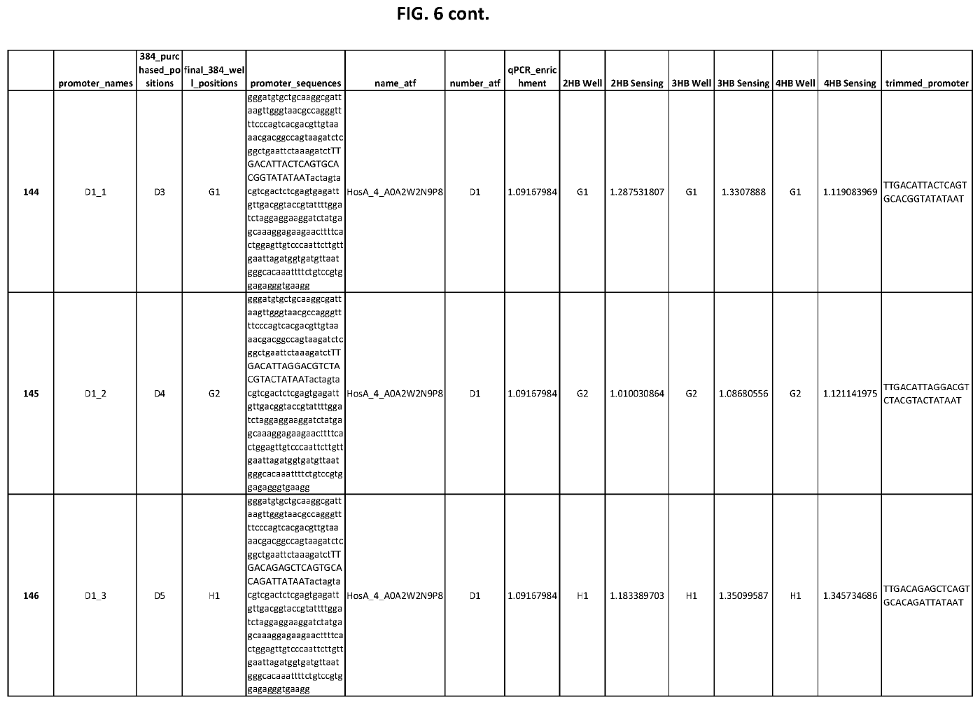

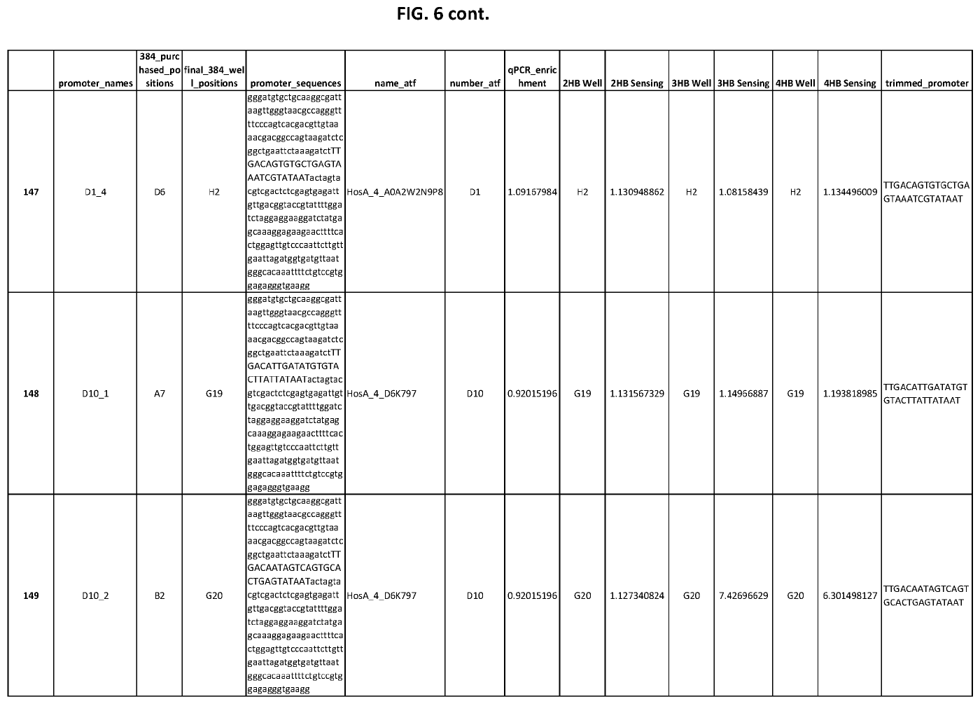

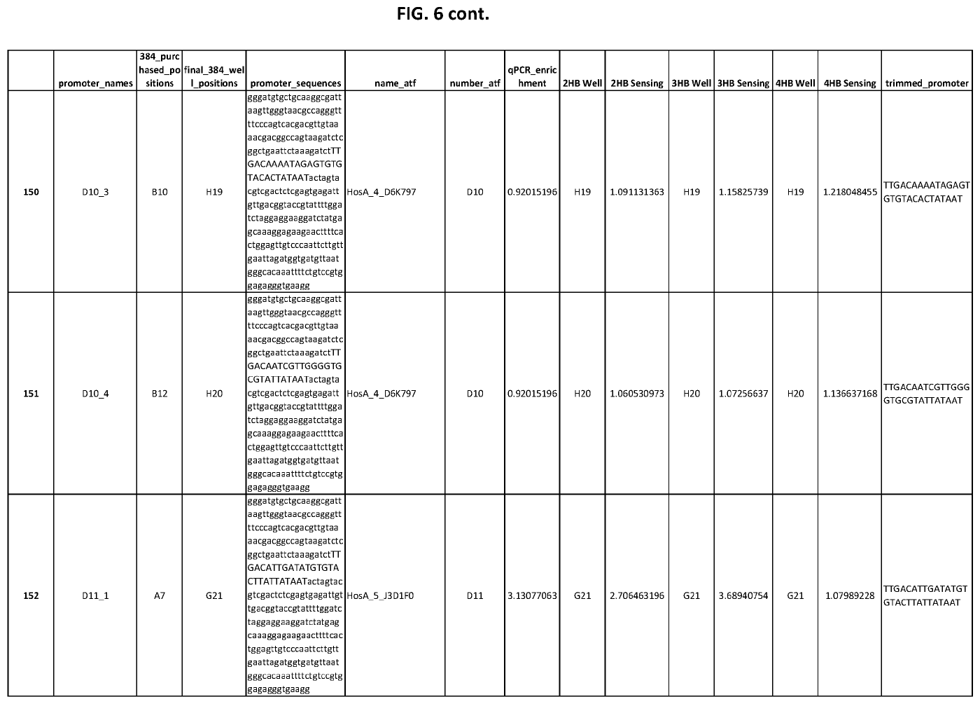

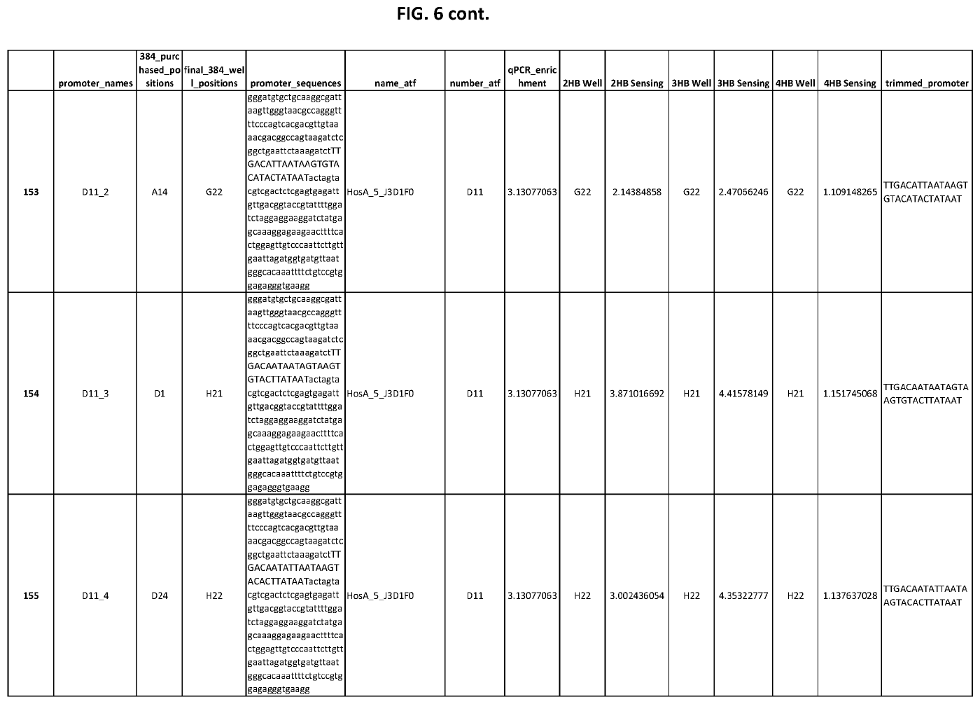

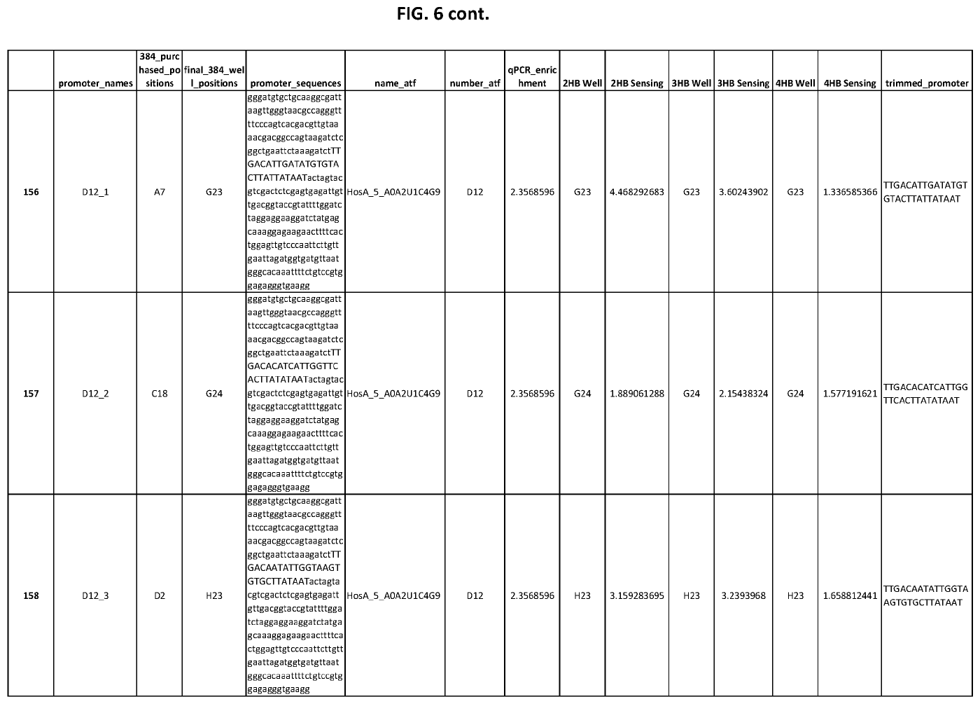

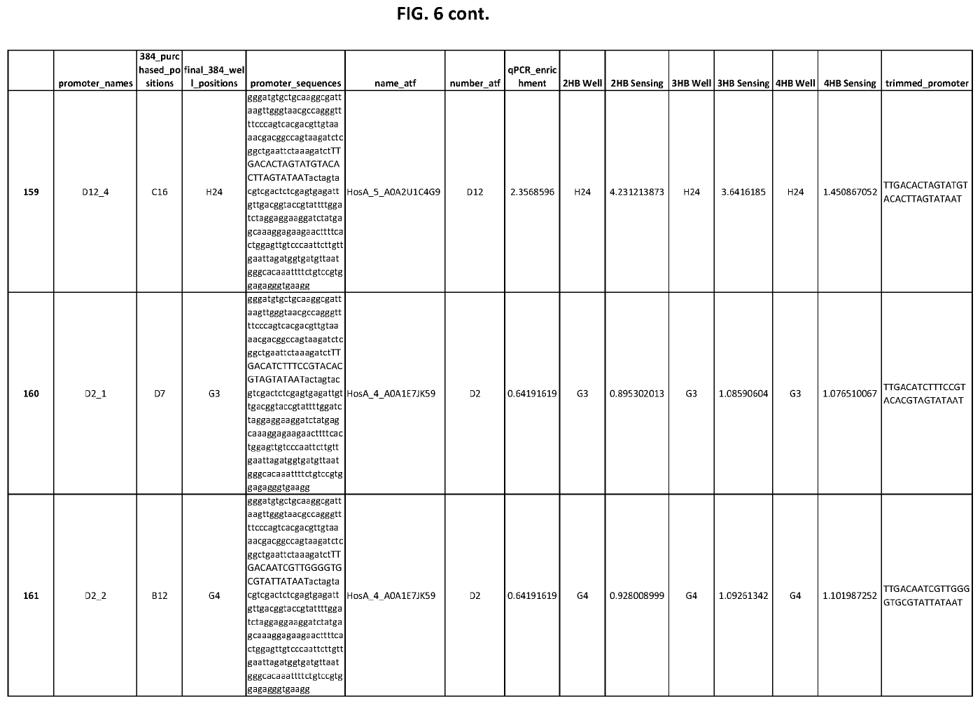

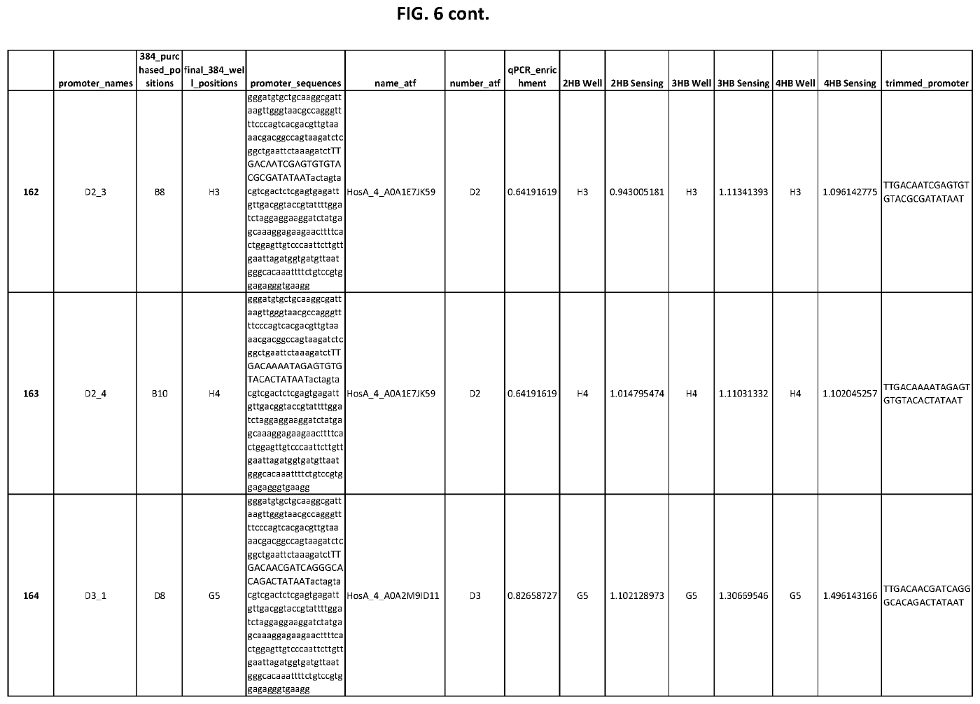

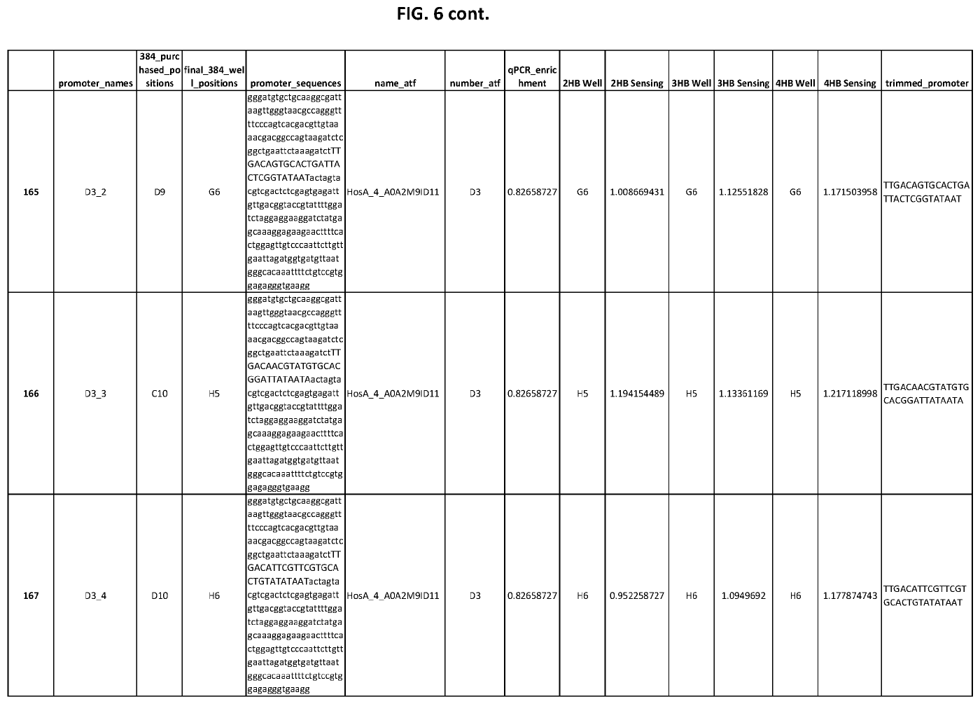

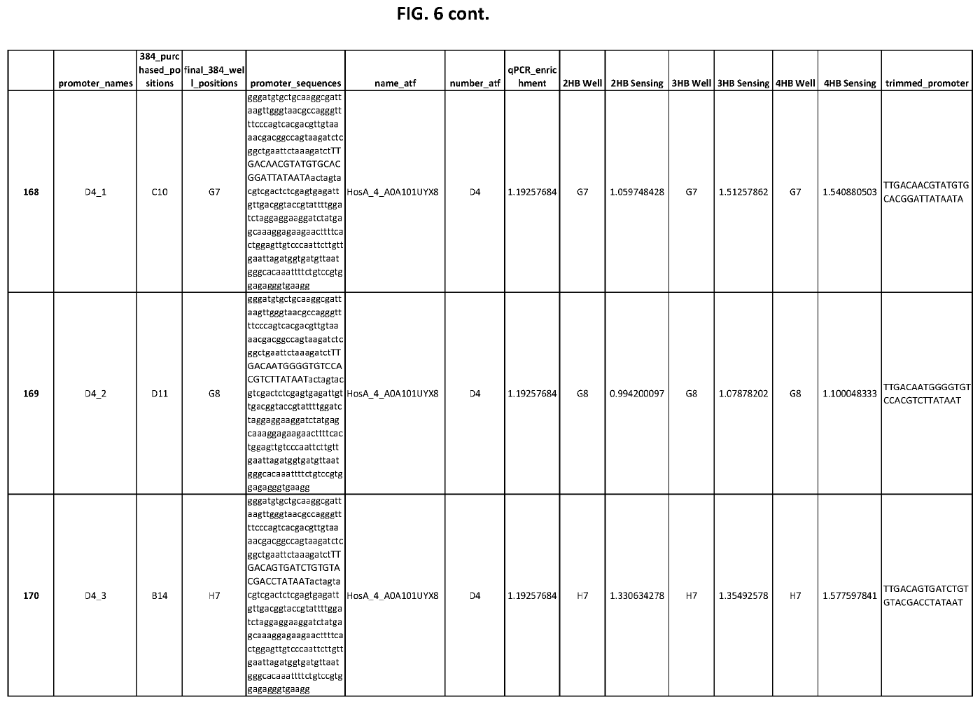

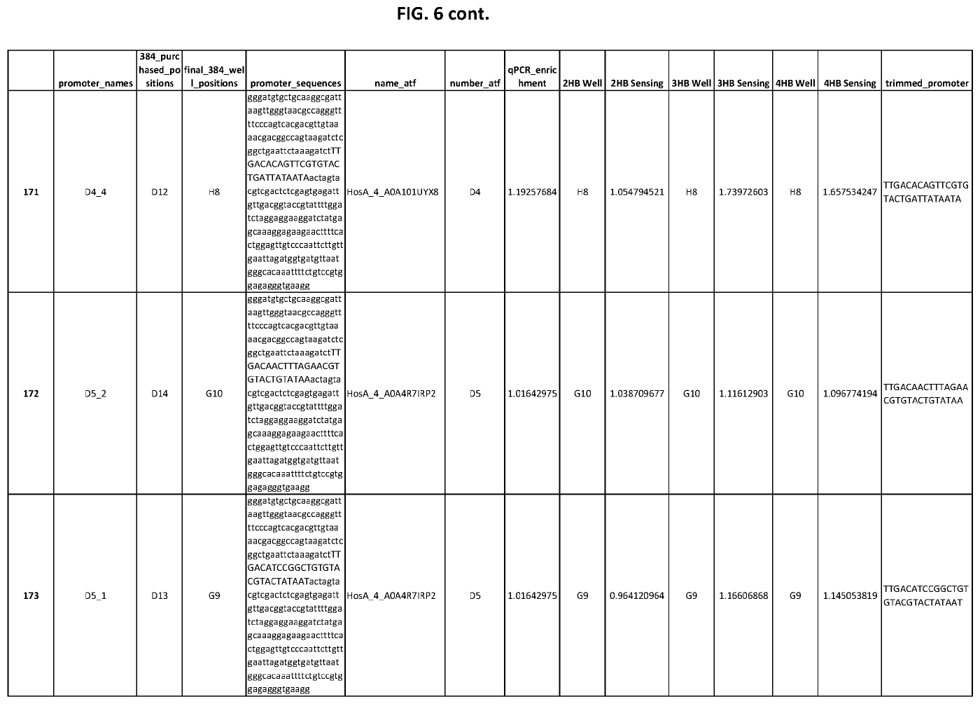

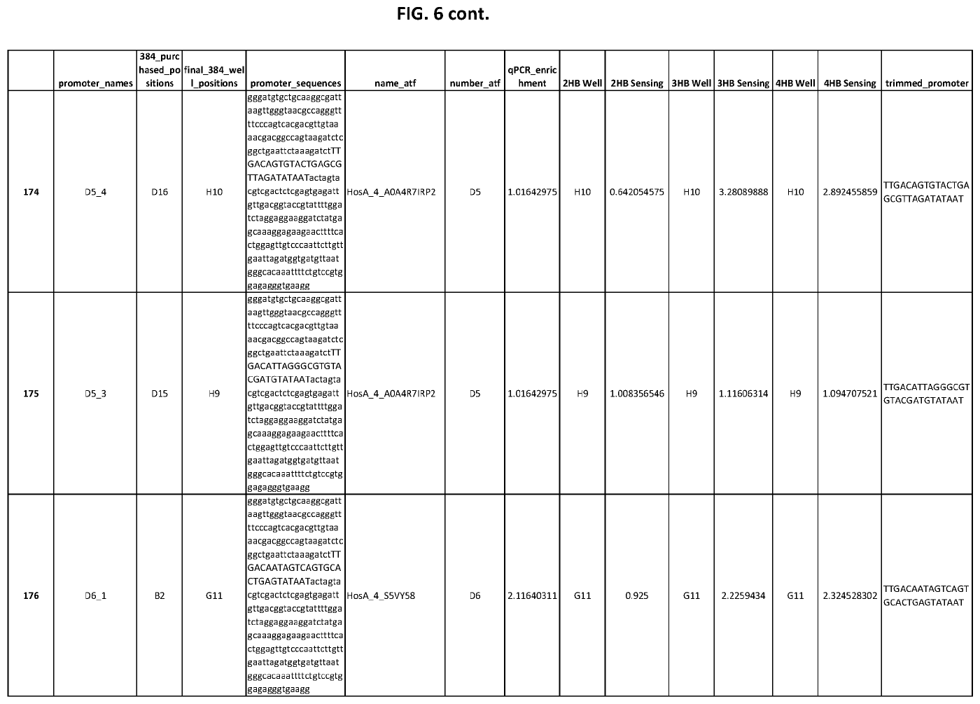

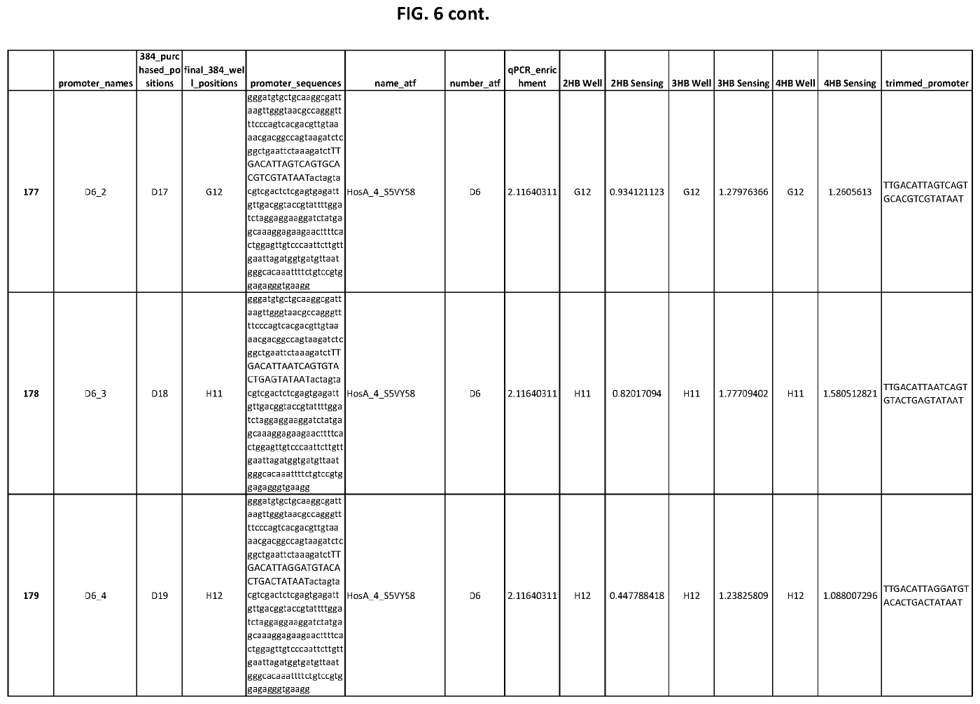

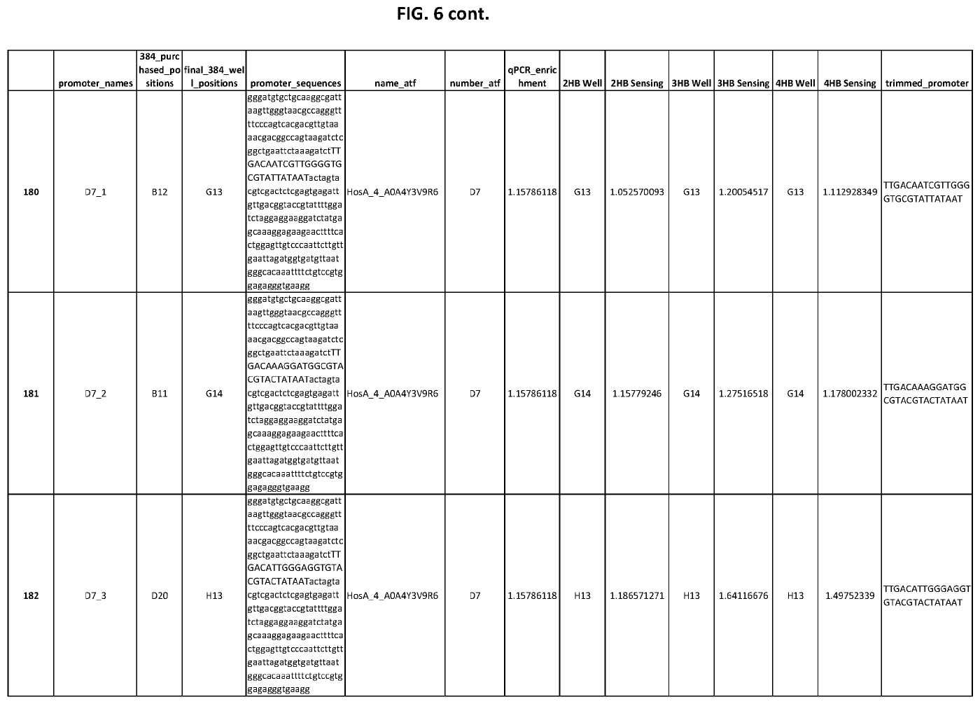

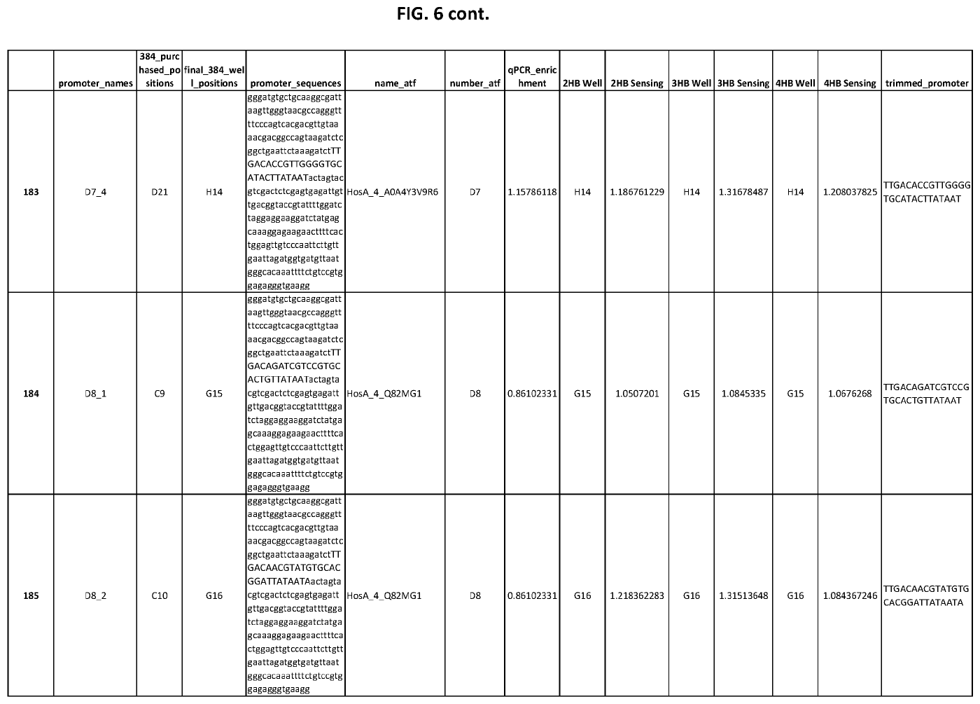

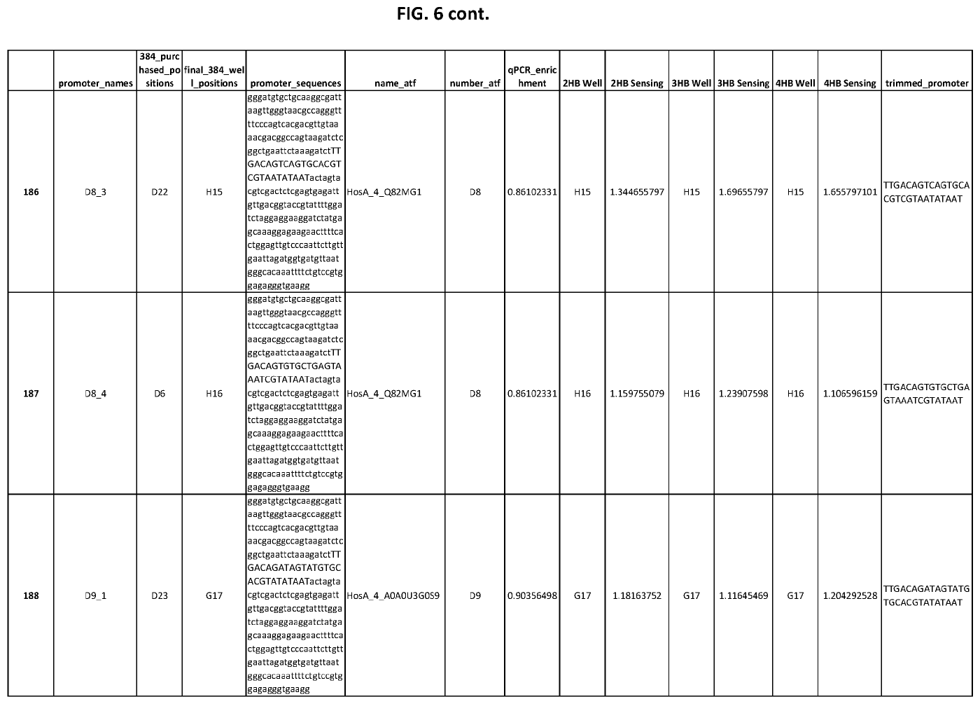

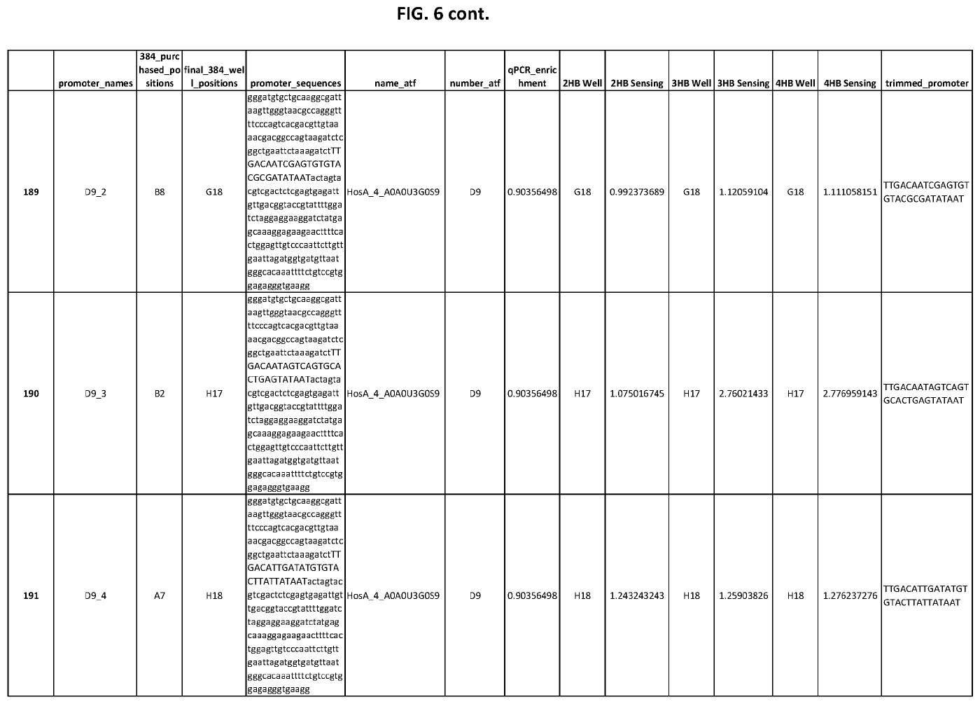

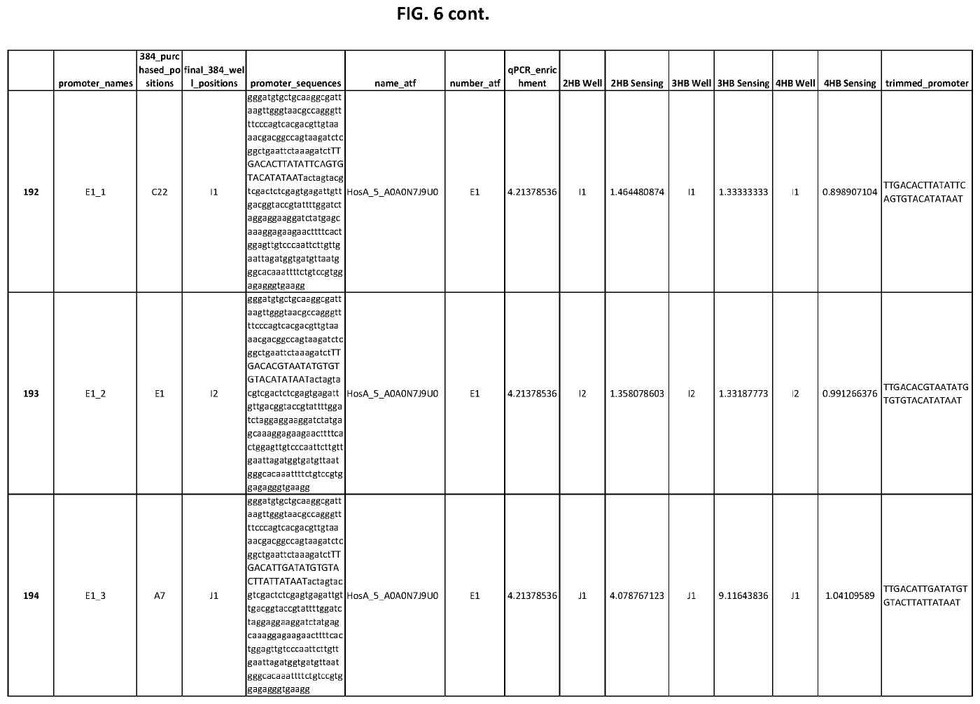

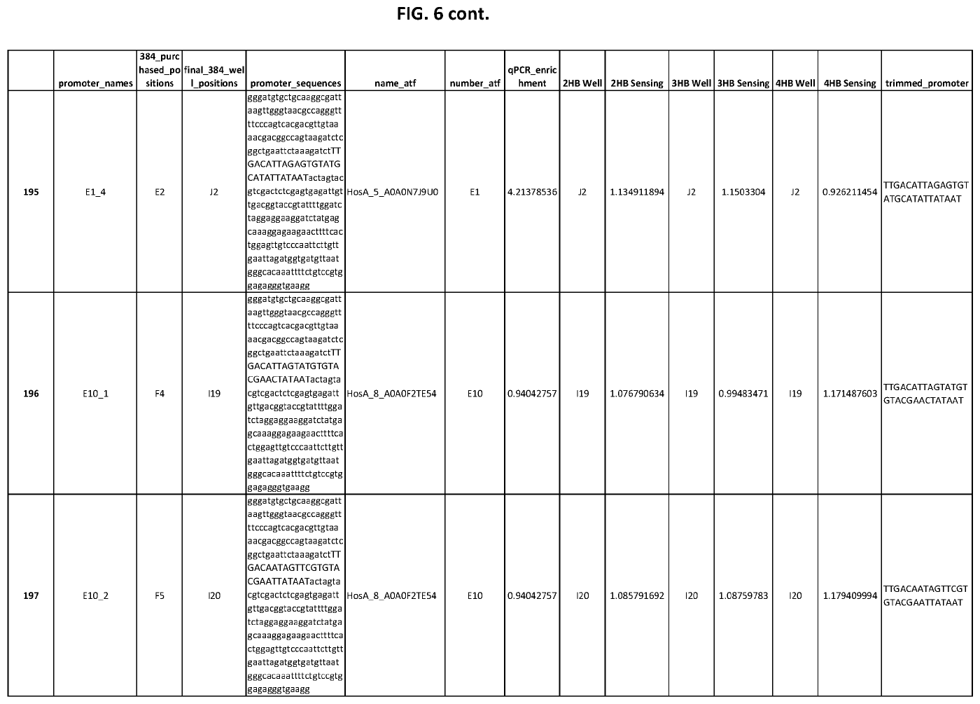

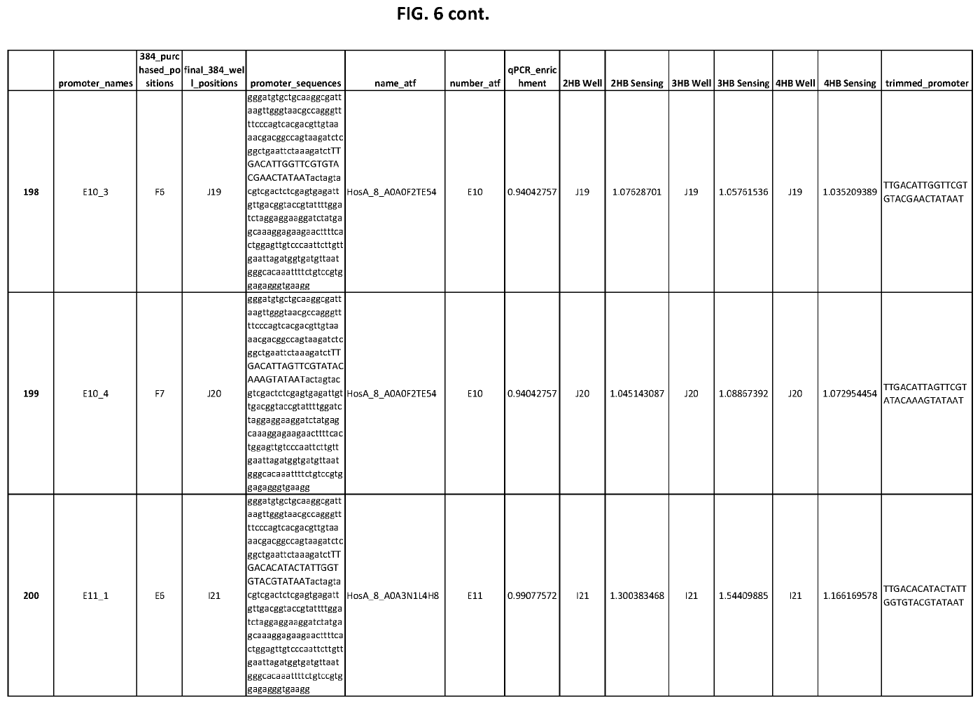

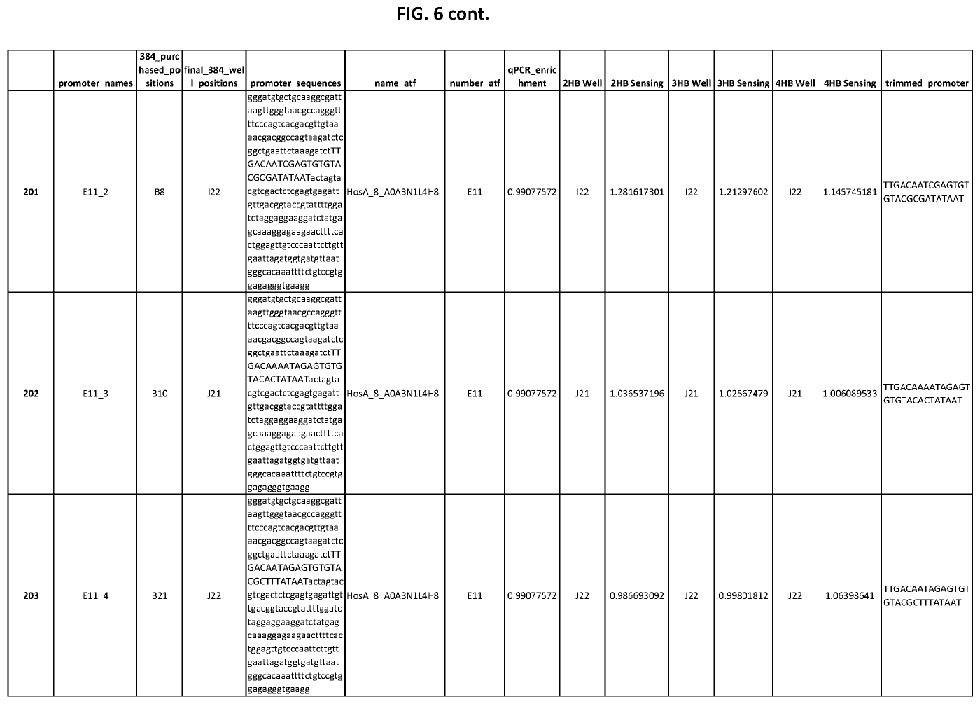

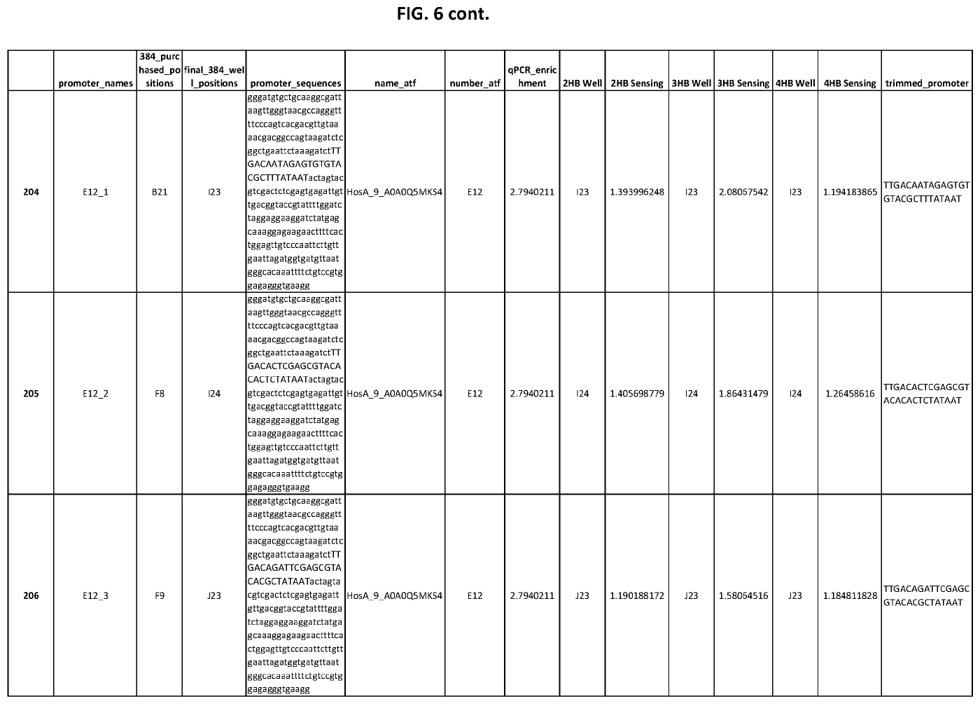

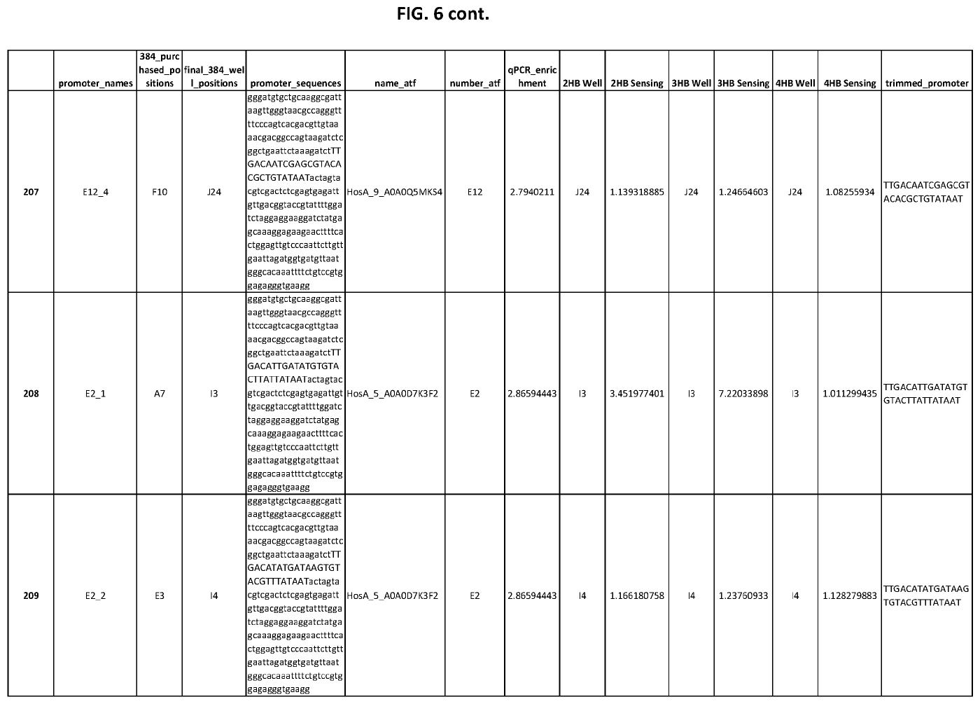

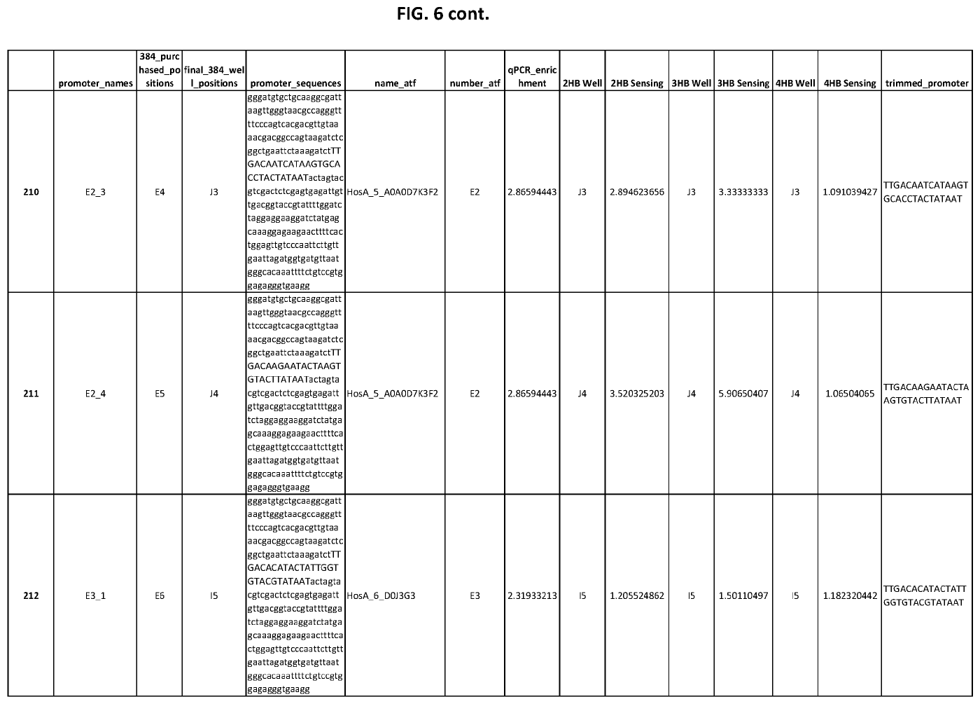

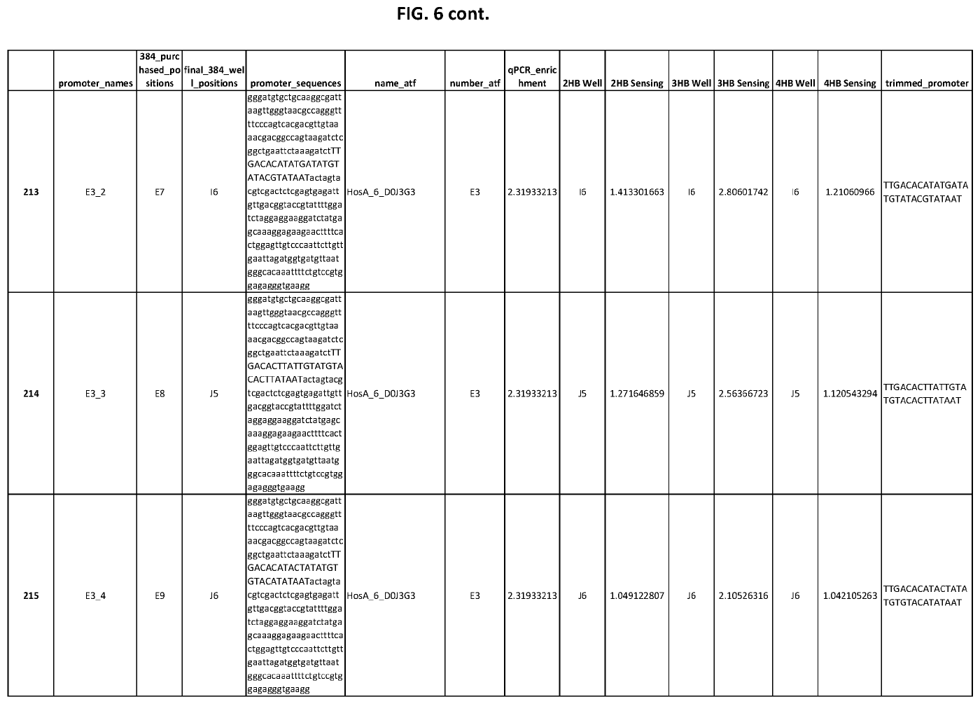

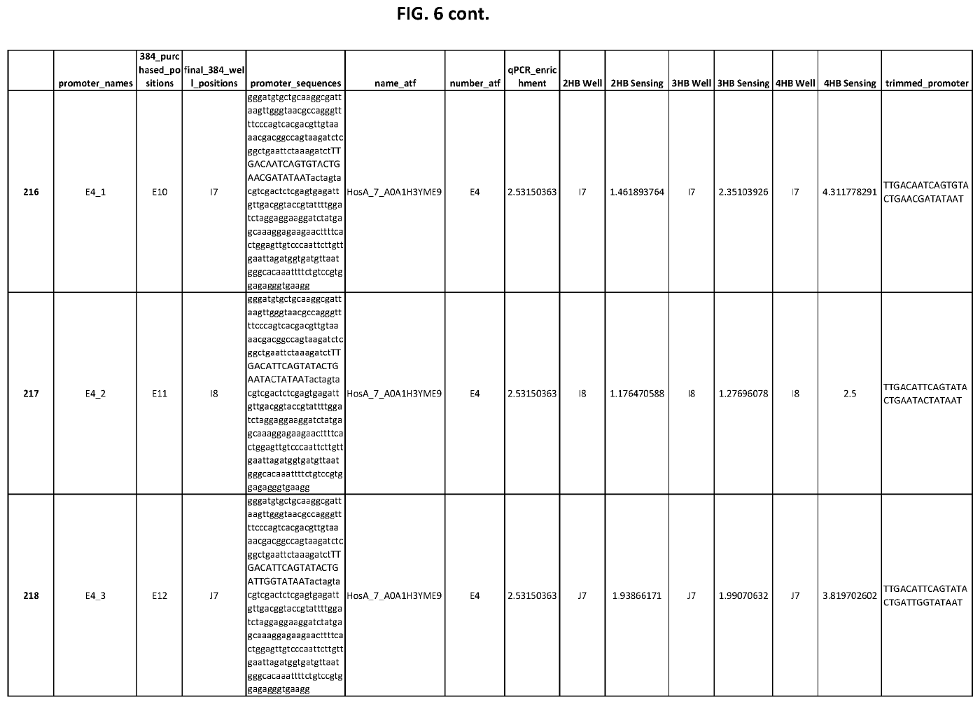

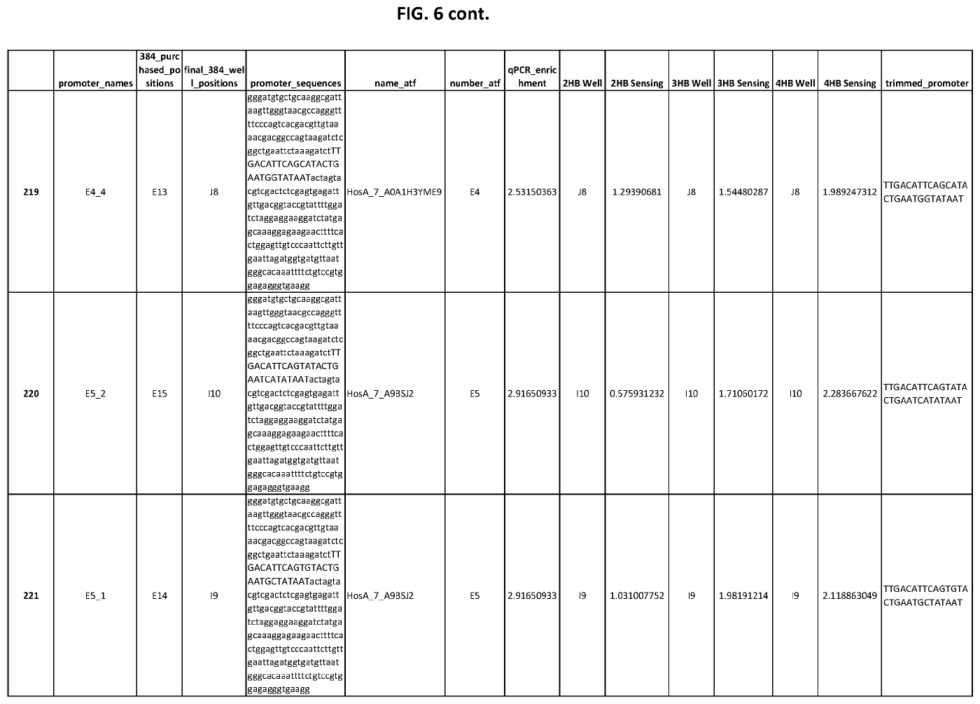

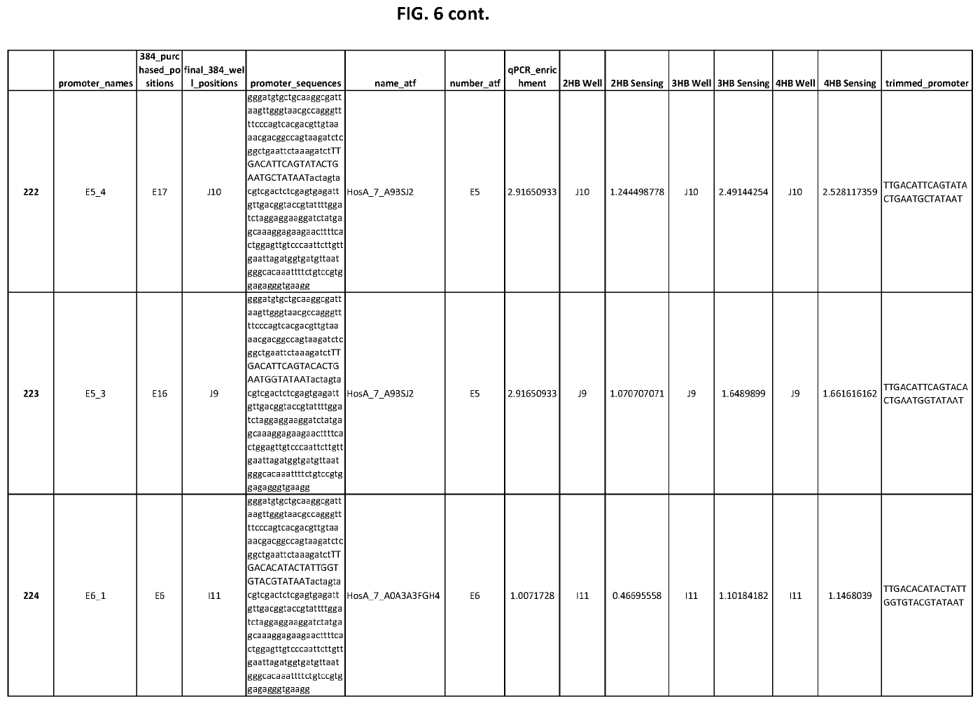

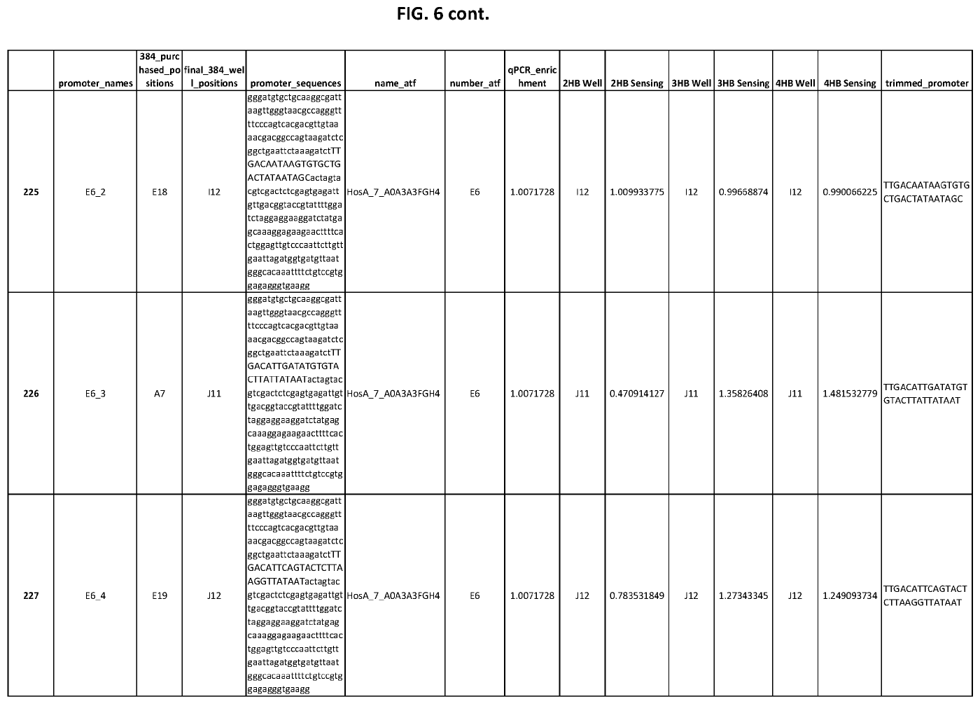

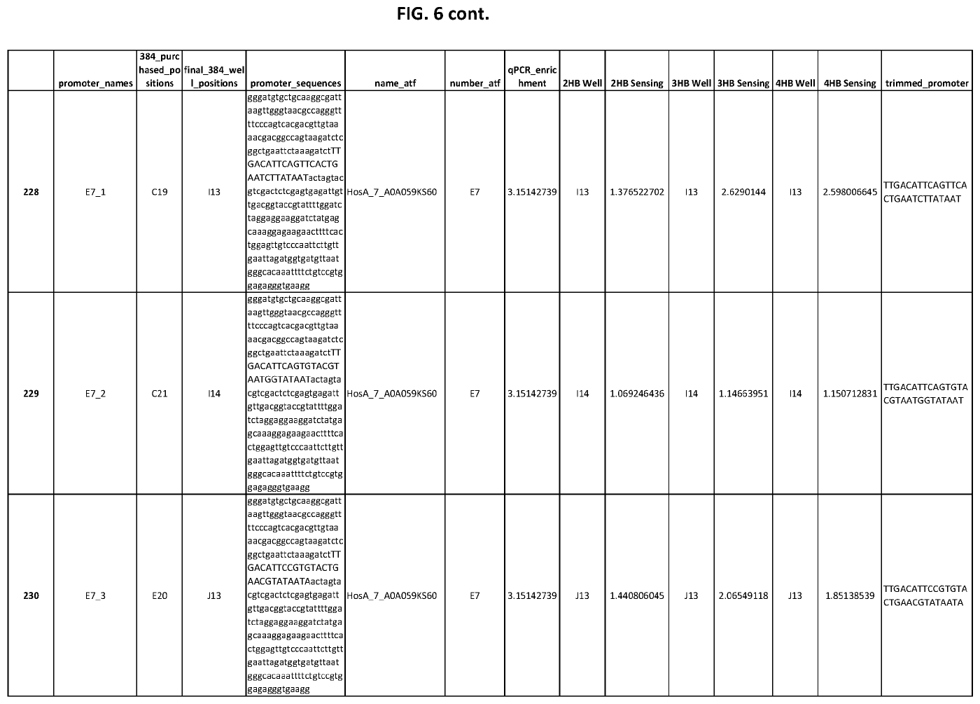

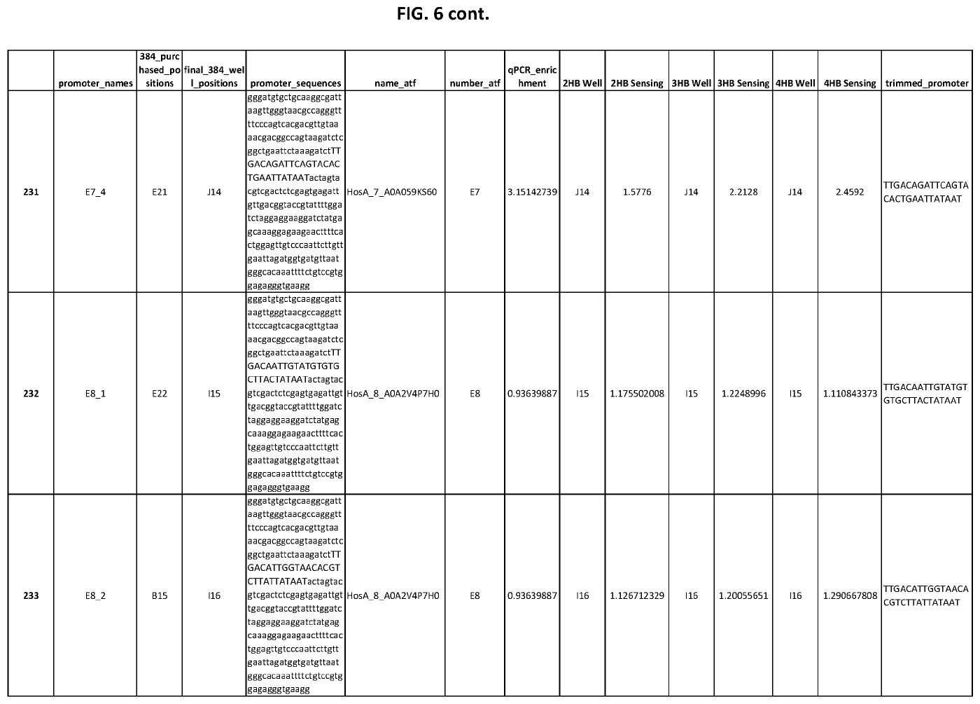

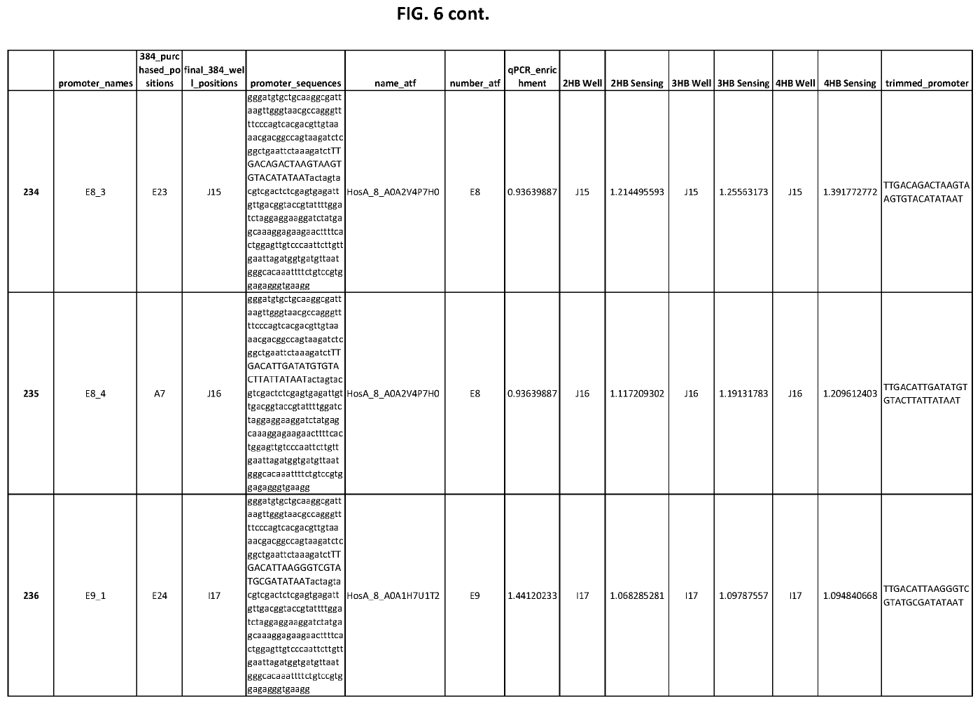

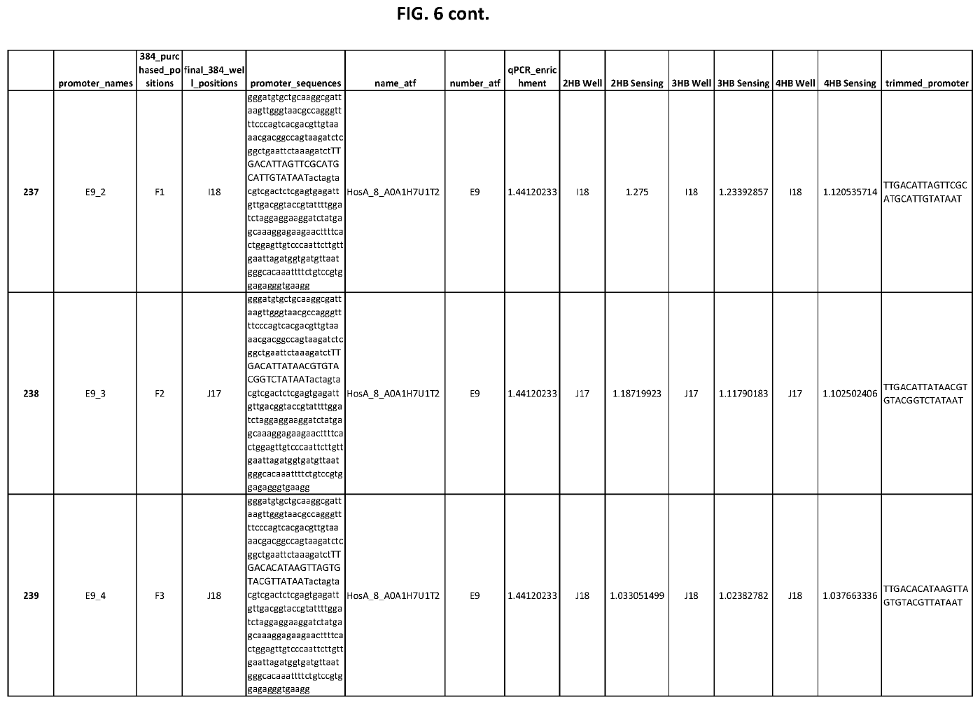

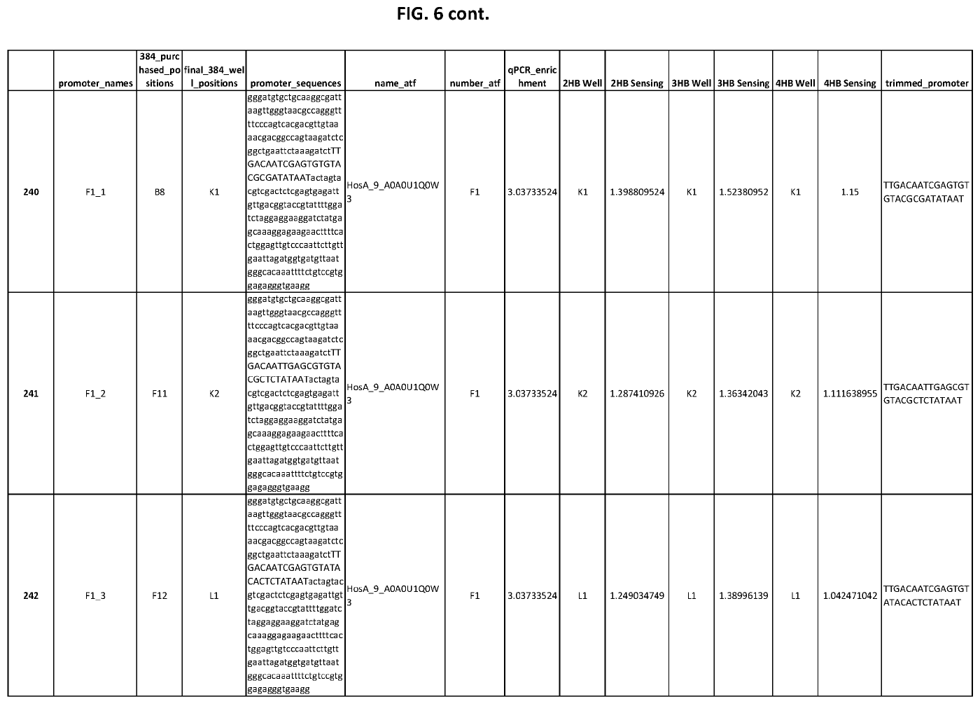

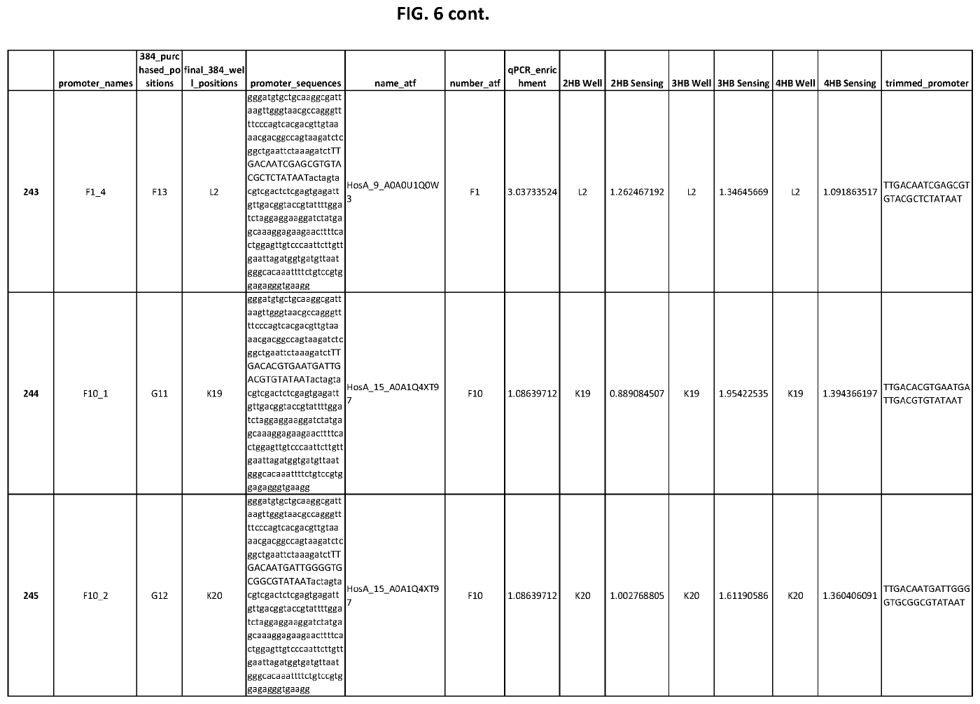

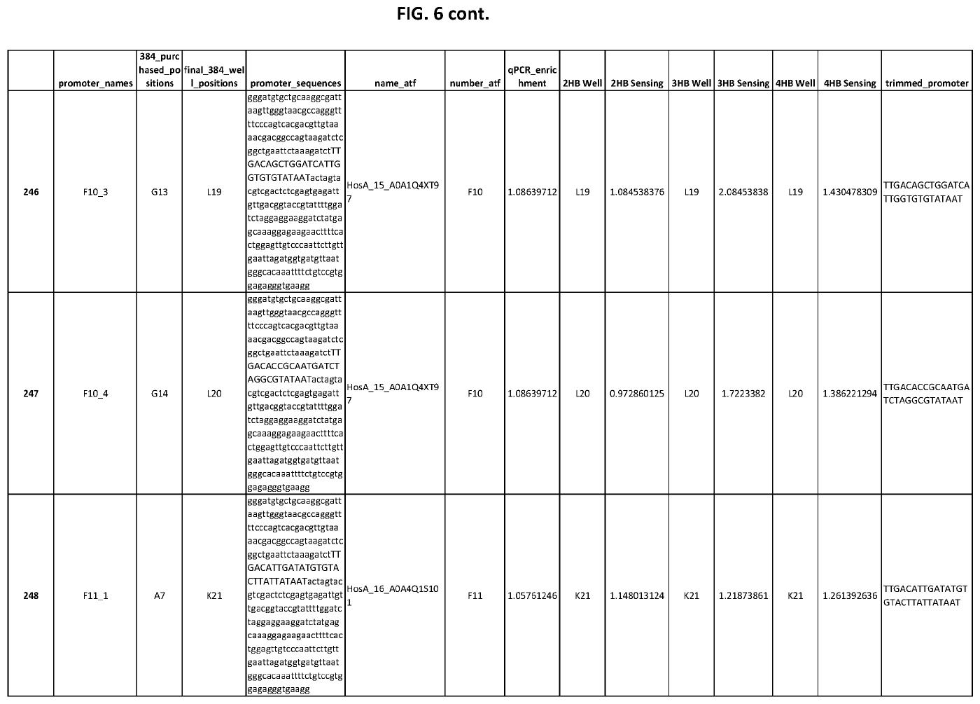

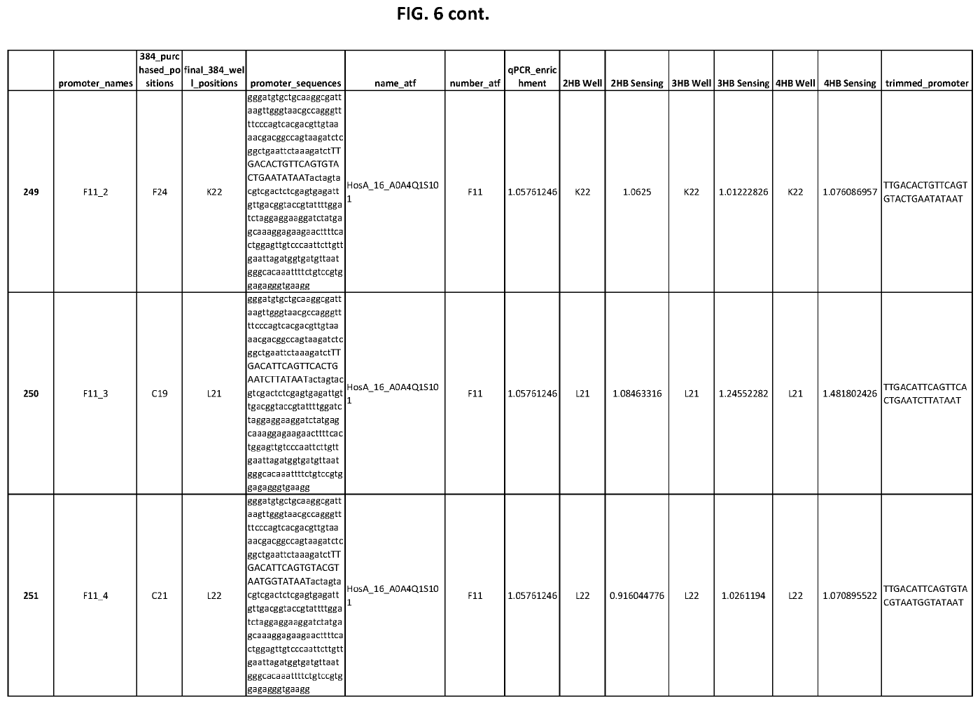

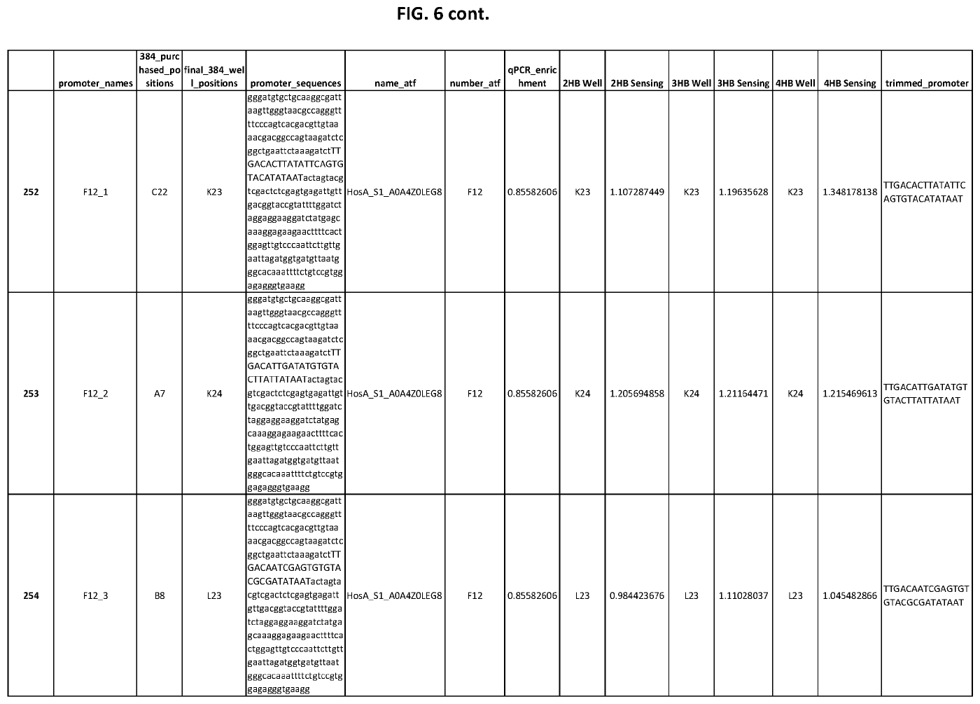

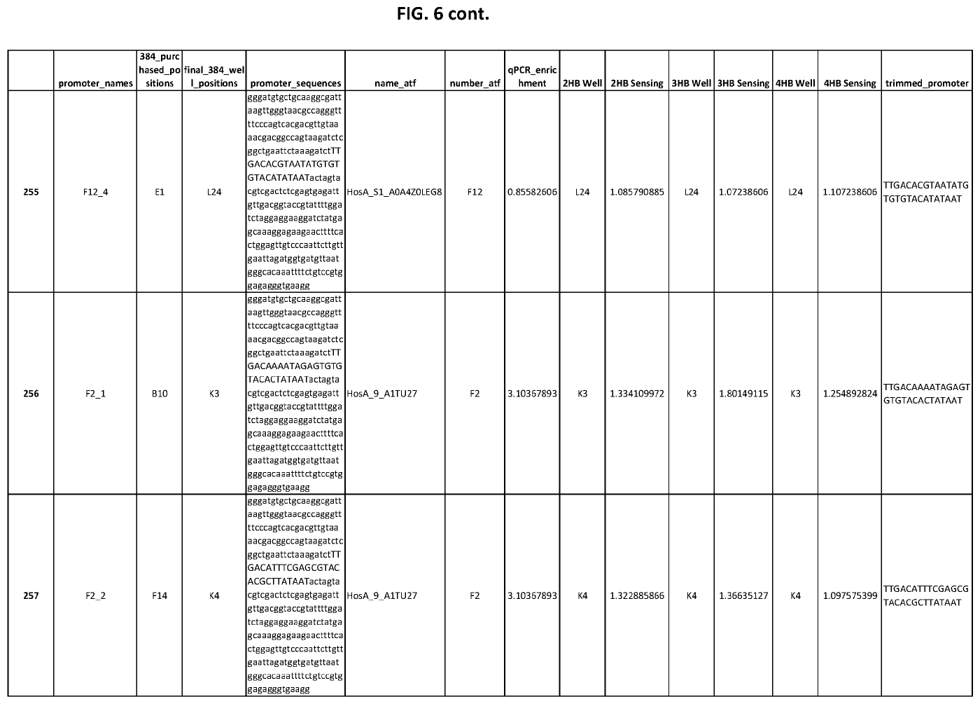

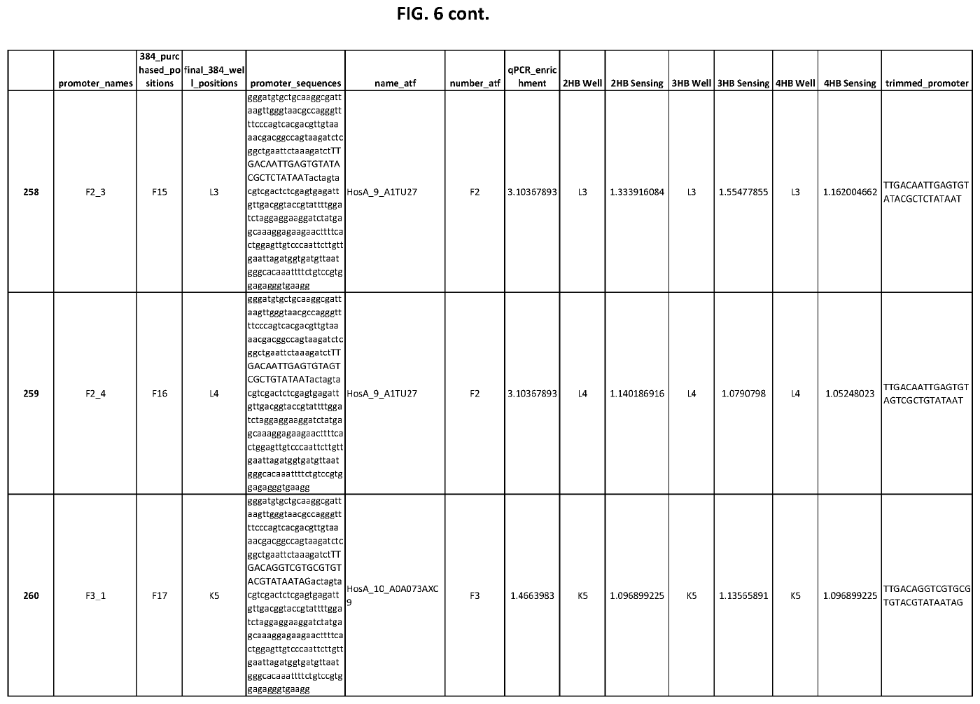

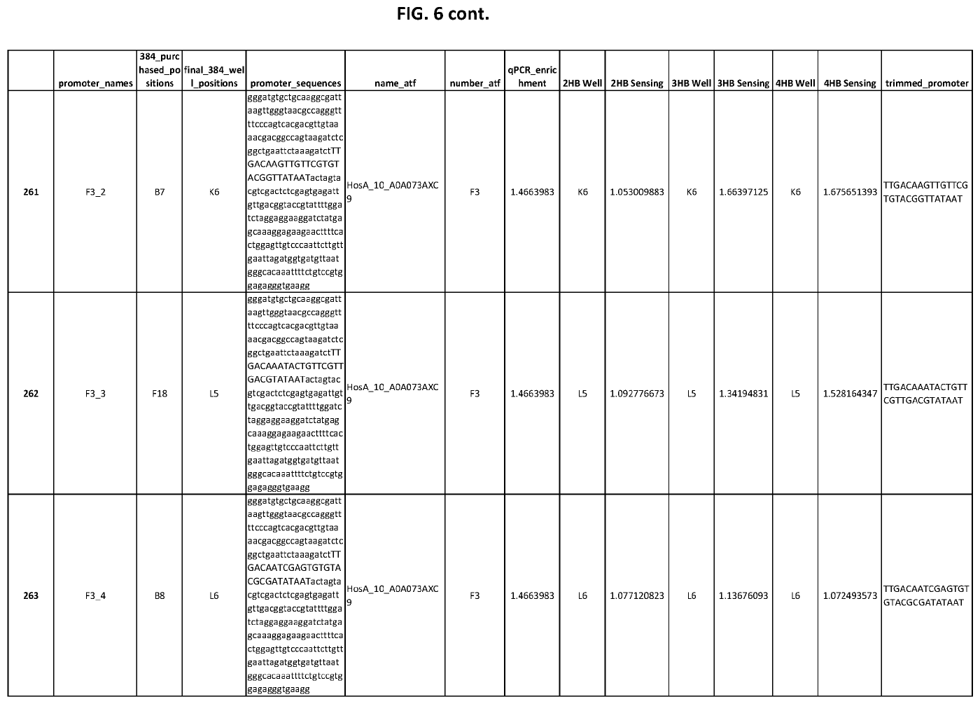

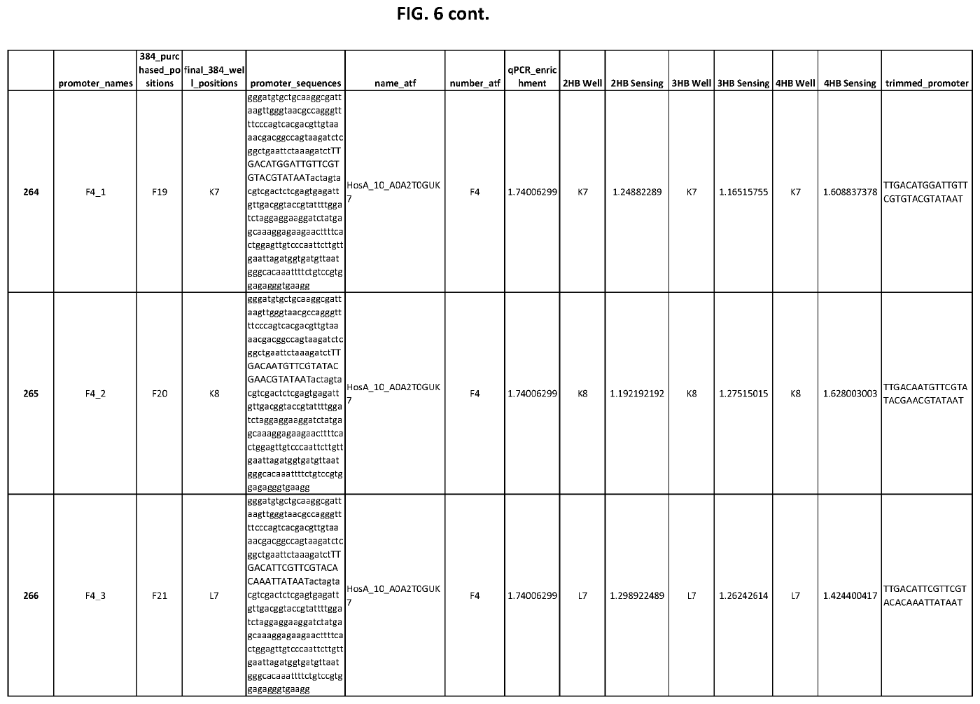

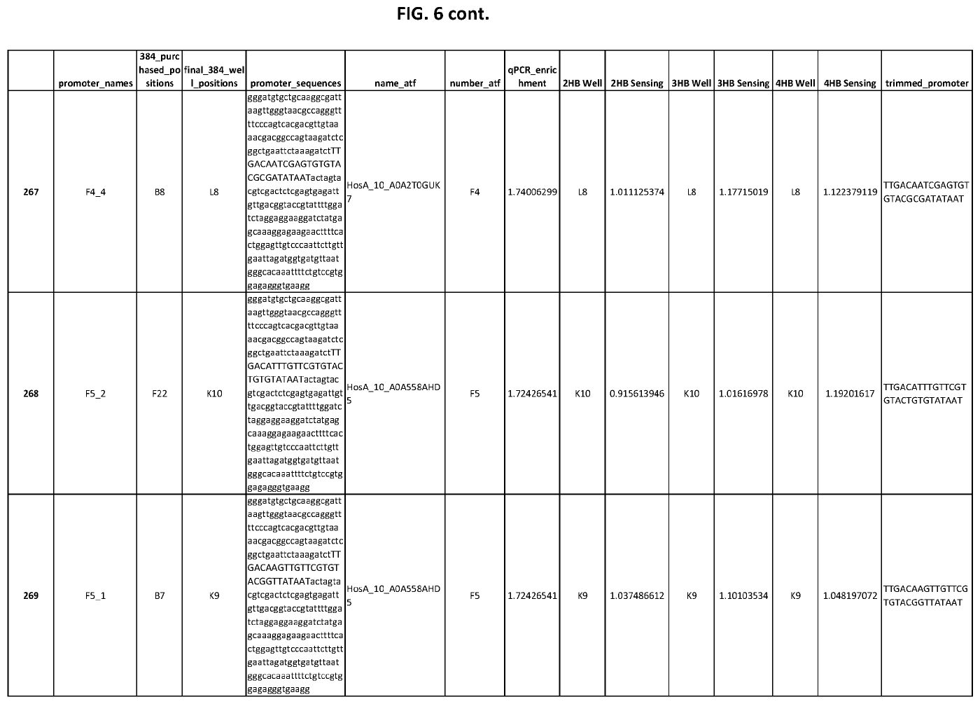

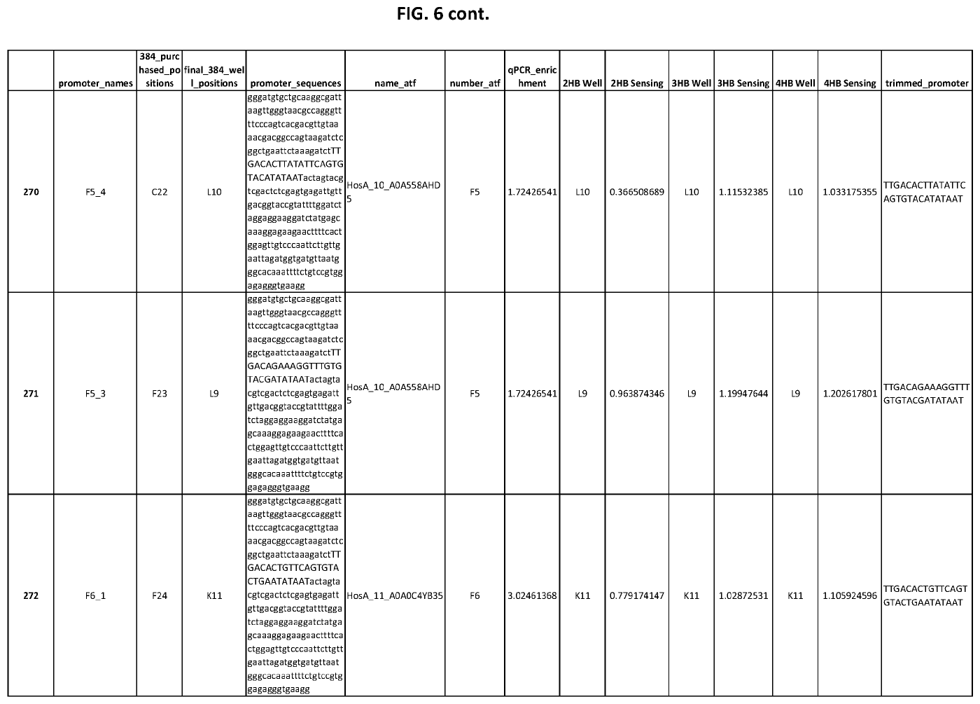

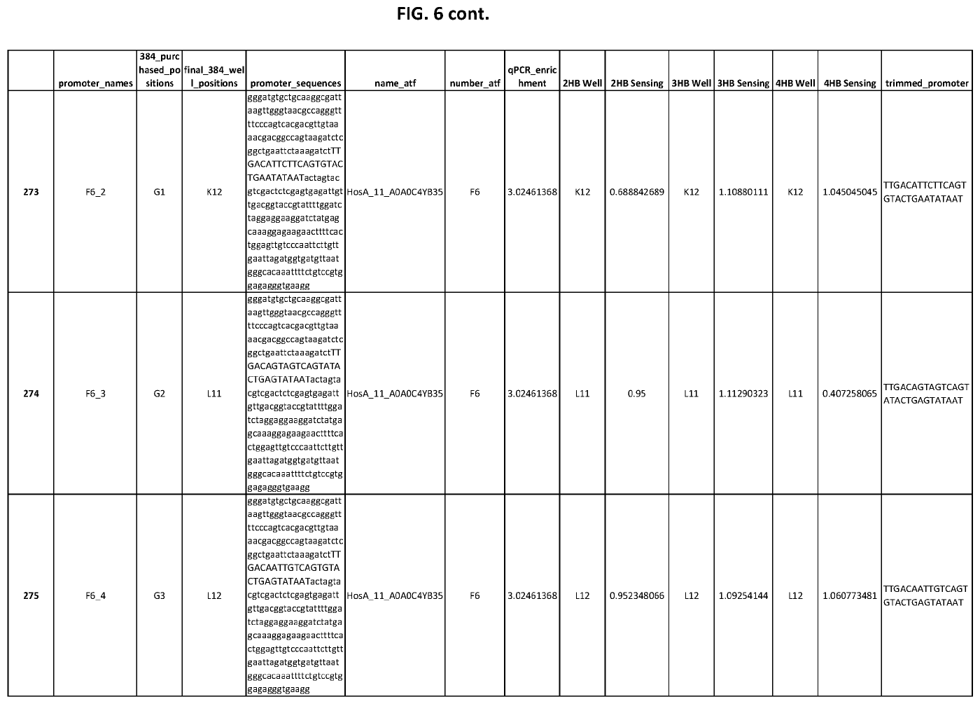

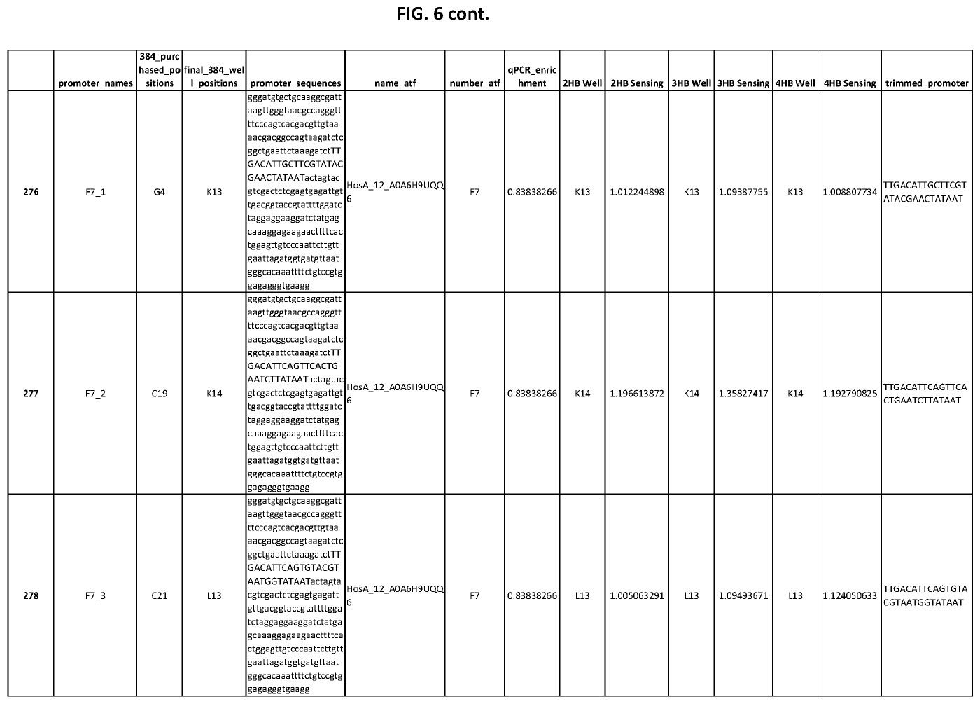

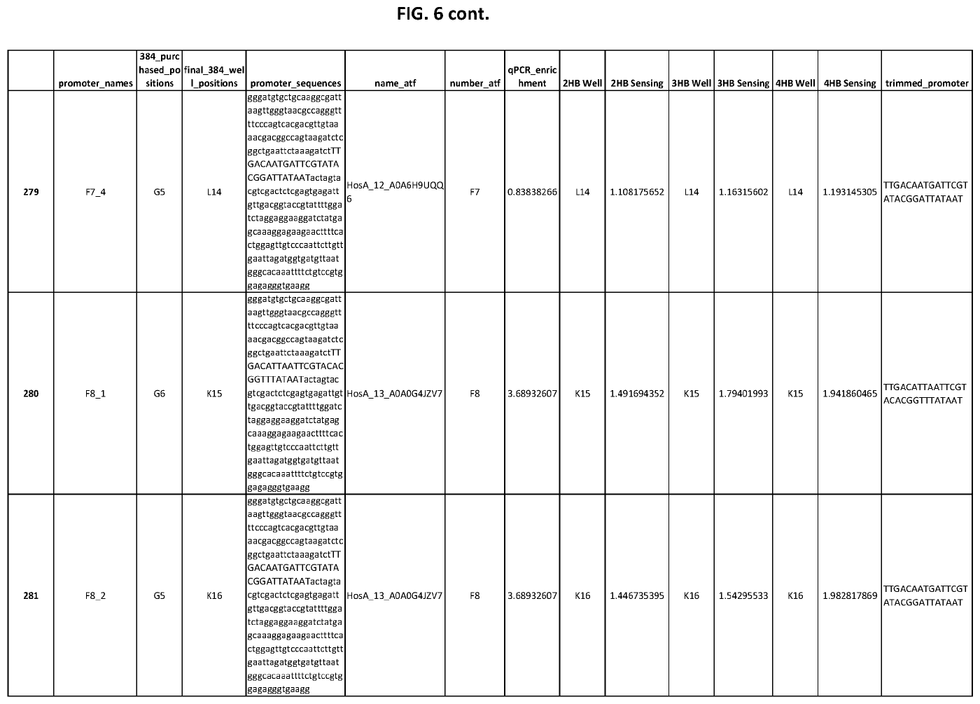

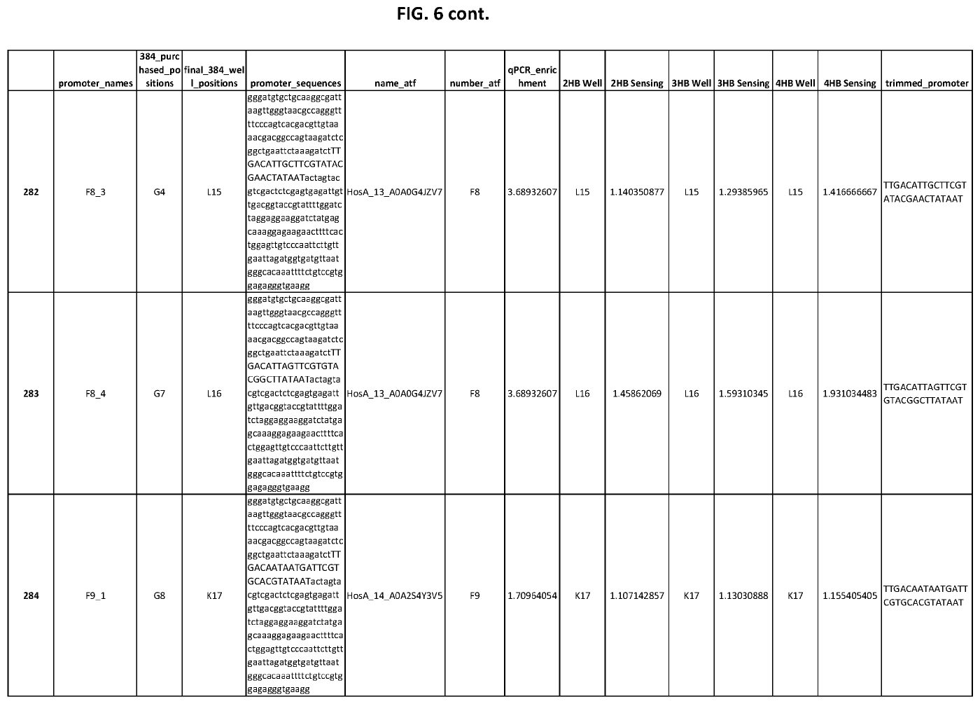

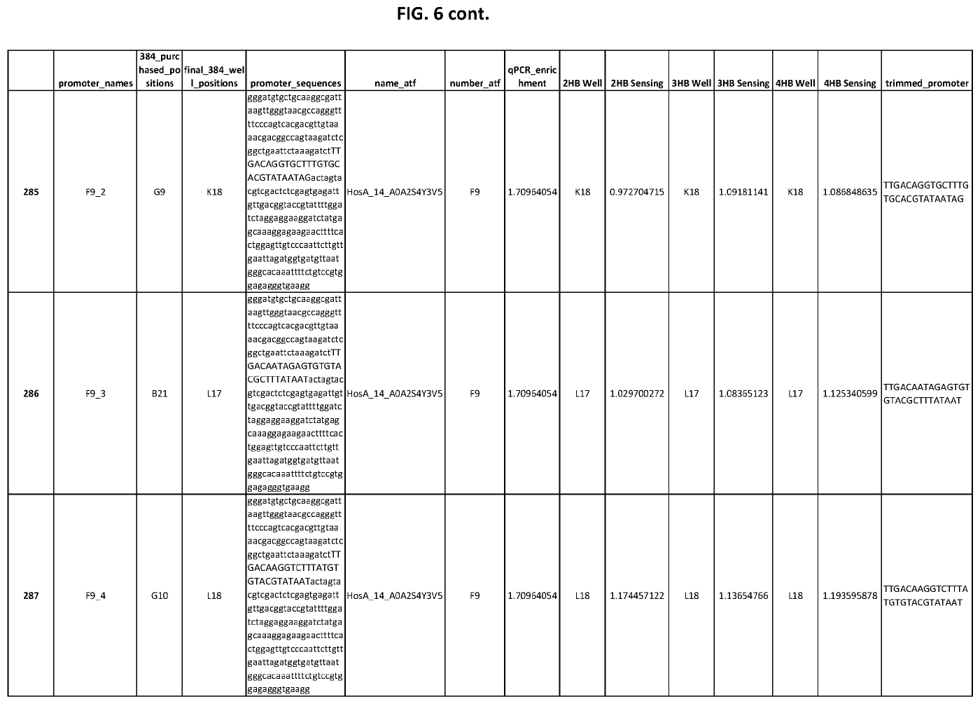

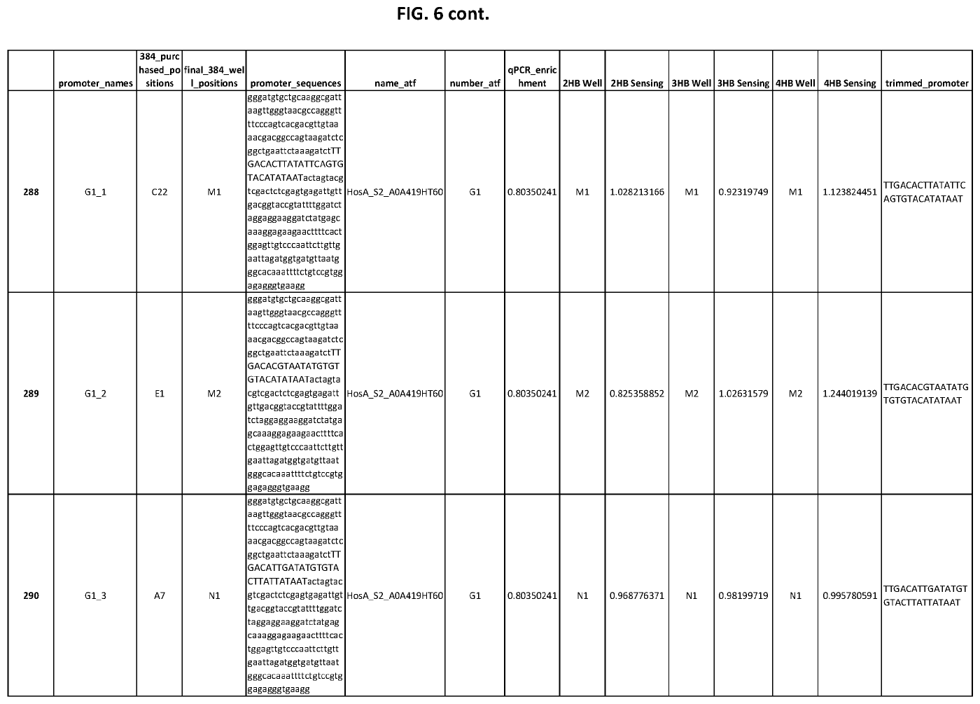

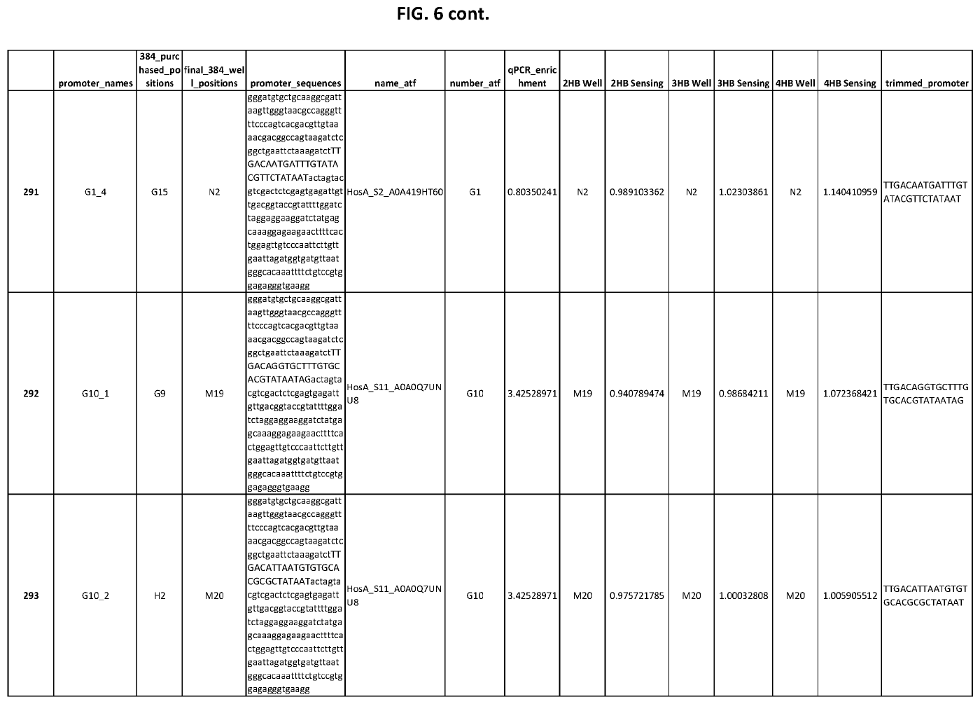

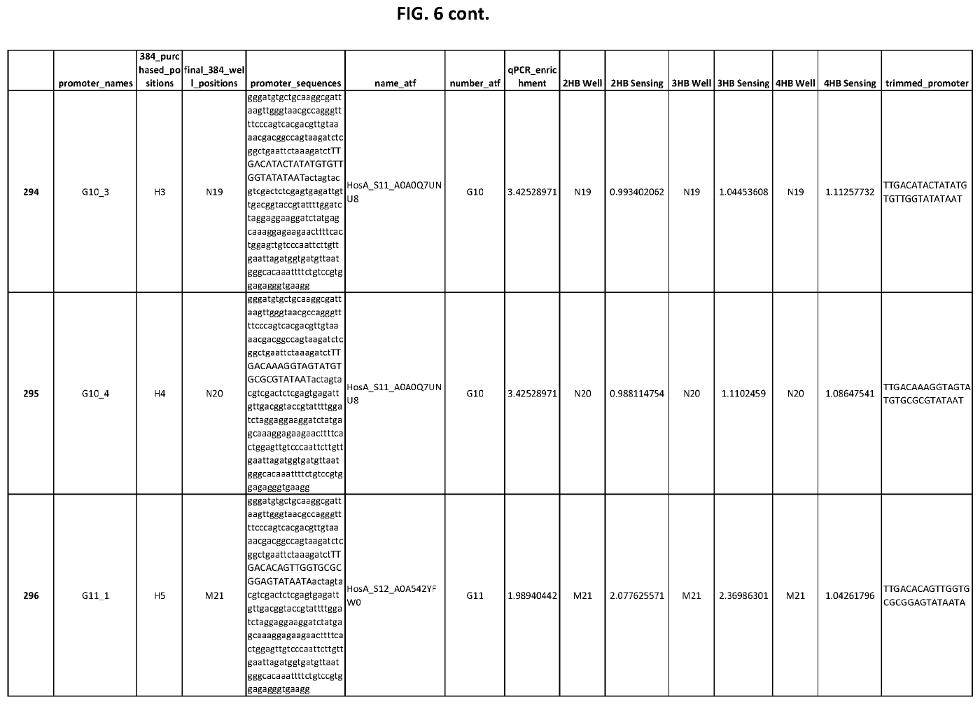

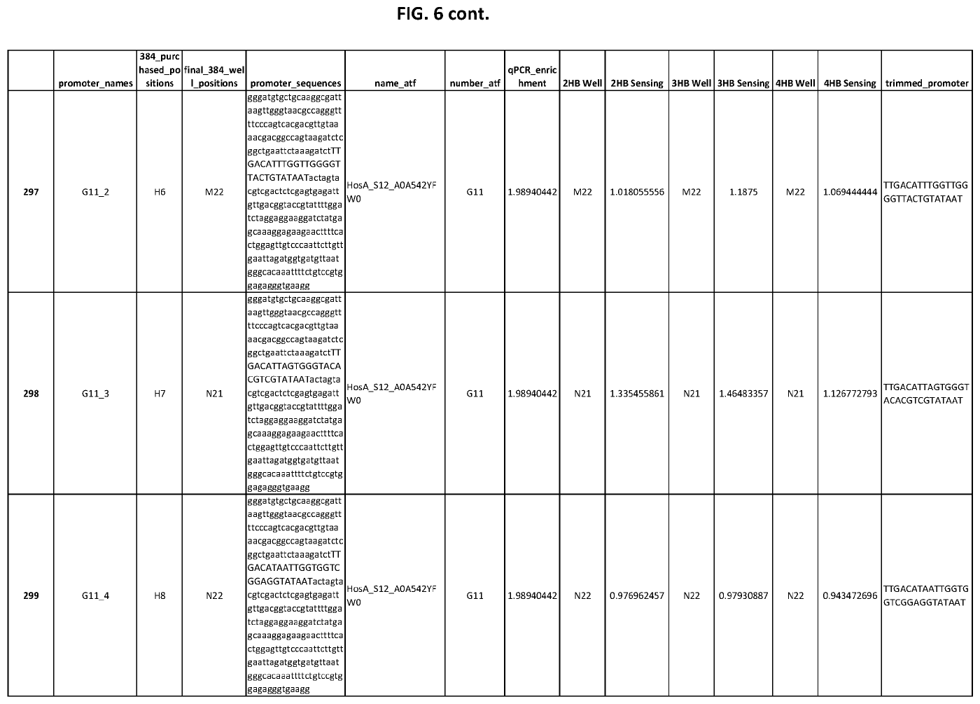

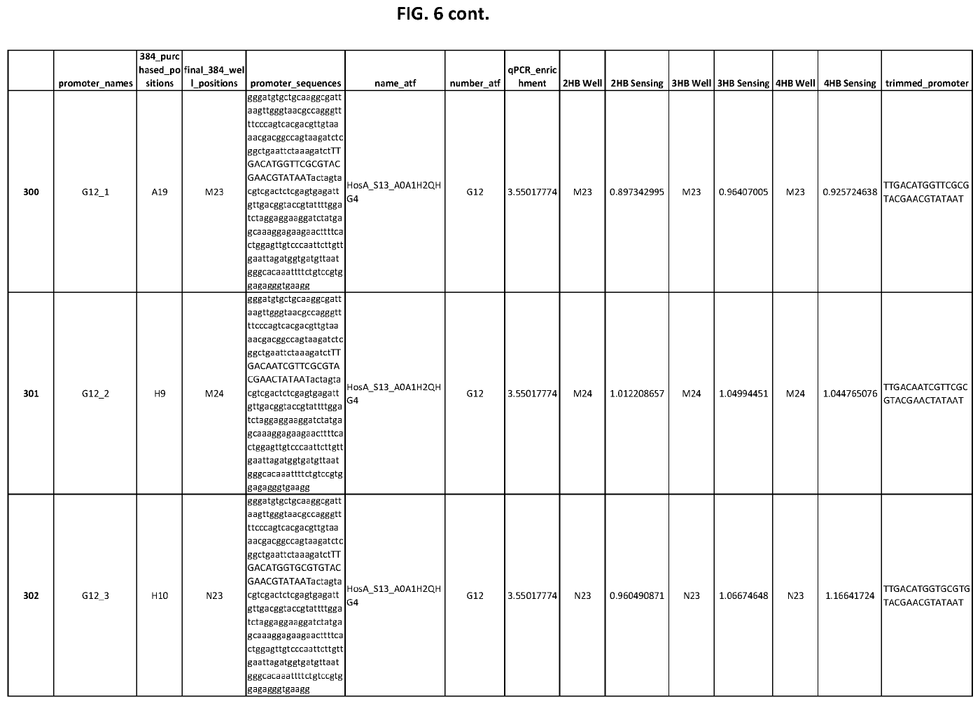

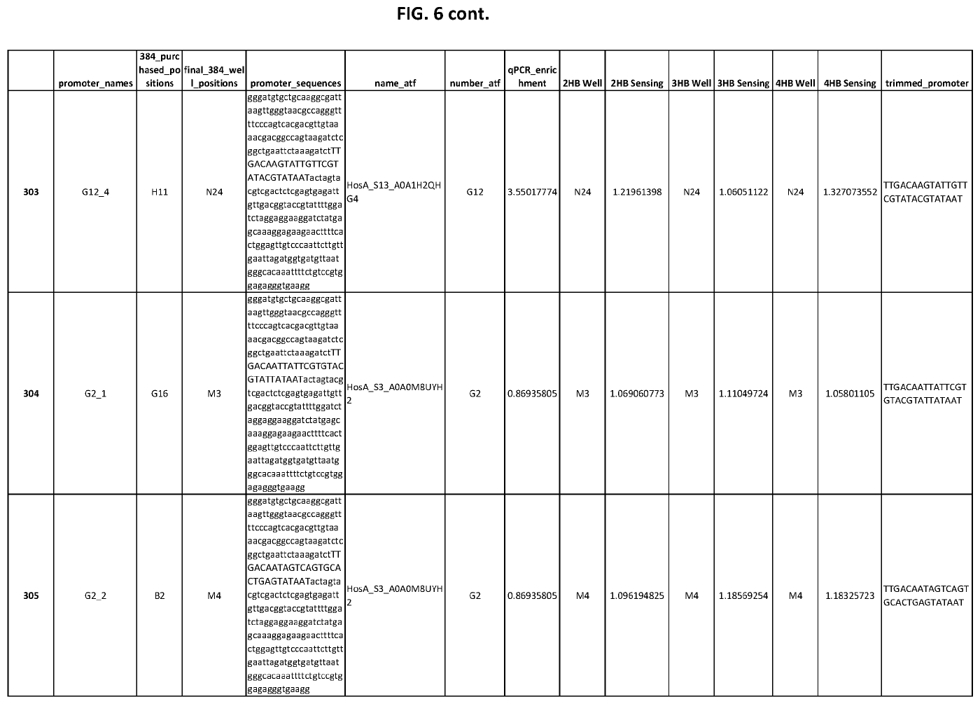

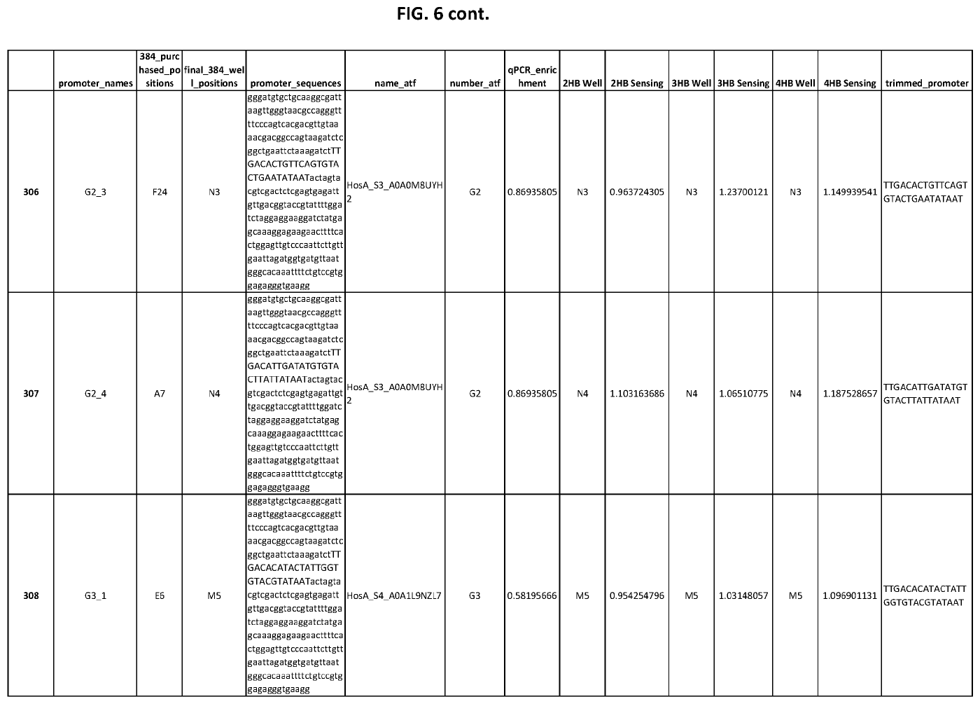

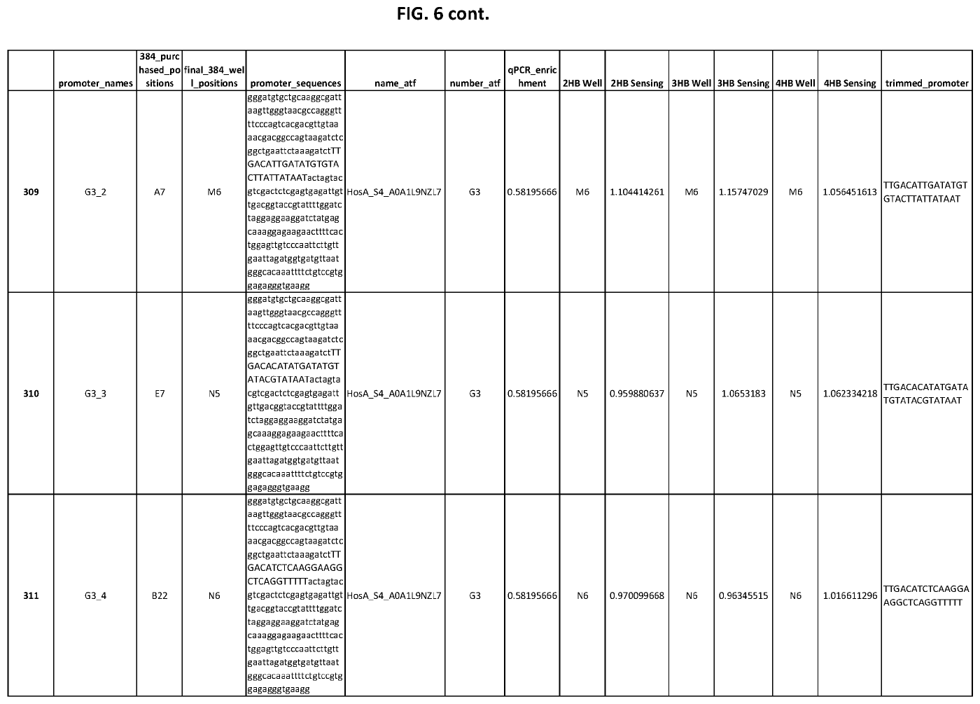

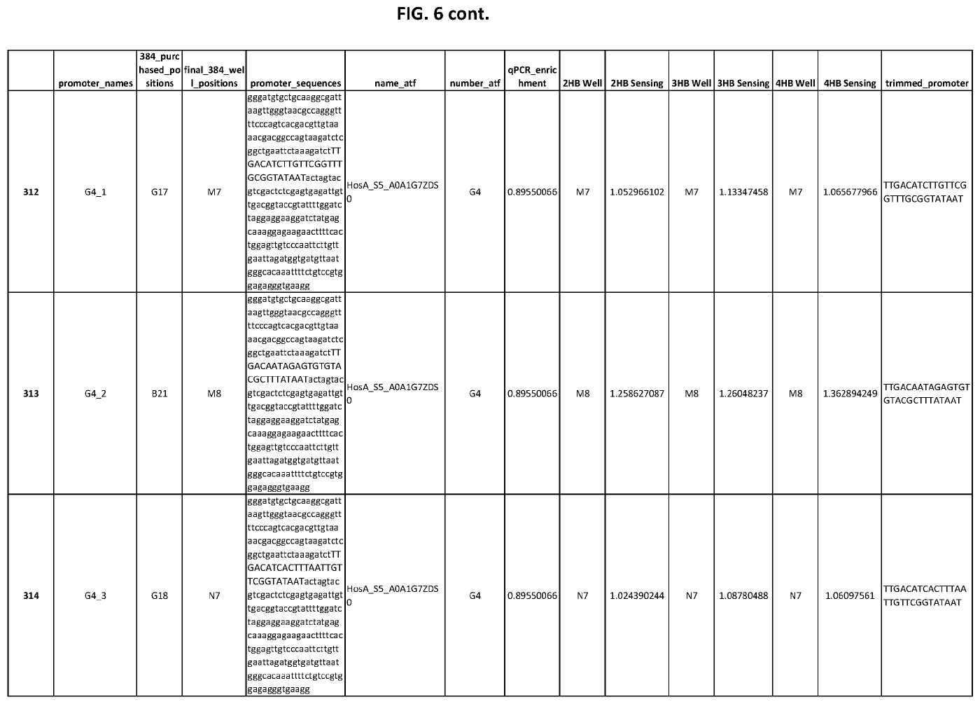

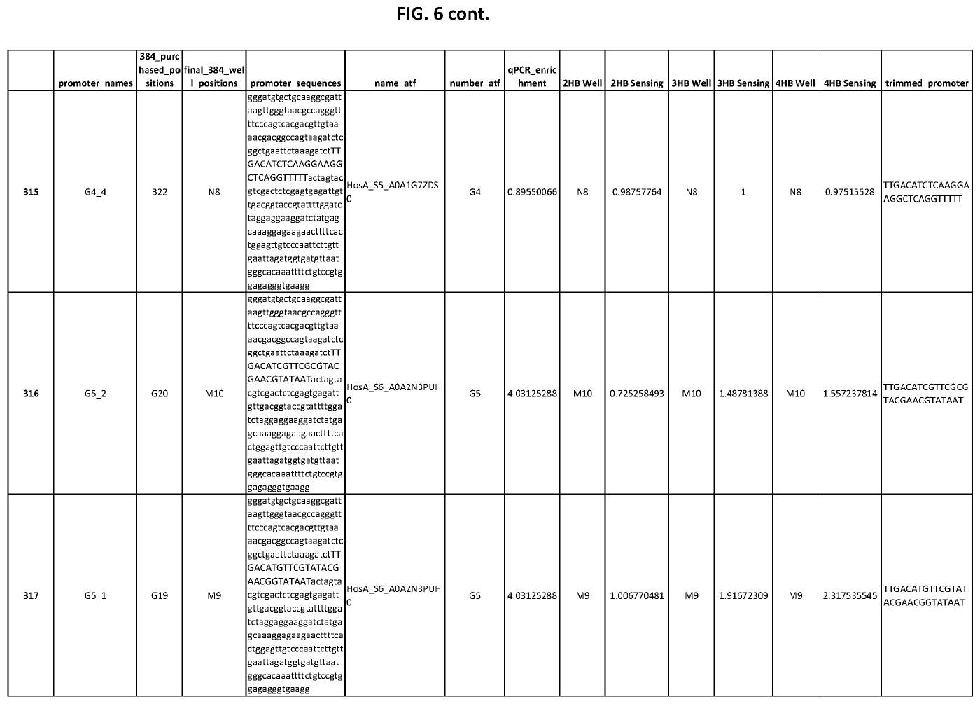

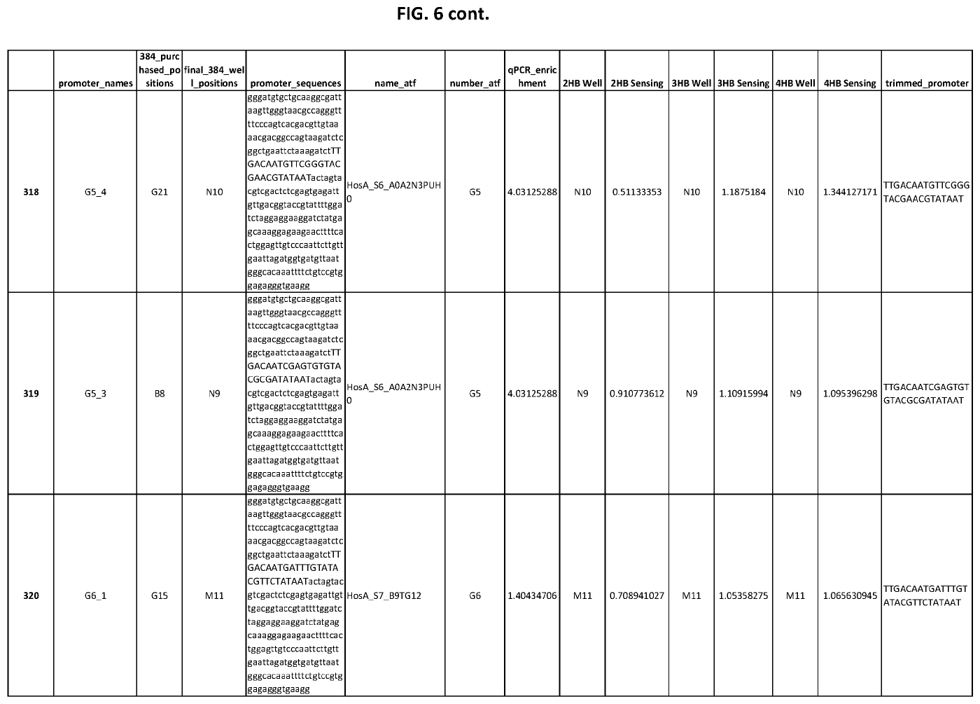

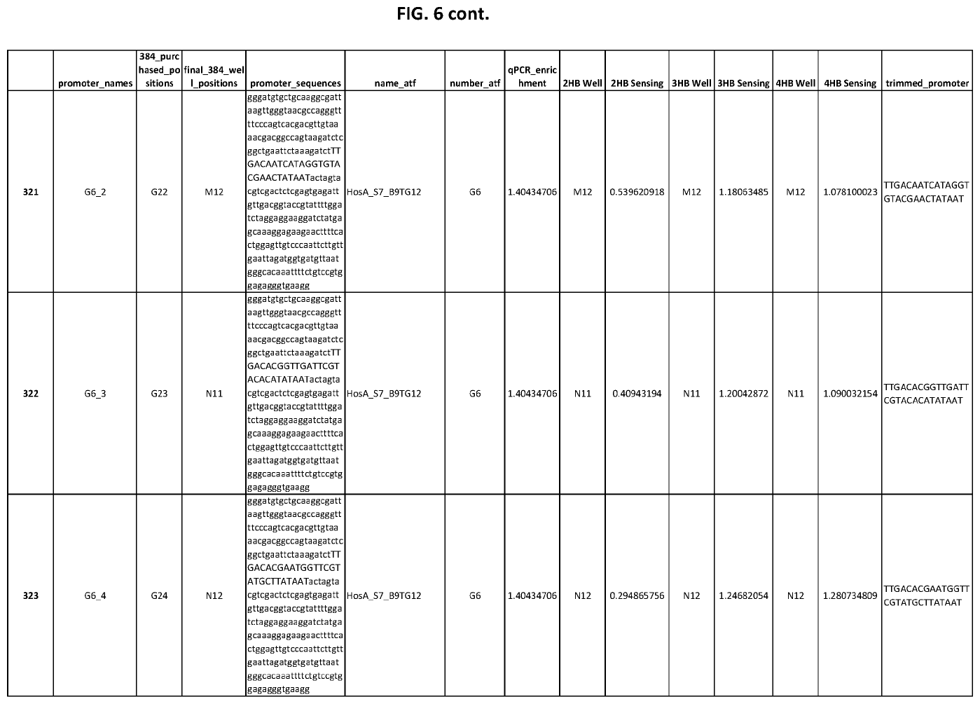

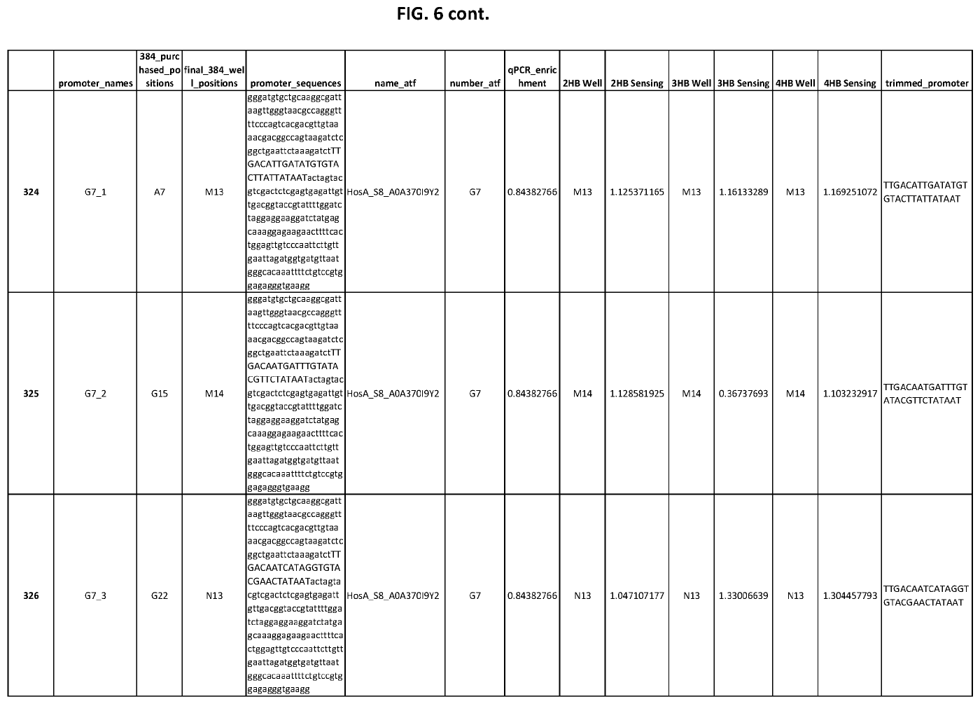

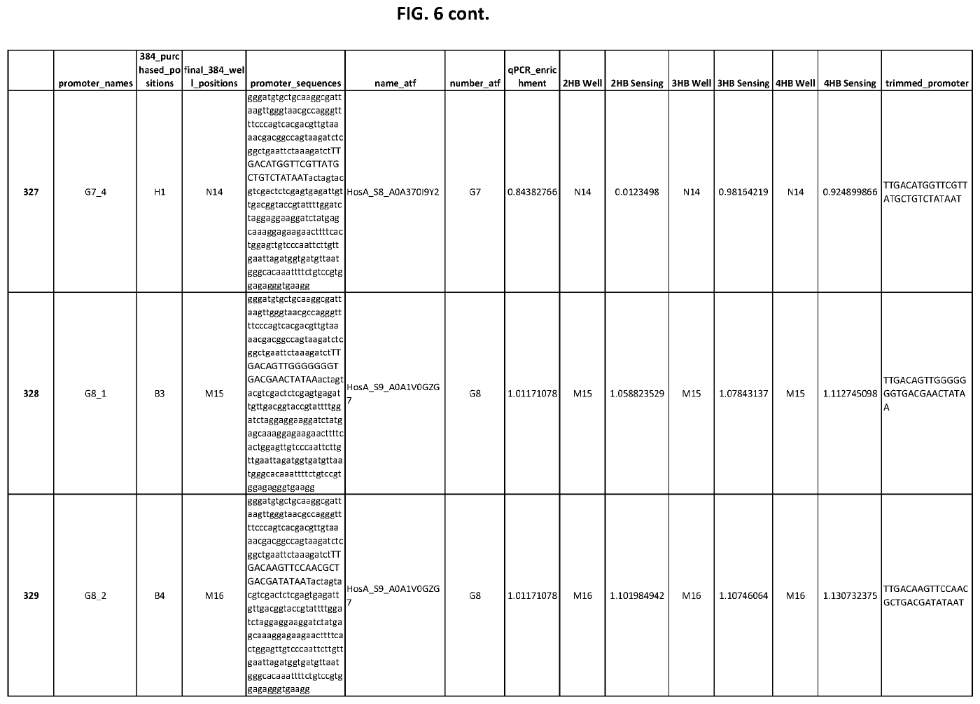

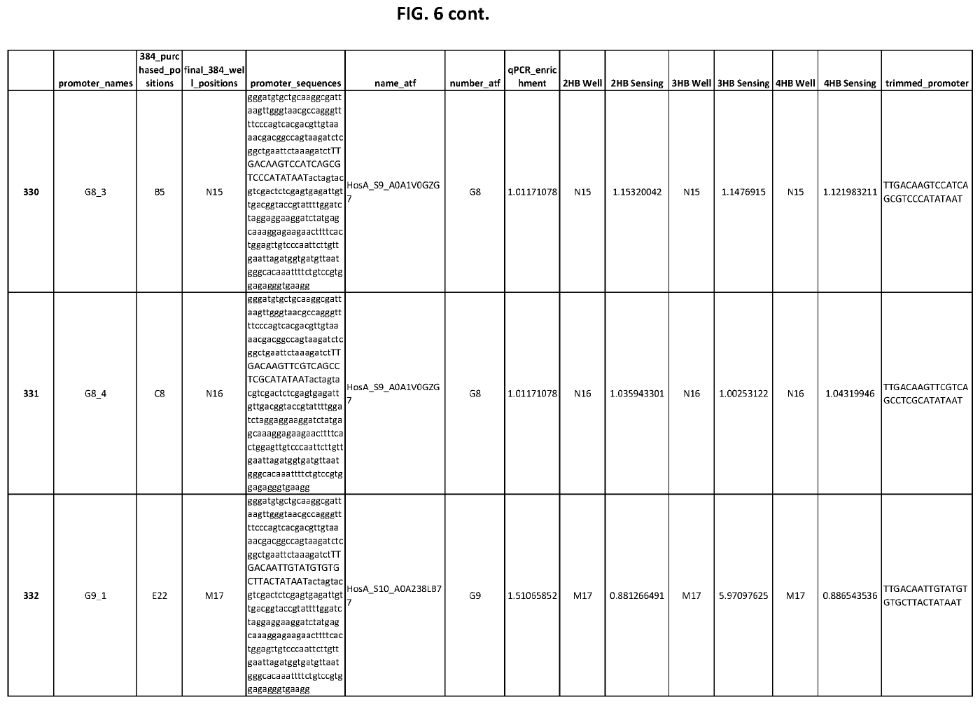

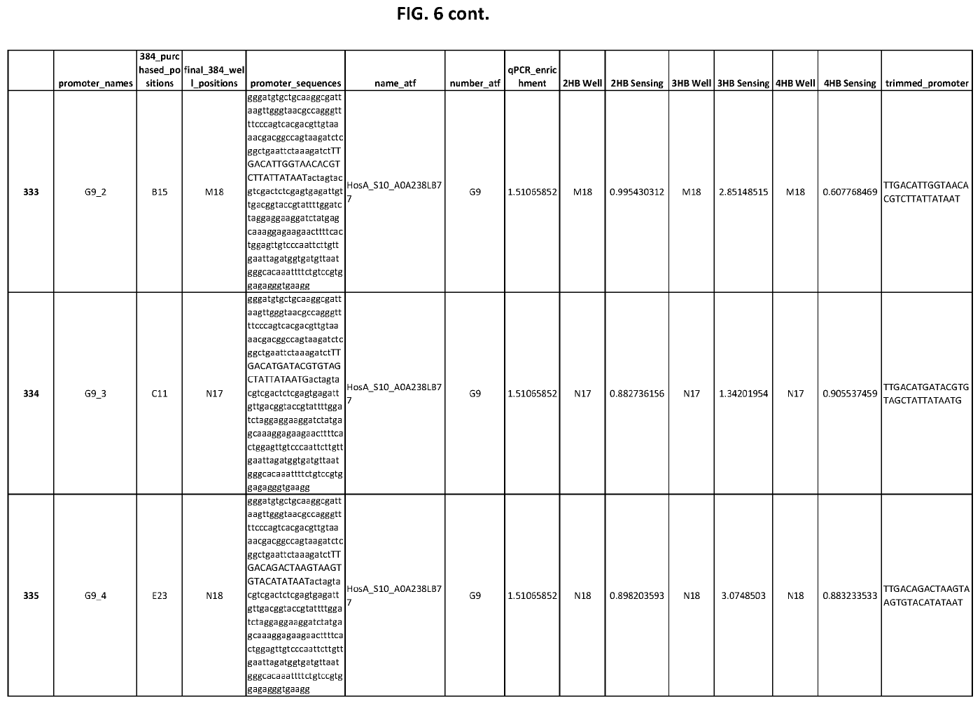

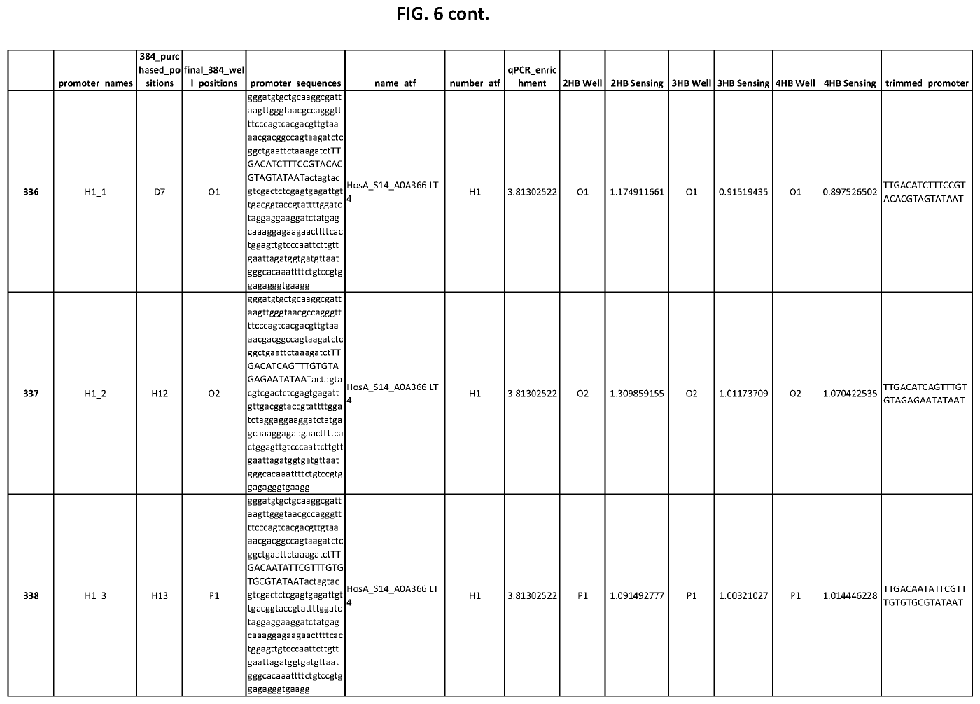

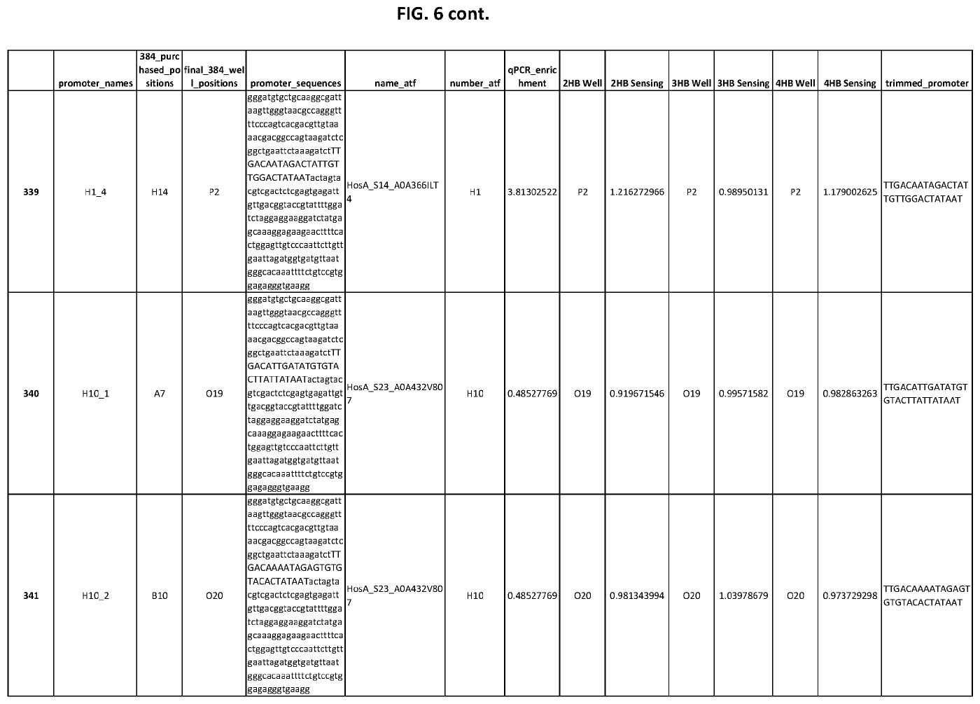

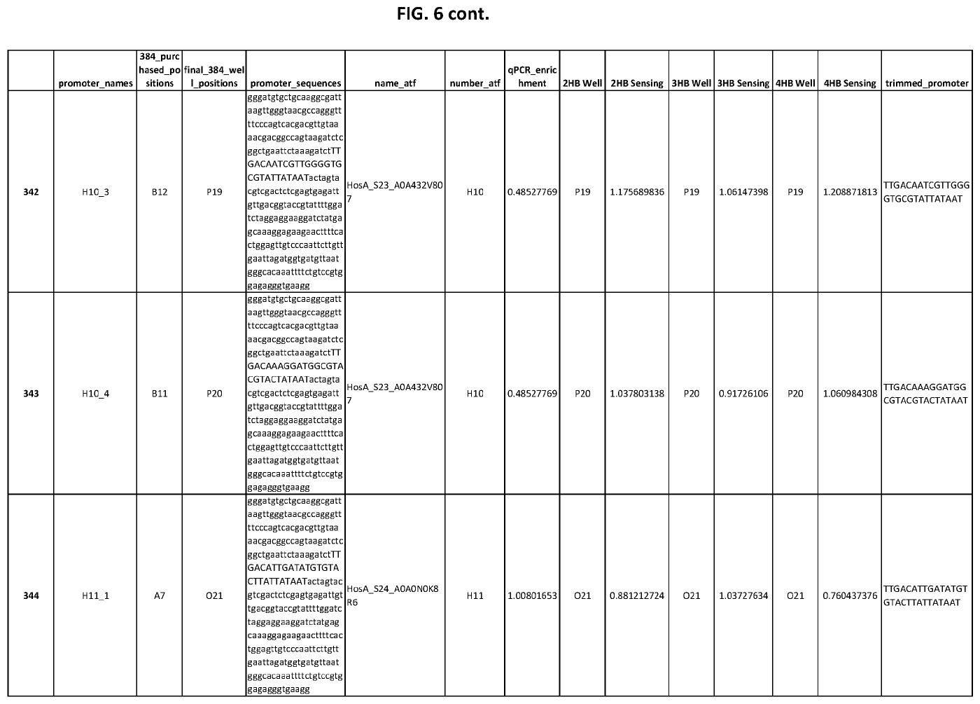

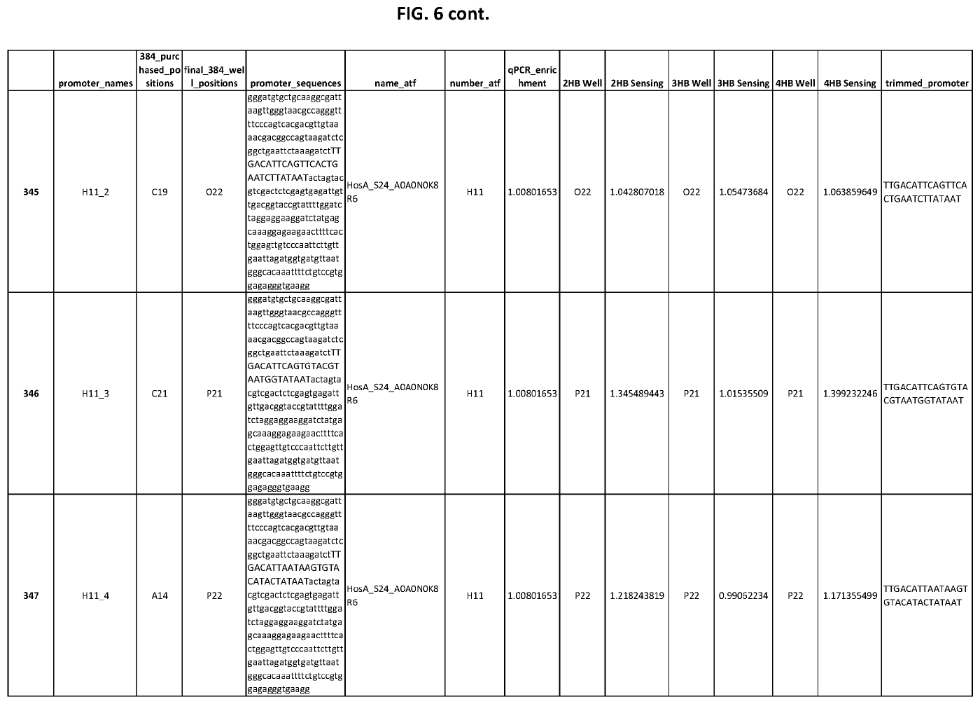

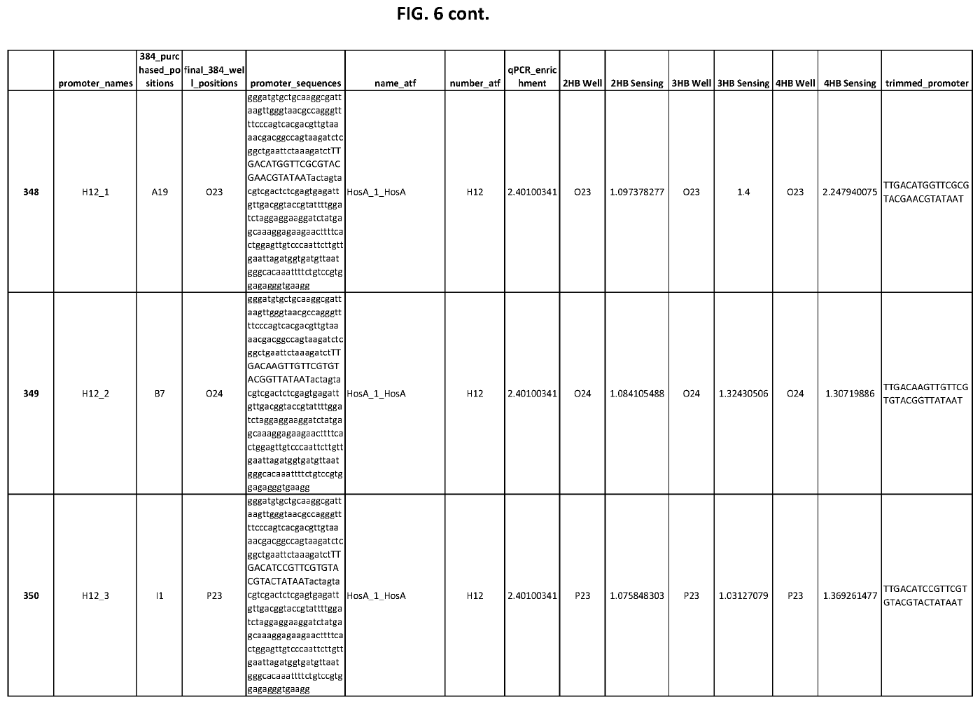

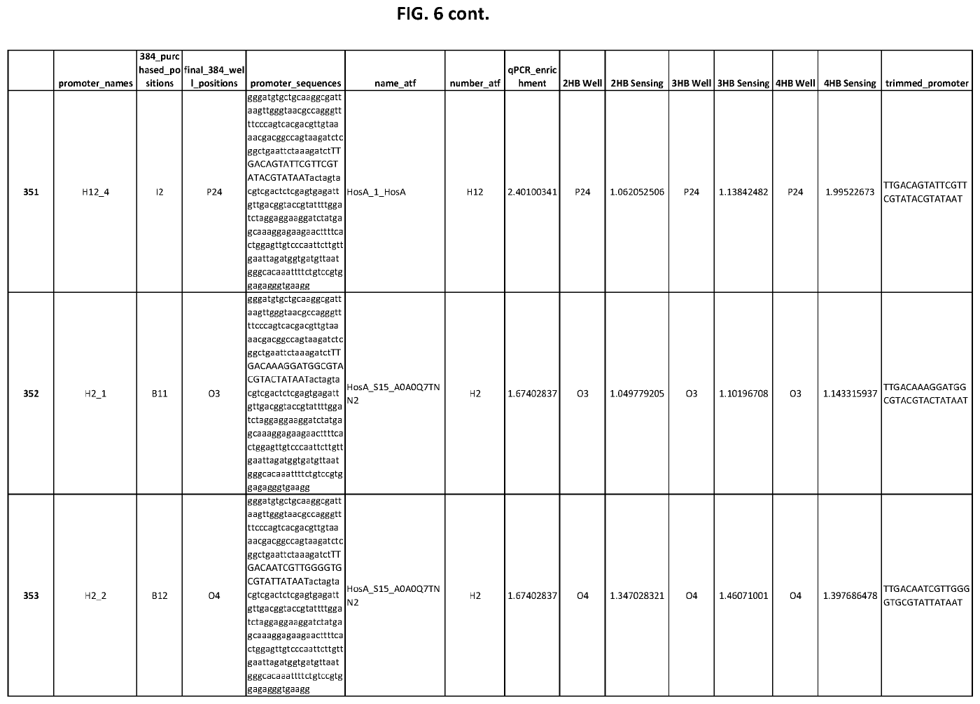

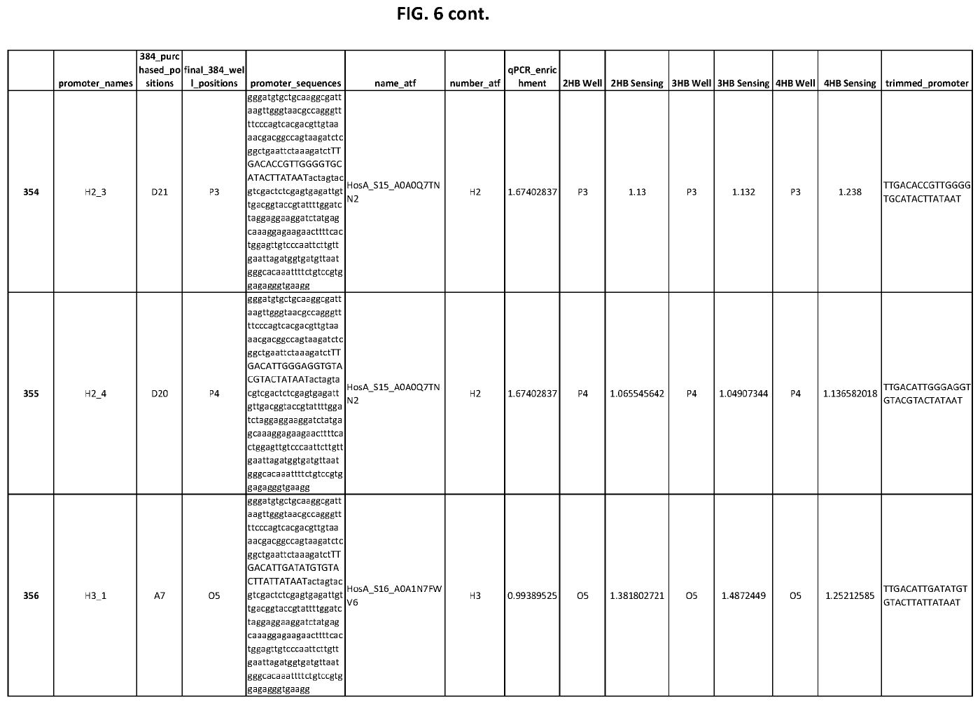

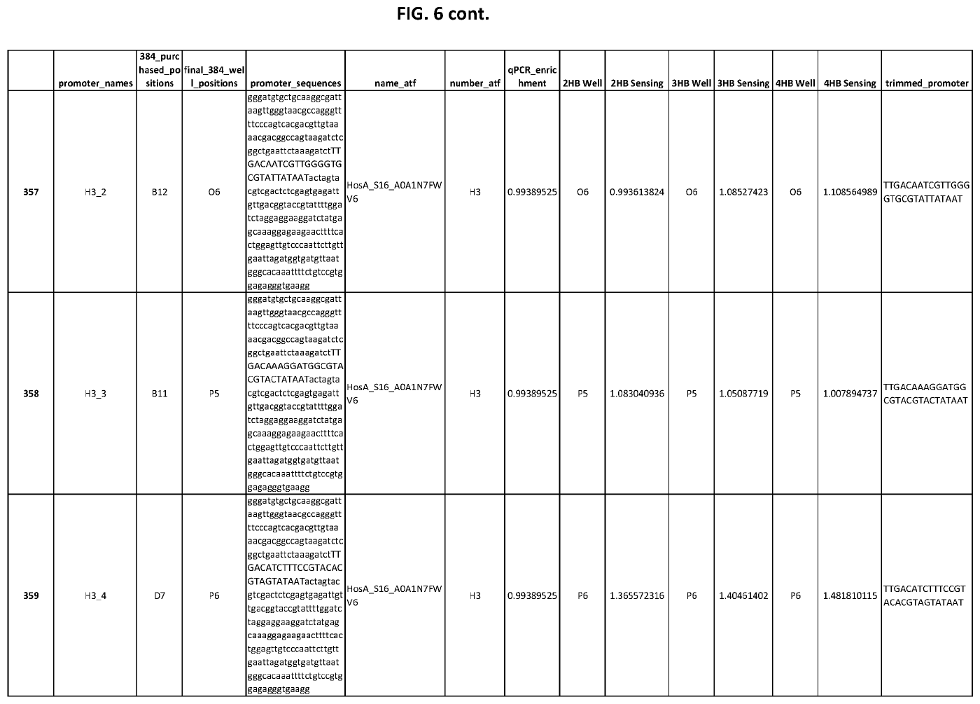

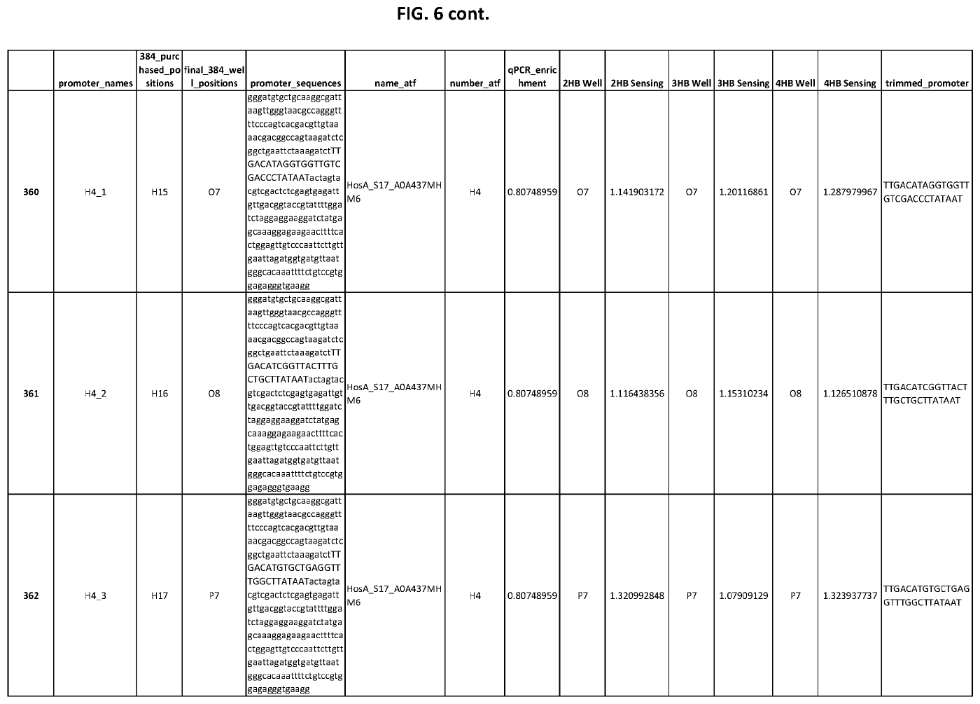

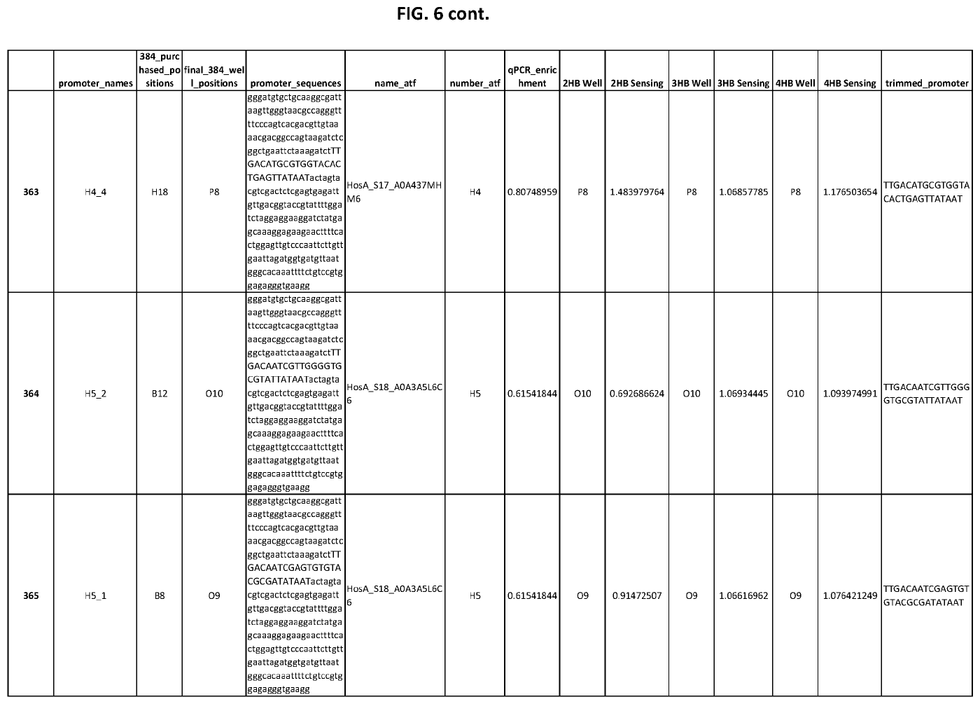

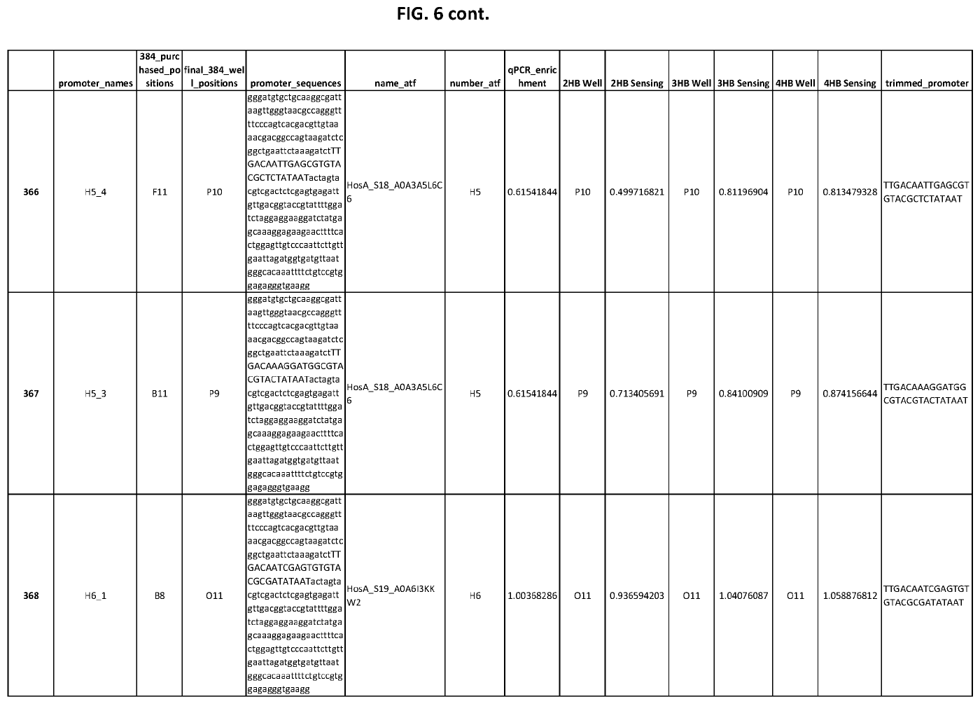

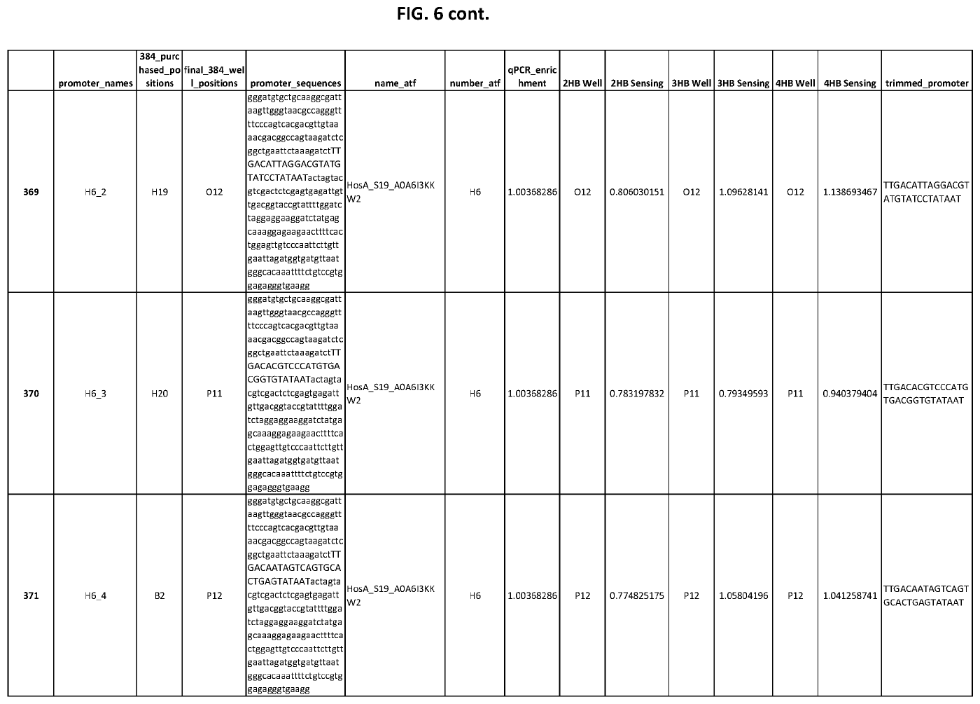

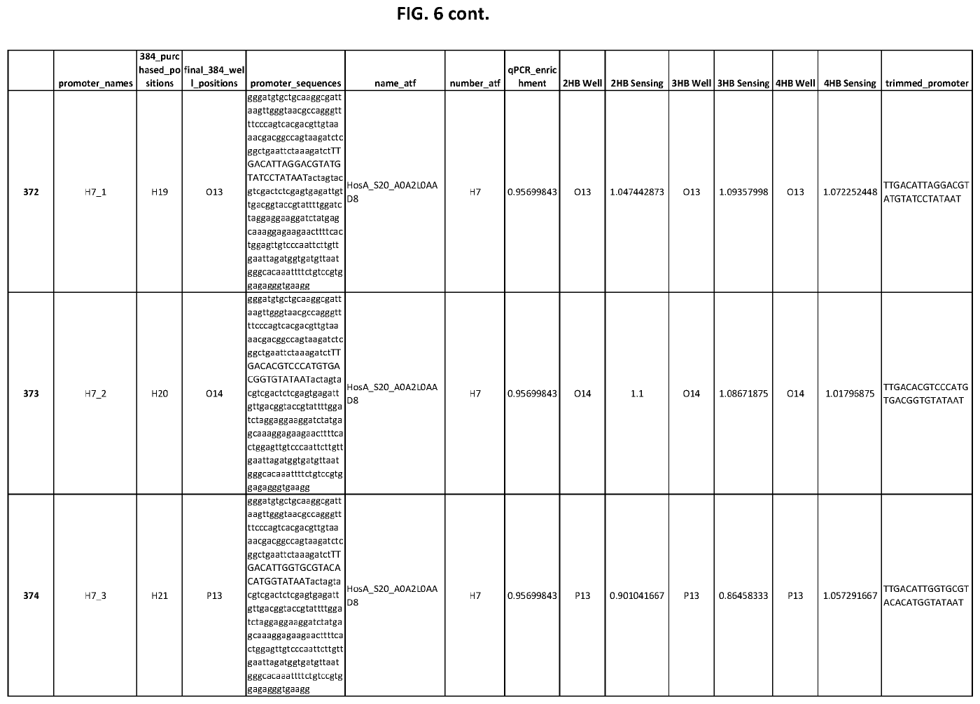

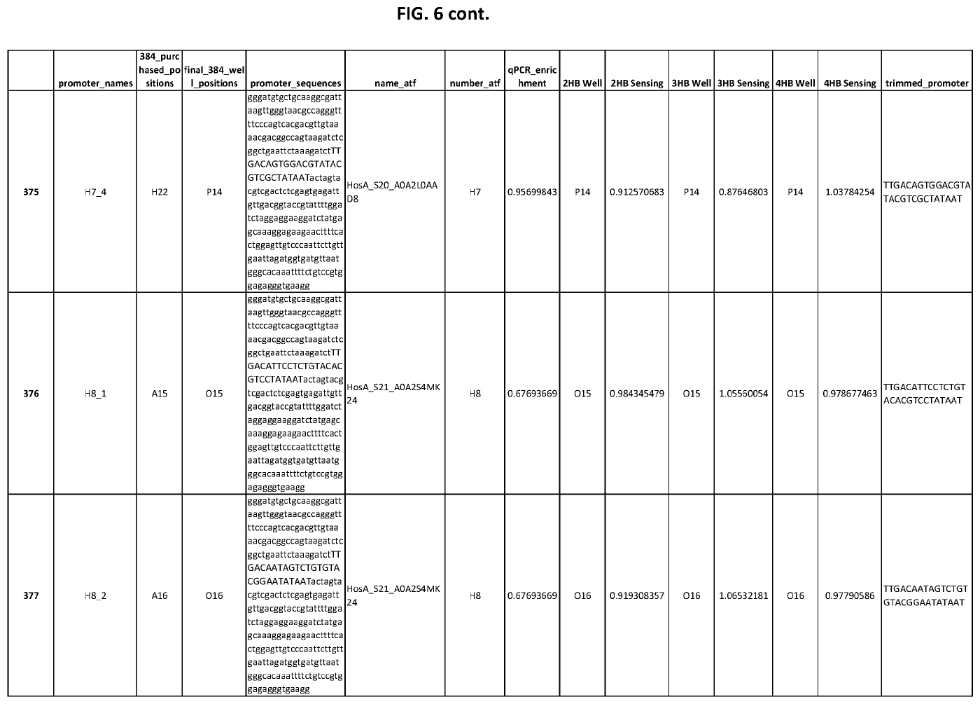

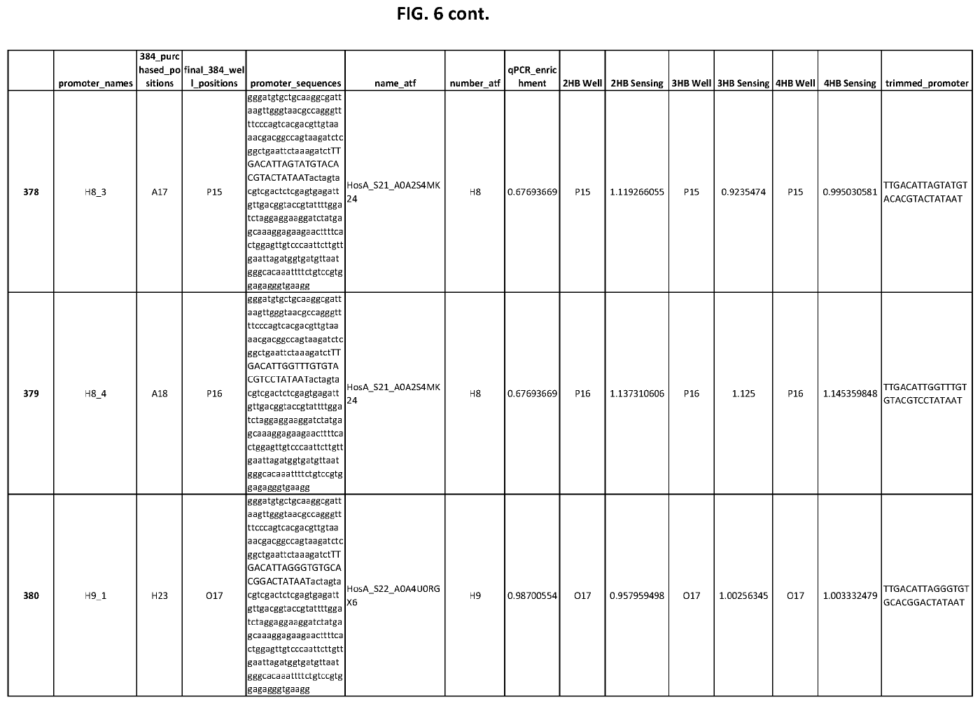

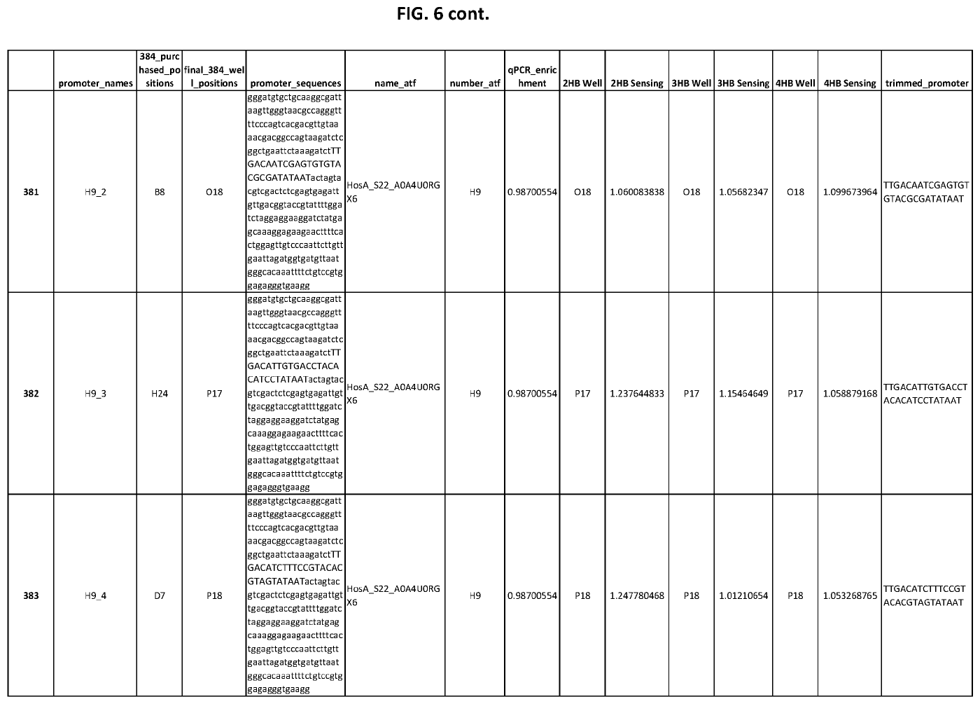

In various aspects, cell-free biosensors, kits, or systems are disclosed that can utilize a novel nucleic acid binding sequence identified using the above-described methods. In various aspects, the novel nucleic acid binding sequences can comprise a polynucleotide sequence that is at least 85%, at least 90%, at least 92%, at least 95%, at least 97%, or at least 99% identical to one or more of SEQ ID NOs: 1, 2, 4-32. In various aspects, the novel nucleic acid binding sequences can comprise or consist of a polynucleotide sequence of one or more of SEQ ID NOs: 1, 2, 4-32. In certain aspects, the nucleic acid binding sequences can comprise a polynucleotide that is at least 85%, at least 90%, at least 92%, at least 95%, at least 97%, or at least 99% identical to one or more of the operator sequences listed in FIG. 5 or 6. In various aspects, the novel nucleic acid binding sequences can comprise or consist of a polynucleotide sequence of one or more of the operator sequences listed in FIG. 5 or 6.

In various aspects, the nucleic acid binding sequences can be present in a reporter construct for use in a sensing assay and/or for use as biosensor. In various aspects, any suitable reporter construct can be utilized and a particular construct can be chosen for a particular purpose.

In various aspects, the cell-free biosensor, kits, and/or systems can include other components in addition to a polynucleotide comprising one or more of the novel nucleic acid binding sequences. In certain aspects, the cell-free biosensor, kits, and/or systems can include components for cell-free protein synthesis, e.g., such as one or more of the CFPS components described above. In various aspects, the cell-free biosensor, kits, and/or systems can include an aTF or a construct for expressing an aTF. In certain aspects, the aTF can be any aTF as mentioned above. In various aspects, the aTF can include one or more of CadR, HosA, Arac, ArsR, SriR/GutR, NanR, AllR, GylR3, TraR, BenM, MhqR, EmrR, Sav_4189, TcaR, BmrR, Betl, CamR, RamR, TetR, TtgR, and MphR. In various aspects, a construct for expressing an aTF can comprise a polynucleotide sequence that is at least 85%, at least 90%, at least 92%, at least 95%, at least 97%, at least 99%, or 100% identical to one or more of the aTF polynucleotide sequences listed in FIG. 5. In various aspects, the cell-free biosensor, kits, and/or systems can include a ligand for the aTF. In various aspects, cell-free biosensor, kits, and/or systems can include one or more reagents, buffers, or the like.

### EXAMPLES

The following Examples are illustrative and should not be interpreted to limit the scope of the claimed subject matter.

### Materials and Methods

Plasmids and DNA. See FIG. 5.

Extract preparation. See Silverman et al. ACS Synth. Biol. 2019, 8, 403-414.

Cell-free protein synthesis (CFPS). Each CFPS reaction contained the following components: 2-10 mM magnesium glutamate (optimized for each extract prep); 10 mM ammonium glutamate; 130 mM potassium glutamate; 1.2 mM ATP; 0.850 mM each of GTP, UTP, and CTP; 0.034 mg/mL folinic acid; 0.171 mg/mL yeast tRNA; 2 mM amino acids; 30 mM PEP; 0.33 mM NAD; 0.27 mM CoA; 4 mM oxalic acid; 1 mM putrescine; 1.5 mM spermidine; 57 mM HEPES; 30% CFE extract by volume; plasmid DNA was added at 13.3ng/μL or PCR reaction products was added at 13.3% of the final CFPS volume. For PCR templates, GamS (NEB) was supplied according to NEB protocols.

Linear DNA template preparation. To prepare linear templates, double stranded DNA fragments were purchased from IDT see excel file, “DNA”. Operators were gibson-assembled into the linear piece of DNA pSRF_sfGFP_backbone in front of a sfGFP gene and aTFs were gibson-assembled into the linear piece of DNA pJL1_backbone. The product of the standard gibson reaction was diluted 10-12.5× and amplified by Q5 PCR (NEB) running the following cycles from Table 1 below.

TABLE 1
PCR cycle

| Step | Celsius | Time | |
|---|---|---|---|
| 1. Initial denature | 98 | 30 | seconds |
| 2. Denature | 98 | 10 | seconds |
| 3. Anneal | 53 | 20 | seconds |
| 4. Elongate | 72 | 75 | seconds |
| 5. Final elongate | 72 | 300 | seconds |

All PCRs cycled from steps 2-4 30 times. Operator templates were amplified with primers 3 & 24 and aTF templates were amplified with primers 25 & 26. The quantity of PCR product was quantified with the QuantiFluor® dsDNA System (promega) when needed, see “Sensing assays” below.

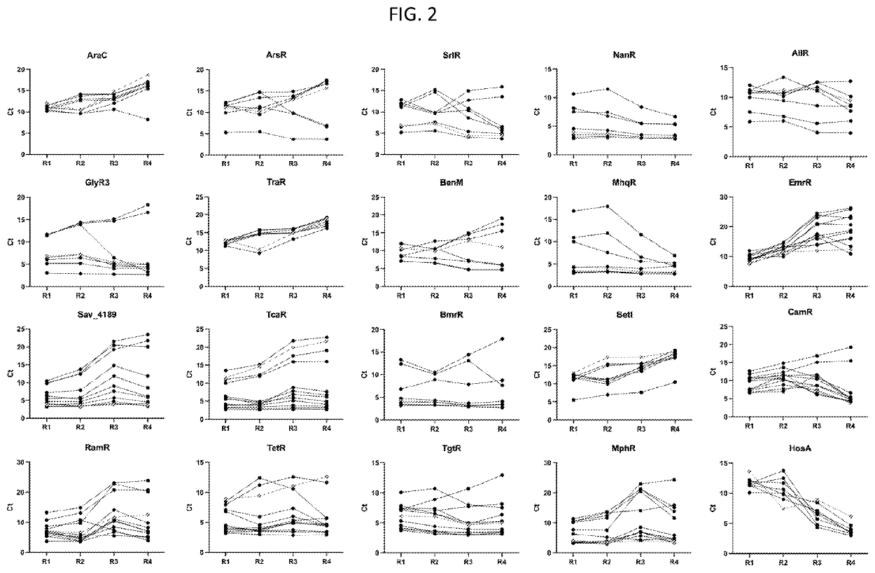

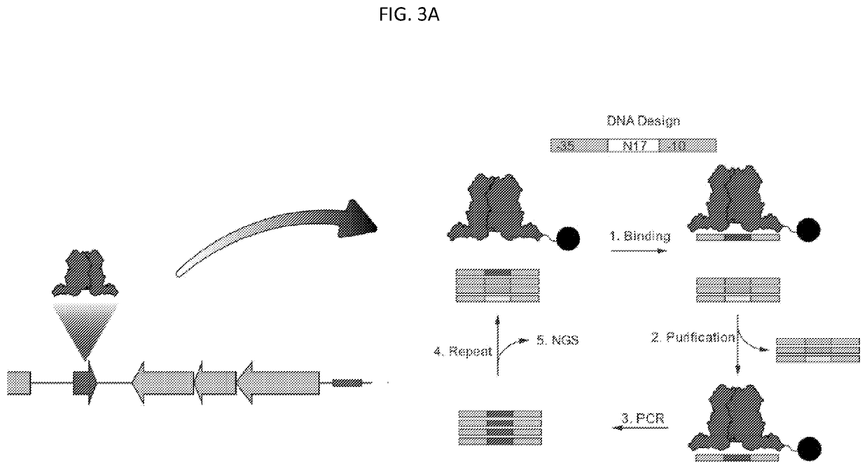

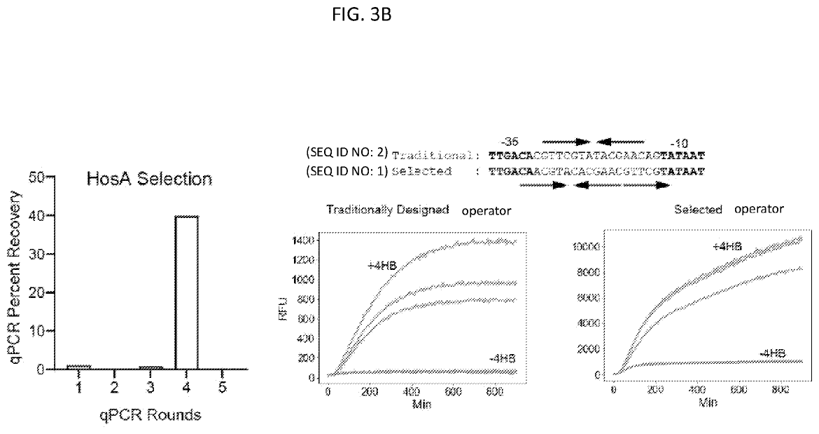

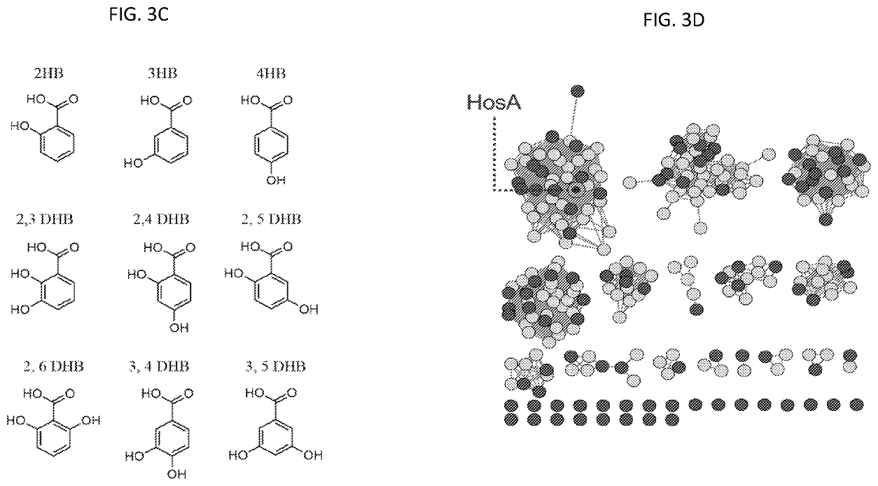

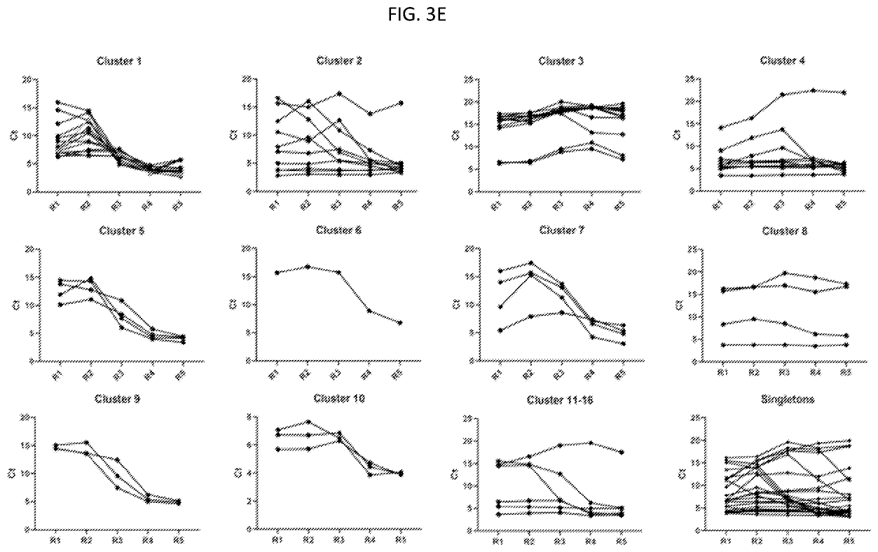

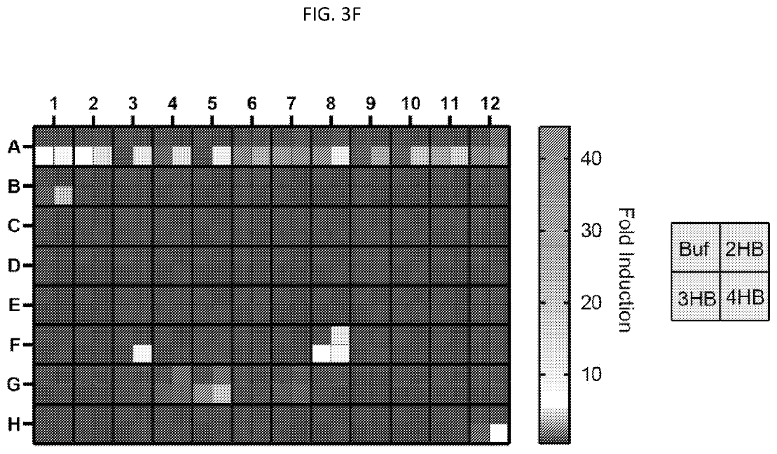

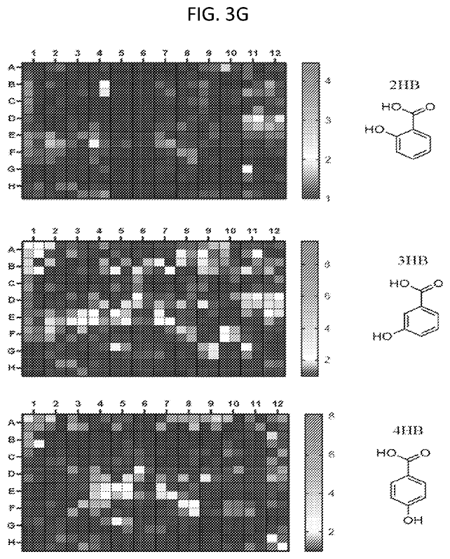

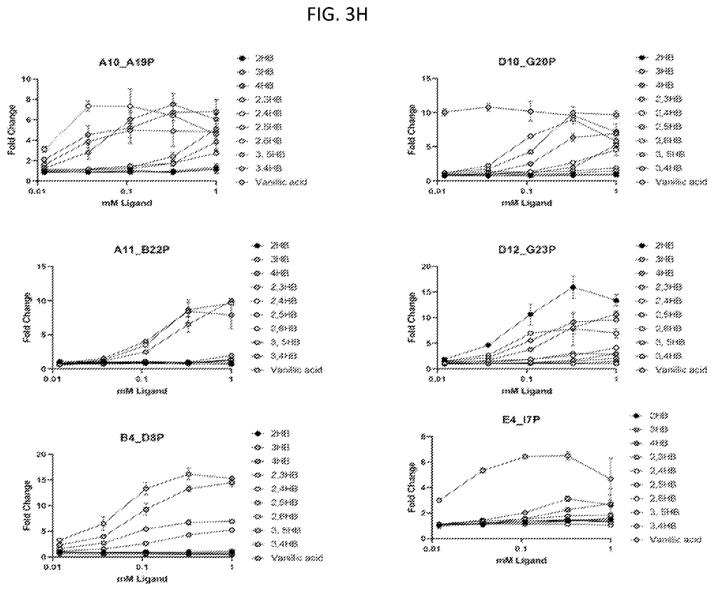

Selection method. Cell-free protein synthesis (CFPS) reactions were prepared normally with the addition of GamS to protect linear templates and 100 μM biotin to allow for biotinylation of the AviTag by endogenous BirA within the cell-free extract. The translation volumes were 45-50 μL per aTF. After overnight incubation at 30 C, CFPS reactions were supplemented with an equal volume of wash buffer (50 mM Tris pH 7.5, 150 mM NaCl, and 0.05% tween, TBS-T) along with 30 μL of TBS-T equilibrated Dynabeads™ His-Tag Isolation and Pulldown (Thermo Fisher Scientific) and rotated at 4C for 30 mins. After washing 3× with 100 μL of TBS-T, protein was eluted with 50 μL of TBS-T +250 mM imidazole pH 8.0 with rotation for 30 min at 4 C. The supernatant was recovered and the purified proteins were then added to Dynabeads™ M-280 Streptavidin beads (Thermo Fisher Scientific) for immobilization. Streptavidin beads were added to the purified proteins at 5 μL TBS-T equilibrated beads per selection round, i.e. for 4 rounds of selection, then 20 μL of streptavidin beads was added. The proteins were immobilized on streptavidin beads at 4 C with rotation for 15 mins before excess protein was removed by washing the beads 3× with 100 μL of TBS-T. To begin the first round of selection, to 5 μL of aTF-immobilized beads was added 150 ng of operator DNA library at a final volume of 50 μL. Capture of DNA was performed at 4 C with rotation for 15 min, before non-binding DNA was removed by washing the beads 3× with 100 μL of TBS-T. After the final wash, TBS-T was removed and the beads were resuspended in 50 μL of PCR buffer (10 mM Tris-HCl pH 8.5, 50 mM KCl, 0.1% Triton X-100). The captured DNA was then released by heating at 95 C for 5 min and the supernatant was collected. The amount of recovered DNA was quantified by qPCR using the original library for a standard curve. For FIG. 7, we divided the amount of DNA recovered by the total DNA input to calculate a percent recovery. For FIGS. 2b and 3c qPCR threshold values were plotted per round to monitor enrichment. Recovered DNA was then amplified by PCR and only 2 μL of the product was carried into the next round of selection. For each experiment, 4-5 rounds of selection was performed. After the final round, the amplified DNA fragments were analyzed by next generation sequencing (Genewiz, AmpliconEZ). For each selection, the total number of DNA molecules was counted and each individual operator sequence was counted. The top enriched operators were chosen for further characterization. These were purchased as double stranded DNA pieces (eblocks) from IDT and gibson-assembled with pSRF_sfGFP_backbone for sensing assays.

Sensing assays. For sensing assays, CFPS was prepared as normal in tubes, 96-well plates, or 384-well plates with two DNA templates; the aTF DNA (linear or plasmid) was added at 1 nM and the operator-sensor DNA was added at 5-20 nM. In most cases the optimal operator-sensor construct was 10 nM. For FIG. 6 (pdf) the final DNA amounts were 9 ng/μL operator-sensor linear template and 1 ng/μL aTF linear template. Ligand was added to the CFPS reactions from 0-1 mM. The amount of sfGFP synthesized was quantified using a fluorescent plate reader (excitation: 485, emission: 528). Using a standard curve, the amount of sfGFP produced was normalized to a known FITC concentration. Fold change was calculated by dividing the amount of sfGFP produced in the presence of ligand by the amount of sfGFP produced in the absence of ligand.

Bioinformatics. The sequence for HosA was obtained (Roy & Ranjan, 2016) and used as a BLAST seed on UniProt. The top 250 most similar sequences were downloaded and visualized by sequence similarity network.

### Example 1: Design and Application of a Cell-Free Operator Selection Assay

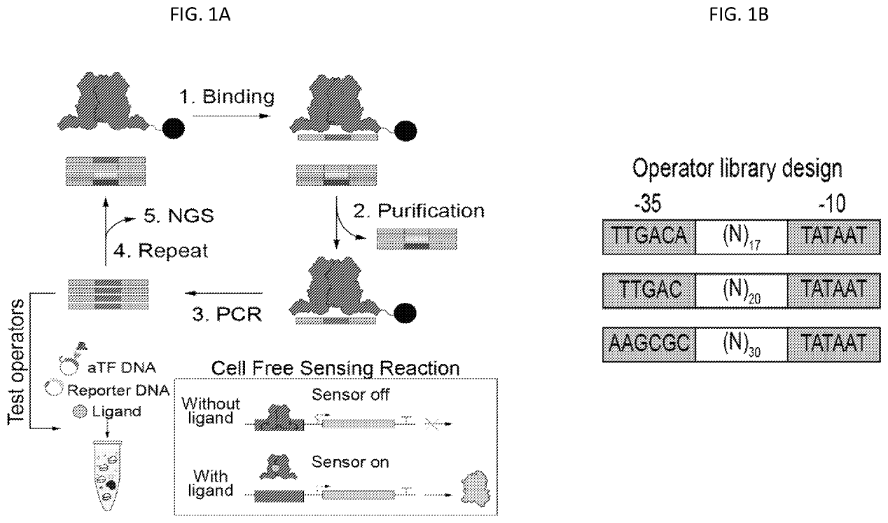

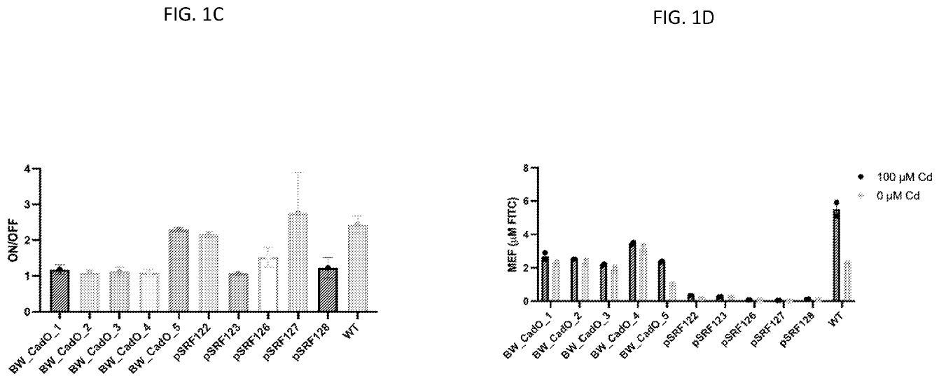

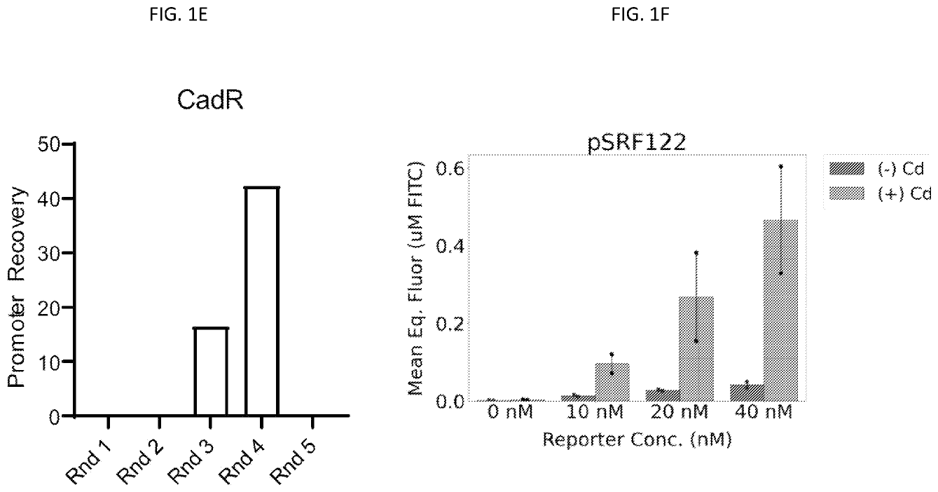

Allosteric transcription factors (aTFs) bind DNA and regulate gene transcription. Understanding the mechanism of ligand recognition and DNA interaction that stimulates RNA synthesis requires much study and engineering to turn these biological regulators into biosensors. While the mechanism for aTF gene activation can be complicated and vary widely, all aTFs bind DNA and their affinity or interaction with DNA changes upon ligand binding. Taking advantage of this characteristic, we've designed a simple SELEX assay that selects the DNA binding site of an aTF within the context of the strong, constitutive E. coli promoter J23119, FIGS. 1A and 1B. This results in a very simple operator. Namely, when the aTF is present it binds the promoter and precludes RNA Polymerase (RNAP) from initiating RNA synthesis. When the cognate ligand is supplied and binds the aTF we expect an allosteric shift in the protein to weaken the interaction of the aTF with DNA and allow RNAP to bind and synthesize RNA leading to robust expression of sfGFP, FIG. 1A. To test this idea, we chose to develop assay conditions with CadR, a cadmium responsive transcription factor, as our model. After multiple rounds of selection, winning operators were cloned into a sfGFP expressing plasmid and tested in a cell-free biosensing reaction.

Three DNA operator libraries of the sigma70 promoter were designed with 17, 20or 30 randomized nucleotides (N17, N20, N30) upstream of a fixed −10 region. For N17, −35 was also fixed, N20 contained a partial −35 and N30 had no fixed −35 (FIG. 1B).

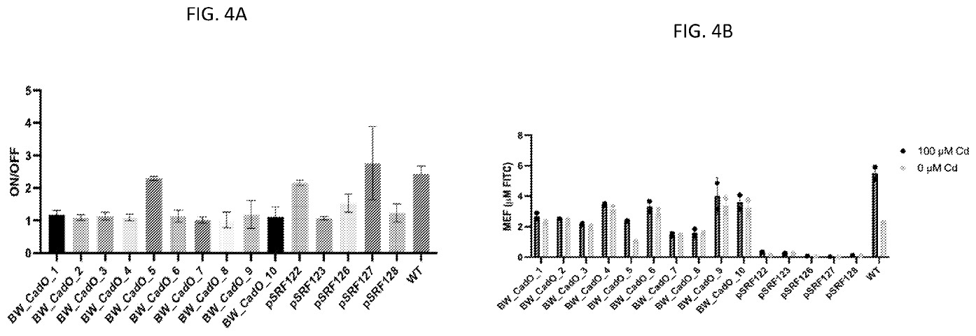

After four rounds of selection with libraries N17, N20, and N30, next generation sequencing (NGS) analysis revealed enriched operators for CadR. These operators were cloned into our sfGFP expressing vector and tested for cadmium sensing. A previously developed operator for CadR was used as a WT control. Although the ON signal varied widely, each selection produced a viable cadmium sensor with a similar ON/OFF ratio to the WT construct (see FIG. 1C and 1D and FIG. 4A and 4B).

Operators with the highest ON/OFF ratio from each library are shown in the below Table 2. Little similarity is seen between each promoter except the presence of an “AGGGTT” motif.

TABLE 2
Operator winners from each library

| | SEQ | | |
|---|---|---|---|
| | ID | | |
| Operator | NO: | Sequence | Library |
| BW_CadO_ | 8 | TTGACATTGTAGCTACTTCAGGGTATA | N17 |
| 5 | | AT | |
| pSRF122 | 15 | TTGACGTTAGATGTAACCTGAGGGTTA | N20 |
| | | TAAT | |
| pSRF127 | 20 | AAGCGCATCTTGGCTAACCCTGAAGCT | N30 |
| | | ACTTCACTGTATAAT | |
| WT | 3 | TTGACTCTGTAGTTGCTACAGGGTGTA | |
| | | TAAT | |

FIG. 1E shows the relative amounts of operator recovery per round of qPCR. FIG. 1F shows the relative amounts of reporter activation in the presences of varying amounts of Cadmium for the pCRF122 operator. Table 3 below lists the CadR operator variants tested in this Example, along with the wild type (WT) operator sequence.

TABLE 3
Operator sequences

| | SEQ | CadO | | |
|---|---|---|---|---|
| Name | NO: | Variant | Operator Sequence | Library |
| BW_CadO_1 | 4 | 1 | TTGACATCAGGGTTACTACAATGTATAAT | N17 |
| BW_CadO_2 | 5 | 2 | TTGACATCAGGGTTGGTGTAGGGTATAAT | N17 |
| BW_CadO_3 | 6 | 3 | TTGACATCAGGGTTACTTAAGGGTATAAT | N17 |
| BW_CadO_4 | 7 | 4 | TTGACATCAGGGTTGGTGGAGGGTATAAT | N17 |
| BW_CadO_5 | 8 | 5 | TTGACATTGTAGCTACTTCAGGGTATAAT | N17 |
| BW_CadO_6 | 9 | 6 | TTGACATTAGGGTTAGTTTAGACTATAAT | N17 |
| BW_CadO_7 | 10 | 7 | TTGACATCATTGTTACTTTAGGGTATAAT | N17 |
| BW_CadO_8 | 11 | 8 | TTGACATCAGGGTCGGTATAGGGTATAAT | N17 |
| BW_CadO_9 | 12 | 9 | TTGACATGAATGTTGGTACAGGGTATAAT | N17 |
| BW_CadO_10 | 13 | 10 | TTGACATGAGGGTTGCTTCAGAGTATAAT | N17 |
| pSRF121 | 14 | 11 | TTGACGTTAGGGTTAACGTTCAAAGTATAAT | N20 |
| pSRF122 | 15 | 12 | TTGACGTTAGATGTAACCTGAGGGTTATAAT | N20 |
| pSRF123 | 16 | 13 | TTGACCCTTACTTGAACGTTACTGTTATAAT | N20 |
| pSRF124 | 17 | 14 | TTGACTCTAGCTTTAGGGTATTGACTATAAT | N20 |
| pSRF125 | 18 | 15 | TTGACTCTAAGGTTATACTTGGCGCTATAAT | N20 |
| pSRF126 | 19 | 16 | AAGCGCTCAAATGAAGGTTTGAGTGAAGGTAAGGGTTATAAT | N30 |
| pSRF127 | 20 | 17 | AAGCGCATCTTGGCTAACCCTGAAGCTACTTCACTGTATAAT | N30 |
| pSRF128 | 21 | 18 | AAGCGCTTAACTATGACGTTAAATTGAACTATAGGGTATAAT | N30 |
| pSRF129 | 22 | 19 | AAGCGCATGTAGTTGACCTTAACGTGAGGGTTATAGTATAAT | N30 |
| pSRF130 | 23 | 20 | AAGCGCGAACCTTGAGGTTGAATTGAAGGTTAGGGTTATAAT | N30 |
| WT | 3 | | TTGACTCTGTAGTTGCTACAGGGTGTATAAT | |

### Example 2: aTF SELEX Works across Known aTF Classes

We tested the generality of this SELEX assay described in Example 1 to develop cell-free biosensors for several other bacterial transcription factors with known ligands. Importantly, we chose aTFs that sense a wide range of molecules and include some of the most studied bacterial transcription factor classes, such as: AraC, ArsR, DeoR, GntR, IclR, LacI, LuxR, LysR, MarR, MerR, TetR, XylR. (See Table 4 below)

TABLE 4
aTFs of varying classes used in SELEX

| aTF | Ligand | Class | Type |
|---|---|---|---|
| AraC | L-arabinose | AraC | Co-activator |
| ArsR | Arsenic | ArsR | Repressor |
| SrlR/GutR | Glucitol | DeoR | Repressor |
| NanR | N-Acetylneuraminate | GntR | Apo-repressor |
| AIIR | Glyoxylate | IclR | Apo-repressor |
| GylR3 | Laminaribiose | Lacl | Apo-Repressor |
| TraR | 3-oxooctanoyl-homoserine | LuxR | Co-activator |
| BenM | Cis cis muconic acid | LysR | Co-activator |
| MhqR | Methylhydroquinone | MarR | Apo-Repressor |
| EmrR | Dopamine | MarR | Apo-Activator |
| Sav_4189 | Hygromycin B | MarR | Apo-Repressor |
| TcaR | Teicoplanin | MarR | Repressor |
| BmrR | Rhodamine and | MerR | Activator |
| | tetraphenylphosphonium | | |
| | (TPP) | | |
| Betl | Choline | TetR | Repressor |
| CamR | Camphor | TetR | Apo-Repressor |
| RamR | Berberine | TetR | Apo-Repressor |
| TetR | Tetracycline | TetR | Apo-Repressor |
| TtgR | Phloretin (Antibiotics | TetR | Repressor |
| | and plant secondary | | |
| | metabolites) | | |
| MphR | Erythromycin | XylR/DmpR | Repressor |

Adjusting the selection protocol for 96-well plates, we selected 8 to 12 homologs of each aTF from Table 4 above (and for HosA) and subjected them to our SELEX operator selection assay described in Example 1. Of note, we chose to move forward only with the N17 library, to serve as our general operator library.

The selection for each aTF was monitored by qPCR. The results of qPCR threshold values from 4 rounds of selection were plotted in FIG. 2. As the selection rounds increase, if the library is becoming enriched for binders, then more DNA should be recovered per round and is indicated by a dropping Ct value, FIG. 2. Based on this qPCR data roughly half of the tested transcription factors had at least one homolog that trended towards an enriched library. Red line indicates initial aTF used in BLAST to find homologs.

A heatmap of each aTF and the activity of four selected operators for sensing each aTF's known ligand will be made.

### Example 3: Discover of Biosensors for New Ligands

In our pan aTF selection of Example 2, HosA stood out as an interesting test case to see if we could discover biosensors for new ligands. HosA has been shown to selectively sense 4HB (Roy & Ranjan, 2016), but other similar looking hydroxy acids are found in nature (see FIG. 3C). We performed a blast search with HosA as the seed and collected 250 homologs from UniProt. The sequence diversity was analyzed using a sequence similarity network (SSN) and visualized using Cytoscape. The visualization of the SSN is shown in FIG. 3D with 16 clusters and a singletons. Selecting a few homologs from each cluster, we scanned the entire diversity of the SSN map in our operator selection assay using the J23119 promoter (detailed in FIG. 3A), following the library enrichment by qPCR (see FIG. 3E). Many of the clusters showed trends for an enriched library of operators. FIG. 3B shows the results of a SELEX selection with HosA and validation of a selected promoter.

The top 4 enriched operators were selected for each homolog and tested for sensing 2HB, 3HB, and 4HB. All hits from cluster 1 turned on strongly in the presence of 4HB. Additionally, cluster 7 turned on lightly. In addition, we found putative sensors for 2HB and 3HB (see FIG. 3F). FIG. 3F shows the results of the 384 selected aTF/operator pairs (96 HosA homologs with each of the 4 selected promoters) that were tested for sensing 1 mM 2HB, 3HB, and 4HB. Blue indicates OFF while white and red indicate and ON signal. A1-H12 represent distinct aTFs and each small square is a selected promoter from the library enrichment mentioned above. FIG. 3G shows the sensing reactions or assays with the HosA promoter with 96 HosA homologs. A1-H12 represent distinct aTFs and each small square is buffer or a hydroxybenzoic acid, 2HB, 3HB, or 4HB.

A few cell-free biosensors showing selectivity for 2HB, 3HB, or 4HB were chosen for more rigorous experimentation. Interestingly, we found potent biosensors for each, and even found sensors that were highly sensitive for vanillic acid and protocatechuate (see FIG. 3H). Table 5 below lists representative selected promoters per HosA homolog cluster and the putuative molecules they sense. Table 5 summarizes the sensing data from FIG. 3G and in the Table of FIG. 6, binning the aTFs by cluster and describing which molecules they sense. Additionally, a highly active operator was chosen as a cluster representative.

TABLE 5
Representative promoters per HosA homolog cluster

| | aTF | | qPCR | | |
|---|---|---|---|---|---|
| Cluster | Names | Molecules | Enrichment | Representative Promoter | SEQ ID NO: |
| 1 | A1-B1 | 4HB | Yes | TTGACACGTTCGTATACGAACAGTATAAT | 2 |
| 2 | B2- | 3HB | Yes | TTGACATTGATATGTGTACTTATTATAAT | 24 |
| | B11 | | | | |
| 3 | B12- | — | No | — | |
| | C11 | | | | |
| 4 | C12- | 3HB,4HB | Yes | TTGACAATAGTCAGTGCACTGAGTATAAT | 25 |
| | D10 | | | | |
| 5 | D11- | 2HB,3HB | Yes | TTGACATTGATATGTGTACTTATTATAAT | 26 |
| | E2 | | | | |
| 6 | E3 | 3HB | Yes | TTGACACATATGATATGTATACGTATAAT | 27 |
| 7 | E4-E7 | 3HB,4HB | Yes | TTGACATTCAGTTCACTGAATCTTATAAT | 28 |
| 8 | E8- | — | No | — | |
| | E11 | | | | |
| 9 | E12- | 3HB? | Yes | TTGACAATCGAGCGTGTACGCTCTATAAT | 29 |
| | F2 | | | | |
| 10 | F3-F5 | — | No | — | |
| 11 | F6 | ? | Yes | TTGACATTCTTCAGTGTACTGAATATAAT | 30 |
| 12 | F7 | — | No | — | |
| 13 | F8 | ? | Yes | TTGACATTGCTTCGTATACGAACTATAAT | 31 |
| 14 | F9 | — | No | — | |
| 15 | F10 | 3HB | No | TTGACAGCTGGATCATTGGTGTGTATAAT | 32 |
| 16 | F11 | — | No | - | |

We have developed an assay that quickly onboards new aTFs for biosensing reactions in cell-free systems. We have shown the versatility of the assay by selecting operators for repressors and activators and developing hundreds of new aTFs that sense high value molecules. These sensors are ready for point of care detection, high throughput monitoring of chemical reactions, and are ready to be explored in cellular environments to develop new genetic circuits. With the presented protocols we can now think about biosensing in a molecule-centric manner.

The disclosure herein may also be described in accordance with the below numbered clauses.

Clause 1. A method for generating novel nucleic acid sequences that bind to an allosteric transcription factor comprising: (i) providing a library of partially randomized nucleic acid sequences, an allosteric transcription factor (aTF), and a ligand for the aTF; (ii) contacting the library of partially randomized nucleic acid sequences to the aTF in the presence of the ligand to produce one or more nucleic acid-aTF complexes and non-bound nucleic acid sequences; (iii) partitioning the nucleic acid-aTF complexes away from the non-bound nucleic acid sequences; (iv) purifying the nucleic acid sequences from the nucleic acid-aTF complexes to generate purified nucleic acid sequences; and (v) amplifying the purified nucleic acid sequences to generate amplified purified nucleic acid sequences.

Clause 2. The method of clause 1, further comprising: repeating the method steps (ii)-(iv) using the amplified purified nucleic acid sequences generated by step (v), and optionally repeating step (v) to provide enriched amplified purified nucleic acid sequences.

Clause 3. The method of clause 1 or 2, wherein the nucleic acid sequences are DNA sequences.

Clause 4. The method of any one of clauses 1-3, wherein the allosteric transcription factor is CadR, HosA, Arac, ArsR, SriR/GutR, NanR, AllR, GylR3, TraR, BenM, MhqR, EmrR, Sav_4189, TcaR, BmrR, Betl, CamR, RamR, TetR, TtgR, or MphR.

Clause 5. The method of clause 4, wherein the allosteric transcription factor is CadR or HosA.

Clause 6. The method of any one of clauses 1-5, wherein the allosteric transcription factor comprises an affinity tag or motif.

Clause 7. The method of any one of clauses 1-6, wherein the library of partially randomized nucleic acid sequences comprises nucleic acid sequences about 98% similar, about 95% similar, about 90% similar, about 85% similar, about 80% similar, about 75% similar, or about 70% similar to a native operator sequence that is recognized by the aTF.

Clause 8. The method of any one of clauses 1-7, wherein the library of nucleic acid sequences comprises nucleic acid sequences that are at least 70% identical, at least 80% identical, at least 85% identical, or at least 90% identical to SEQ ID NO: 3.

Clause 9. The method of clause 2, further comprising: performing a sensing assay that includes the use of one or more of the enriched amplified purified nucleic acid sequences present in a reporter gene construct.

Clause 10. A system for generating novel nucleic acid sequences that bind to an allosteric transcription factor (aTF) comprising: a library of partially randomized nucleic acid sequences that comprise nucleic acid sequences that are similar to a native nucleic acid sequence that binds to an aTF but differ in at least one nucleic acid residue from the native nucleic acid sequence, an aTF, and a ligand to the aTF.

Clause 11. The system of clause 10, wherein the library of partially randomized nucleic acid sequences comprises nucleic acid sequences about 98% similar, about 95% similar, about 90% similar, about 85% similar, about 80% similar, about 75% similar, or about 70% similar to a native operator sequence that is recognized by the aTF.

Clause 12. The system of clause 10 or 11, wherein the library of partially randomized nucleic acid sequences comprises nucleic acid sequences that are at least 70% identical, at least 80% identical, at least 85% identical, or at least 90% identical to SEQ ID NO: 3.

Clause 13. The system of any one of clauses 10-12, wherein the system further comprises: a polymerase.

Clause 14. The system of any one of clauses 10-13, wherein the allosteric transcription factor is CadR, HosA, Arac, ArsR, SriR/GutR, NanR, AllR, GylR3, TraR, BenM, MhqR, EmrR, Sav_4189, TcaR, BmrR, Betl, CamR, RamR, TetR, TtgR, or MphR.

Clause 15. The system of clause 14, wherein the allosteric transcription factor is CadR or HosA.

Clause 16. The system of any one of clauses 10-15, wherein the allosteric transcription factor comprises an affinity tag or motif.

Clause 17. The system of any one of clauses 10-16, wherein the nucleic acid sequences are DNA sequences and/or wherein the allosteric transcription factor is HosA or CadR.

Clause 18. A nucleic acid sequence having the polynucleotide sequence of any one of SEQ ID NOs: 1, 2, 4-32, or a polynucleotide sequence that is at least about 90% identical to any one of SEQ ID NOs: 1, 2, 4-32.

Clause 19. A cell-free biosensor, kit, method, or system comprising a novel aTF nucleic acid binding sequence identified by any of the methods or systems of clauses 1-18.

Clause 20. The cell-free biosensor, kit, method, or system of clause 19, wherein the novel aTF nucleic acid binding sequence comprises the polynucleotide sequence of any one of SEQ ID NOs: 1, 2, 4-32, or a polynucleotide sequence that is at least about 90% identical to any one of SEQ ID NOs: 1, 2, 4-32.

Clause 21. The cell-free biosensor, kit, method, or system of clause 19 or 20, further comprising an allosteric transcription factor or a polynucleotide sequence encoding for the allosteric transcription factor.

Clause 22. The cell-free biosensor, kit, method, or system of clause 21, wherein the allosteric transcription factor is CadR, HosA, Arac, ArsR, SriR/GutR, NanR, AllR, GylR3, TraR, BenM, MhqR, EmrR, Sav_4189, TcaR, BmrR, Betl, CamR, RamR, TetR, TtgR, or MphR

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A method for generating novel nucleic acid sequences that bind to an allosteric transcription factor comprising:
(i) providing a library of partially randomized nucleic acid sequences, an allosteric transcription factor (aTF), and a ligand for the aTF;
(ii) contacting the library of partially randomized nucleic acid sequences to the aTF in the presence of the ligand to produce one or more nucleic acid-aTF complexes and non-bound nucleic acid sequences;
(iii) partitioning the nucleic acid-aTF complexes away from the non-bound nucleic acid sequences;
(iv) purifying the nucleic acid sequences from the nucleic acid-aTF complexes to generate purified nucleic acid sequences; and
(v) amplifying the purified nucleic acid sequences to generate amplified purified nucleic acid sequences.

2. The method of claim 1, further comprising: repeating the method steps (ii)-(iv) using the amplified purified nucleic acid sequences generated by step (v), and optionally repeating step (v) to provide enriched amplified purified nucleic acid sequences.

3. The method of claim 1, wherein the nucleic acid sequences are DNA sequences.

4. The method of claim 1, wherein the allosteric transcription factor is CadR, HosA, Arac, ArsR, SriR/GutR, NanR, AllR, GylR3, TraR, BenM, MhqR, EmrR, Sav_4189, TcaR, BmrR, Betl, CamR, RamR, TetR, TtgR, or MphR.

5. The method of claim 4, wherein the allosteric transcription factor is CadR or HosA.

6. The method of claim 1, wherein the allosteric transcription factor comprises an affinity tag or motif.

7. The method of claim 1, wherein the library of partially randomized nucleic acid sequences comprises nucleic acid sequences about 98% similar, about 95% similar, about 90% similar, about 85% similar, about 80% similar, about 75% similar, or about 70% similar to a native operator sequence that is recognized by the aTF.

8. The method of claim 1, wherein the library of nucleic acid sequences comprises nucleic acid sequences that are at least 70% identical, at least 80% identical, at least 85% identical, or at least 90% identical to SEQ ID NO: 3.

9. The method of claim 2, further comprising: performing a sensing assay that includes the use of one or more of the enriched amplified purified nucleic acid sequences present in a reporter gene construct.

10. A system for generating novel nucleic acid sequences that bind to an allosteric transcription factor (aTF) comprising:
a library of partially randomized nucleic acid sequences that comprise nucleic acid sequences that are similar to a native nucleic acid sequence that binds to an aTF but differ in at least one nucleic acid residue from the native nucleic acid sequence, an aTF, and a ligand to the aTF.

11. The system of claim 10, wherein the library of partially randomized nucleic acid sequences comprises nucleic acid sequences about 98% similar, about 95% similar, about 90% similar, about 85% similar, about 80% similar, about 75% similar, or about 70% similar to a native operator sequence that is recognized by the aTF.

12. The system of claim 10, wherein the library of partially randomized nucleic acid sequences comprises nucleic acid sequences that are at least 70% identical, at least 80% identical, at least 85% identical, or at least 90% identical to SEQ ID NO: 3.

13. The system of claim 10, wherein the system further comprises: a polymerase.

14. The system of claim 10, wherein the allosteric transcription factor is CadR, HosA, Arac, ArsR, SriR/GutR, NanR, AllR, GylR3, TraR, BenM, MhqR, EmrR, Sav_4189, TcaR, BmrR, Betl, CamR, RamR, TetR, TtgR, or MphR.

15. The system of claim 14, wherein the allosteric transcription factor is CadR or HosA.

16. The system of claim 10, wherein the allosteric transcription factor comprises an affinity tag or motif.

17. The system of claim 10, wherein the nucleic acid sequences are DNA sequences and/or wherein the allosteric transcription factor is HosA or CadR.

18. A nucleic acid sequence having the polynucleotide sequence of any one of SEQ ID NOs: 1, 2, 4-32, or a polynucleotide sequence that is at least about 90% identical to any one of SEQ ID NOs: 1, 2, 4-32.

19. (canceled)

20. A cell-free biosensor, kit, method, or system comprising a novel aTF nucleic acid binding sequence, wherein the novel aTF nucleic acid binding sequence comprises the polynucleotide sequence of any one of the nucleic acid sequences of claim 18.

21. The cell-free biosensor, kit, method, or system of claim 20, further comprising an allosteric transcription factor or a polynucleotide sequence encoding for the allosteric transcription factor, wherein the allosteric transcription factor is CadR, HosA, Arac, ArsR, SriR/GutR, NanR, AllR, GylR3, TraR, BenM, MhqR, EmrR, Sav_4189, TcaR, BmrR, Betl, CamR, RamR, TetR, TtgR, or MphR.

22. (canceled)


SEQUENCE LISTING (32 sequences)

SEQ ID NO: 1 - DNA, 29 bp, synthetic construct
  source 1..29: mol_type = other DNA
ttgacaacgtacacgaacgttcgtataat

SEQ ID NO: 2 - DNA, 29 bp, synthetic construct
  source 1..29: mol_type = other DNA
ttgacacgttcgtatacgaacagtataat

SEQ ID NO: 3 - DNA, 31 bp, synthetic construct
  source 1..31: mol_type = other DNA
ttgactctgtagttgctacagggtgtataat

SEQ ID NO: 4 - DNA, 29 bp, synthetic construct
  source 1..29: mol_type = other DNA
ttgacatcagggttactacaatgtataat

SEQ ID NO: 5 - DNA, 29 bp, synthetic construct
  source 1..29: mol_type = other DNA
ttgacatcagggttggtgtagggtataat

SEQ ID NO: 6 - DNA, 29 bp, synthetic construct
  source 1..29: mol_type = other DNA
ttgacatcagggttacttaagggtataat

SEQ ID NO: 7 - DNA, 29 bp, synthetic construct
  source 1..29: mol_type = other DNA
ttgacatcagggttggtggagggtataat

SEQ ID NO: 8 - DNA, 29 bp, synthetic construct
  source 1..29: mol_type = other DNA
ttgacattgtagctacttcagggtataat

SEQ ID NO: 9 - DNA, 29 bp, synthetic construct
  source 1..29: mol_type = other DNA
ttgacattagggttagtttagactataat

SEQ ID NO: 10 - DNA, 29 bp, synthetic construct
  source 1..29: mol_type = other DNA
ttgacatcattgttactttagggtataat

SEQ ID NO: 11 - DNA, 29 bp, synthetic construct
  source 1..29: mol_type = other DNA
ttgacatcagggtcggtatagggtataat

SEQ ID NO: 12 - DNA, 29 bp, synthetic construct
  source 1..29: mol_type = other DNA
ttgacatgaatgttggtacagggtataat

SEQ ID NO: 13 - DNA, 29 bp, synthetic construct
  source 1..29: mol_type = other DNA
ttgacatgagggttgcttcagagtataat

SEQ ID NO: 14 - DNA, 31 bp, synthetic construct
  source 1..31: mol_type = other DNA
ttgacgttagggttaacgttcaaagtataat

SEQ ID NO: 15 - DNA, 31 bp, synthetic construct
  source 1..31: mol_type = other DNA
ttgacgttagatgtaacctgagggttataat

SEQ ID NO: 16 - DNA, 31 bp, synthetic construct
  source 1..31: mol_type = other DNA
ttgacccttacttgaacgttactgttataat

SEQ ID NO: 17 - DNA, 31 bp, synthetic construct
  source 1..31: mol_type = other DNA
ttgactctagctttagggtattgactataat

SEQ ID NO: 18 - DNA, 31 bp, synthetic construct
  source 1..31: mol_type = other DNA
ttgactctaaggttatacttggcgctataat

SEQ ID NO: 19 - DNA, 42 bp, synthetic construct
  source 1..42: mol_type = other DNA
aagcgctcaaatgaaggtttgagtgaaggtaagggttataat

SEQ ID NO: 20 - DNA, 42 bp, synthetic construct
  source 1..42: mol_type = other DNA
aagcgcatcttggctaaccctgaagctacttcactgtataat

SEQ ID NO: 21 - DNA, 42 bp, synthetic construct
  source 1..42: mol_type = other DNA
aagcgcttaactatgacgttaaattgaactatagggtataat

SEQ ID NO: 22 - DNA, 42 bp, synthetic construct
  source 1..42: mol_type = other DNA
aagcgcatgtagttgaccttaacgtgagggttatagtataat

SEQ ID NO: 23 - DNA, 42 bp, synthetic construct
  source 1..42: mol_type = other DNA
aagcgcgaaccttgaggttgaattgaaggttagggttataat

SEQ ID NO: 24 - DNA, 29 bp, synthetic construct
  source 1..29: mol_type = other DNA
ttgacattgatatgtgtacttattataat

SEQ ID NO: 25 - DNA, 29 bp, synthetic construct
  source 1..29: mol_type = other DNA
ttgacaatagtcagtgcactgagtataat

SEQ ID NO: 26 - DNA, 29 bp, synthetic construct
  source 1..29: mol_type = other DNA
ttgacattgatatgtgtacttattataat

SEQ ID NO: 27 - DNA, 29 bp, synthetic construct
  source 1..29: mol_type = other DNA
ttgacacatatgatatgtatacgtataat

SEQ ID NO: 28 - DNA, 29 bp, synthetic construct
  source 1..29: mol_type = other DNA
ttgacattcagttcactgaatcttataat

SEQ ID NO: 29 - DNA, 29 bp, synthetic construct
  source 1..29: mol_type = other DNA
ttgacaatcgagcgtgtacgctctataat

SEQ ID NO: 30 - DNA, 29 bp, synthetic construct
  source 1..29: mol_type = other DNA
ttgacattcttcagtgtactgaatataat

SEQ ID NO: 31 - DNA, 29 bp, synthetic construct
  source 1..29: mol_type = other DNA
ttgacattgcttcgtatacgaactataat

SEQ ID NO: 32 - DNA, 29 bp, synthetic construct
  source 1..29: mol_type = other DNA
ttgacagctggatcattggtgtgtataat